SEQUENCE LISTING (48 sequences)

SEQ ID NO: 1 - protein, 625 aa, Homo sapiens [sequence omitted]

SEQ ID NO: 2 - DNA, 3278 bp, Homo sapiens
  source 1..3278: mol_type = genomic DNA
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 3 - protein, 5 aa, synthetic construct
TAAMS

SEQ ID NO: 4 - protein, 17 aa, synthetic construct
GISGSGSSTYYADSVKG

SEQ ID NO: 5 - protein, 13 aa, synthetic construct
ELSYLYSGYYFDY

SEQ ID NO: 6 - protein, 7 aa, synthetic construct
GFTFSTA

SEQ ID NO: 7 - protein, 6 aa, synthetic construct
SGSGSS

SEQ ID NO: 8 - protein, 13 aa, synthetic construct
ELSYLYSGYYFDY

SEQ ID NO: 9 - protein, 122 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSS

SEQ ID NO: 10 - DNA, 366 bp, synthetic construct
  source 1..366: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 11 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1356 bp, synthetic construct
  source 1..1356: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 13 aa, synthetic construct
SGSSSNIGSNDVS

SEQ ID NO: 14 - protein, 7 aa, synthetic construct
KNYNRPS

SEQ ID NO: 15 - protein, 11 aa, synthetic construct
SAWDQRQFDVV

SEQ ID NO: 16 - protein, 9 aa, synthetic construct
SSSNIGSND

SEQ ID NO: 17 - skipped

SEQ ID NO: 18 - protein, 8 aa, synthetic construct
WDQRQFDV

SEQ ID NO: 19 - protein, 110 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 20 - DNA, 330 bp, synthetic construct
  source 1..330: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtccta

SEQ ID NO: 21 - protein, 216 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - DNA, 648 bp, synthetic construct
  source 1..648: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 23 - protein, 5 aa, synthetic construct
TAAMS

SEQ ID NO: 24 - protein, 17 aa, synthetic construct
GISGSGSSTYYADSVKG

SEQ ID NO: 25 - protein, 13 aa, synthetic construct
ELSYLYSGYYFDY

SEQ ID NO: 26 - protein, 7 aa, synthetic construct
GFTFSTA

SEQ ID NO: 27 - protein, 6 aa, synthetic construct
SGSGSS

SEQ ID NO: 28 - protein, 13 aa, synthetic construct
ELSYLYSGYYFDY

SEQ ID NO: 29 - protein, 122 aa, synthetic construct; identical to SEQ ID NO: 9

SEQ ID NO: 30 - DNA, 366 bp, synthetic construct
  source 1..366: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagc

SEQ ID NO: 31 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1356 bp, synthetic construct
  source 1..1356: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 33 - protein, 13 aa, synthetic construct
SGSSSNIGSNDVS

SEQ ID NO: 34 - protein, 7 aa, synthetic construct
KNYNRPS

SEQ ID NO: 35 - protein, 11 aa, synthetic construct
SAWDQRQFDVV

SEQ ID NO: 36 - protein, 9 aa, synthetic construct
SSSNIGSND

SEQ ID NO: 37 - skipped

SEQ ID NO: 38 - protein, 8 aa, synthetic construct
WDQRQFDV

SEQ ID NO: 39 - protein, 110 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVL

SEQ ID NO: 40 - DNA, 330 bp, synthetic construct
  source 1..330: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctg

SEQ ID NO: 41 - protein, 216 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 648 bp, synthetic construct
  source 1..648: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 43 - protein, 8 aa, synthetic construct
GFTFSTAA

SEQ ID NO: 44 - protein, 8 aa, synthetic construct
ISGSGSST

SEQ ID NO: 45 - protein, 15 aa, synthetic construct
ARELSYLYSGYYFDY

SEQ ID NO: 46 - protein, 10 aa, synthetic construct
GFTFSTAAMS

SEQ ID NO: 47 - protein, 8 aa, synthetic construct
SSNIGSND

SEQ ID NO: 48 - protein, 6 aa, synthetic construct
HHHHHH

(19) United States
(12) Patent Application Publication
(10) Pub. No.: US 2024/0294669 A1
(43) Pub. Date: Sep. 5, 2024

(54) DOSING REGIMENS OF FACTOR XI/XIA ANTIBODIES

(71) Applicant: Anthos Therapeutics, Inc., Cambridge, MA (US)

(72) Inventors: Debra A. FREEDHOLM, Cambridge, MA (US); Daniel M. BLOOMFIELD, Belmar, NJ (US); Royston J. GLASSPOOL, Cambridge, MA (US); Jonathan E. FREEMAN, Cambridge, MA (US); Yasser KHDER, Rosenau (FR)

(21) Appl. No.: 18/666,945

(22) Filed: May 17, 2024

Related U.S. Application Data
(63) Continuation of application No. PCT/US2022/080131, filed on Nov. 18, 2022.
(60) Provisional application No. 63/281,024, filed on Nov. 18, 2021.
(60) Provisional application No. 63/287,633, filed on Dec. 9, 2021.
(60) Provisional application No. 63/355,314, filed on Jun. 24, 2022.

(51) Int. Cl. C07K 16/36; A61K 39/00; A61K 47/22; A61K 47/26; A61P 7/02

(52) U.S. Cl. CPC C07K 16/36; A61K 47/22; A61K 47/26; A61P 7/02; A61K 2039/505; A61K 2039/54; A61K 2039/545; C07K 2317/76

(57) ABSTRACT

This disclosure relates to dosage regimens for anti-Factor XI and/or activated Factor XI (Factor XIa) antibodies or antigen-binding fragments thereof, pharmaceutical formulations comprising the same, and pharmaceutical formulations for use in the treatment of thromboembolic disorders or related conditions. Also provided are pharmaceutical formulations of anti-Factor XI and/or activated Factor XI (Factor XIa) antibodies, or antigen-binding fragments thereof. Also provided are methods of treating patients having thrombocytopenias with anti-Factor XI and/or activated Factor XI (Factor XIa) antibodies, or antigen-binding fragments thereof.

### CROSS-REFERENCE TO RELATED APPLICATIONS

This application is a continuation of International (PCT) Patent Application No. PCT/US2022/080131, filed Nov. 18, 2022, which claims the benefit of and priority to U.S. Provisional Patent Application No. 63/281,024, filed Nov. 18, 2021; U.S. Provisional Patent Application No. 63/287,633, filed Dec. 9, 2021; and U.S. Provisional Patent Application No. 63/355,314, filed Jun. 24, 2022, the disclosures of each of which are incorporated herein by reference in their entireties for all purposes.

### SEQUENCE LISTING

This application contains a Sequence Listing XML, which has been submitted electronically and is hereby incorporated by reference in its entirety. Said XML copy, created on May 16, 2024, is named ATD-011WOC1_SL.XML and is 52,209 bytes in size.

### FIELD OF THE DISCLOSURE

The present disclosure relates to dosage regimens for anti-Factor XI and/or activated Factor XI (Factor XIa) antibodies or antigen-binding fragments thereof, pharmaceutical formulations comprising the same, and pharmaceutical formulations for use in the treatment of thromboembolic disorders or related conditions. Also provided are pharmaceutical formulations of anti-Factor XI and/or activated Factor XI (Factor XIa) antibodies, or antigen-binding fragments thereof.

### BACKGROUND

There exists a high unmet medical need for safer therapies to reduce thromboembolic complications such as stroke, systemic embolism, cognitive decline and mortality, with comparable or improved efficacy to that exhibited by existing therapies, and with a lower risk of bleeding.

Factor XI (FXI) is a serine protease functioning both in the intrinsic and extrinsic coagulation pathways. Factor XI exists in the zymogen form as a homodimer; upon cleavage of the peptide bond at R369-1370, Factor XI is activated (Factor XIa, FXIa). FXI plays a minor role in normal hemostasis in a high tissue factor environment but does play a key role in thrombosis. Genetic Factor XI deficiency is associated with decreased incidence of ischemic stroke and venous thromboembolic events (Salomon et al. (2008); Salomon, et al. (2011) Thromb Haemost.; 105:269-73). Bleeding manifestations in subjects with Factor XI deficiency are infrequent, often mild, result from injury or trauma, and very rarely affect critical organs (Salomon et al. (2011)).

Antibodies that bind Factor XI and/or Factor XIa have been studied. For example, WO 2016/207858 describes one such anti-Factor XI and/or Factor XIa antibody, disclosed herein in Table 1 as Antibody 1. The present disclosure adds to these developments and provides further clinical methods, including dosage regimens, to treat patients with specific thromboembolic disorders with desired safety and efficacy. Furthermore, the present disclosure adds to the earlier developments in the field by providing pharmaceutical formulations comprising such FXI and/or FXIa antibodies that are sufficiently stable and suitable for administration to patients.

### SUMMARY

The present disclosure provides dosage regimens for anti-Factor XI and/or Factor XIa antibodies or antigen-binding fragments thereof, or pharmaceutical formulations comprising the same.

Accordingly, in one aspect, provided herein is a method of treating a disease or disorder in a subject in need thereof, the method comprising intravenously administering to the subject a first dose of about 150 mg of an isolated anti-Factor XI (FXI) and/or anti-activated Factor XI (FXIa) antibody, or an antigen-binding fragment thereof, and subcutaneously administering to the subject a second dose of the isolated anti-FXI and/or anti-FXIa antibody, or the antigen-binding fragment thereof.

In some embodiments, the second dose comprises about 150 mg of the isolated anti-FXI and/or anti-FXIa antibody, or the antigen-binding fragment thereof. In some embodiments, the first dose of the isolated anti-FXI and/or anti-FXIa antibody, or the antigen-binding fragment thereof, is formulated as an intravenous drug delivery formulation comprising about 150 mg of the antibody, or the antigen-binding fragment thereof. In some embodiments, the second dose of the isolated anti-FXI and/or anti-FXIa antibody, or the antigen-binding fragment thereof, is formulated as a subcutaneous drug delivery formulation comprising about 150 mg of the antibody or the antigen-binding fragment thereof.

In some embodiments, the antibody is a human monoclonal antibody. In some embodiments, the antibody is a human IgG1 isotype. In some embodiments, the antibody comprises D265A and P329A substitutions in the Fc domain.

In some embodiments, the antibody or antigen-binding fragment thereof is administered in a drug delivery formulation comprising a histidine buffer at a concentration of about 20 mM. In some embodiments, the antibody or antigen-binding fragment thereof is administered in a drug delivery formulation comprising sucrose at a concentration of about 220 mM. In some embodiments, the antibody or antigen-binding fragment thereof is administered in a drug delivery formulation comprising polysorbate 20 at a concentration of about 0.04%. In some embodiments, the antibody or antigen-binding fragment thereof is administered in a drug delivery formulation at pH 5.5. In some embodiments, the antibody or antigen-binding fragment thereof is administered in an intravenous drug delivery formulation, the intravenous drug delivery formulation further comprises about 5% glucose.

In some embodiments, the subject has a cancer. In certain embodiments, the subject has an active cancer. In certain embodiments, the subject has a cancer selected from the group consisting of gastrointestinal cancer and genitourinary cancer. In some embodiments, the subject is at high risk of venous thromboembolism. In some embodiments, the subject has had one or more previous venous thromboembolisms.

In some embodiments, the method further comprises one or more additional subcutaneous doses of the antibody or antigen-binding fragment thereof. In some embodiments, the method comprises administering five subcutaneous doses of the antibody or antigen-binding fragment thereof. In some embodiments, the antibody or antigen-binding fragment thereof is administered subcutaneously about once a month. In some embodiments, the antibody or antigen-binding fragment thereof is administered intravenously on day 1 and is administered subcutaneously on days 31, 61, 91, 121, and 151. In some embodiments, the subject is treated for about six months.

In another aspect, provided herein is a method of treating a subject with a cancer, wherein the method comprises administering a drug delivery formulation comprising about 150 mg of an isolated anti-Factor XI (FXI) and/or anti-activated Factor XI (FXIa) antibody or antigen-binding fragment thereof to the subject in need thereof, wherein the drug delivery formulation is administered once intravenously and subsequently is administered subcutaneously about once a month, and wherein the subject is treated for about six months.

In some embodiments, the cancer is selected from the group consisting of gastrointestinal cancer and genitourinary cancer.

In another aspect, provided herein is a method of treating a primate subject at risk of thrombosis, wherein the method comprises administering to the primate subject a single dose of a drug delivery formulation comprising:
(a) a therapeutically effective amount of an isolated anti-Factor XI (FXI) and/or anti-activated Factor XI (FXIa) antibody, or antigen-binding fragment thereof at a concentration of about 150 mg;
(b) a histidine buffer at a concentration of about 20 mM;
(c) sucrose at a concentration of about 220 mM; and
(d) polysorbate-20 at a concentration of about 0.04% (v/v),
(e) a diluent comprising glucose,
at pH 5.5,
and wherein the administering is before or during formation of a blood clot.

In some embodiments, the primate subject is a baboon. In some embodiments, the primate subject is a human. In some embodiments, the thrombosis is an experimentally-induced thrombosis. In some embodiments, the primate subject is at risk of vascular graft thrombosis. In some embodiments, the single dose is administered to prevent thrombosis. In some embodiments, the single dose is administered to treat thrombosis. In some embodiments, the single dose is parenteral or intravenous. In certain embodiments, the single dose is followed by subsequent doses. In certain embodiments, the subsequent doses are parenteral.

In some embodiments, about 1 mg/kg is the therapeutically effective amount of the anti-Factor XI (FXI) and/or anti-activated Factor XI (FXIa) antibody or antigen-binding fragment thereof, for administration to the primate subject. In some embodiments, about 150 mg is the therapeutically effective amount of the anti-Factor XI (FXI) and/or anti-activated Factor XI (FXIa) antibody or antigen-binding fragment thereof, for administration to the primate subject.

In another aspect, provided herein is a method of treating a subject having a thrombocytopenia, wherein the thrombocytopenia is selected from the group consisting of: chemotherapy-induced thrombocytopenia, congenital thrombocytopenia, thrombocytopenia associated with infection, and idiopathic thrombocytopenia, the method comprising administering a therapeutically effective amount of a Factor XI and/or Factor XIa antibody, or an antigen-binding fragment thereof, to the subject in need thereof.

In some embodiments, the subject having a thrombocytopenia has a cancer.

In some embodiments, the subject having a thrombocytopenia has cirrhosis.

In some embodiments, the subject having a thrombocytopenia has idiopathic thrombocytopenia purpura (ITP).

In another aspect, provided herein is a method of treating a cancer subject having a chemotherapy-induced thrombocytopenia, wherein the method comprises administering a therapeutically effective amount of a Factor XI and/or Factor XIa antibody, or an antigen-binding fragment thereof, to the cancer subject in need thereof.

In some embodiments, the subject or the cancer subject is afflicted with or at risk of developing a thromboembolic disorder.

In some embodiments of the above aspects, the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

In some embodiments of the above aspects, the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

In some embodiments, the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

In some embodiments, the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39. In some embodiments, the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto. In some embodiments, the antibody comprises a heavy chain comprising an amino acid sequence of SEQ ID NO: 31 and a light chain comprising an amino acid sequence of SEQ ID NO: 41.

In some embodiments, the antibody is a human monoclonal antibody. In some embodiments, the antibody is a human IgG1 isotype. In certain embodiments, the antibody comprises D265A and P329A substitutions in the Fc domain.

In some embodiments, the administering of the antibody or antigen-binding fragment thereof does not affect platelet aggregation in the subject as compared to platelet aggregation prior to the administering. In certain embodiments, the platelet aggregation is measured by impedance platelet aggregometry. In certain embodiments, the platelet aggregation is induced by collagen, adenosine 5′-diphosphate (ADP), or thrombin receptor activating peptide-6 (TRAP-6). In certain embodiments, the platelet aggregation is determined ex vivo or in vitro.

In some embodiments, the antibody or antigen-binding fragment thereof is administered intravenously.

In some embodiments, the antibody or antigen-binding fragment thereof is administered subcutaneously.

In some embodiments, a first dose of the antibody or antigen-binding fragment thereof is administered intravenously and a second dose of the antibody or antigen-binding fragment is administered subcutaneously. In certain embodiments, the method further comprises one or more additional doses of the antibody or antigen-binding fragment thereof administered subcutaneously following the administering of the second dose.

In some embodiments, the antibody or antigen-binding fragment thereof is administered once a month.

Other embodiments and details of the disclosure are presented herein below.

### BRIEF DESCRIPTION OF THE DRAWINGS

FIG. 1 depicts line graphs showing a time course of activated partial thromboplastin time (aPTT) following 1 mg/kg intravenous administration of Antibody 1 in four baboons.

FIGS. 2A-2B depict bleeding time (FIG. 2A) and bleeding volume (FIG. 2B) before and during each arteriovenous (AV) shunt experiment.

FIGS. 3A-3B depict platelet deposition in the tail segment of the AV shunt for collagen-coating (FIG. 3A) or collagen+tissue factor (TF) coating (FIG. 3B). Vertical dotted line depicts time of Antibody 1 administration and the arrow shows the inflection point where platelet deposition was halted. Time between the dotted vertical line and arrow measures time between drug administration and the point where platelet deposition rate turns negative.

FIGS. 4A-4F depict platelet deposition in the tail segment of the AV shunt for the three treated baboons. FIG. 4A, FIG. 4B, and FIG. 4C showing collagen-coating and FIG. 4D, FIG. 4E, and FIG. 4F show collagen+TF coating.

FIGS. 5A-5B show platelet deposition in the collagen-coated (FIG. 5A) or collagen+TF coated (FIG. 5B) segments of the AV shunt. Data are an average of the three baboons.

FIGS. 6A-6D depict fibrin content of the thrombi in the collagen-coated graft (FIG. 6A), collagen+TF-coated graft (FIG. 6B), tail segment downstream to a collagen-coated graft (FIG. 6C), and tail segment downstream to a collagen+TF-coated graft (FIG. 6D).

FIGS. 7A-7B depict in vitro platelet aggregation in donor whole blood supplemented with vehicle, Antibody 1, or abciximab following induction with collagen (FIG. 7A) or TRAP-6 (FIG. 7B).

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “FXI protein,” “FXI antigen,” and “FXI” are used interchangeably, and refers to the Factor XI protein in different species. Factor XI is the mammalian plasma coagulation Factor XI, a glycoprotein present in human plasma at a concentration of 25-30 nM as a zymogen that when converted by limited proteolysis to an active serine protease, participates in the intrinsic pathway of blood coagulation.

The terms “FXIa protein,” “FXIa antigen,” and “FXIa”, are used interchangeably, and refers to the activated FXI protein in different species. The zymogen Factor XI is converted into its active form, the coagulation Factor Xla (FXIa), either via the contact phase of blood coagulation or through thrombin-mediated activation on the platelet surface. During this activation of factor XI, an internal peptide bond is cleaved in each of the two chains, resulting in the activated factor Xla, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor II/Thrombin activation. For example, human FXI has the sequence as set out in Table 1 (SEQ ID NO:1) and has been described in previous reports and literature (Mandle R J Jr, et al. (1979) Blood; 54(4):850; NCBI Reference Sequence: AAA51985).

In the context of this present disclosure, the terms “FXI” and “FXIa” (and the like) include mutants and variants of the natural FXI and FXIa protein, respectively, which have substantially the same amino acid sequence as that of the native primary structure (amino acid sequence) described in the above-mentioned reports.

The term “catalytic domain,” “serine protease catalytic domain,” and similar terms as used herein, means amino acids Ile370 to Val607, as counted from the Glu1 at the N-terminus of the mature protein that is in circulation. It can also be described as residues 388-625 at the C-terminus of FXI. As used herein, the term “active site” means the catalytic triad comprised of the amino acids His413, Asp462 and Ser557. (Bane and Gailani (2014) Drug Disc. 19(9)).

The term “antibody” as used herein means a whole antibody and any antigen binding fragment (e.g., “antigen-binding portion”) or single chain thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some specific aspects, an antibody can be a monoclonal antibody, human antibody, humanized antibody, camelid antibody, or chimeric antibody. Antibodies can be of any isotype (e.g., IgG, IgE, IgM, IgD, IgA and IgY), class (e.g., IgG1, IgG2, IgG3, IgG4, IgA1 and IgA2) or subclass.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “drug delivery formulation” or “intravenous drug delivery formulation” refers to a pharmaceutical formulation comprising the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans. In certain embodiments, the subject is a human. As used herein “primate subject” is inclusive of both human and non-human primates. In certain embodiments, the subject is a baboon model of thrombosis, described, for example, in Gruber et al. Blood, 1989 Feb. 15; 73(3):639-42 and Crosby et al. Arterioscler Thromb Vasc Biol, 2013 July; 33(7):1670-8.

A “thromboembolic disorder,” or similar terms as used herein, refer to any number of conditions or diseases in which the intrinsic and/or common coagulation pathways are aberrantly activated or are not naturally deactivated (e.g., without therapeutic means). These conditions include but are not limited to thromboembolic stroke and other types of stroke of ischemic origin, atrial fibrillation, stroke prevention in atrial fibrillation (SPAF), deep vein thrombosis, venous thromboembolism, and pulmonary embolism. These can also include prevention and treatment of catheter-related thrombosis (e.g., Hickman catheter in oncology patients) in which catheters become thrombosed, and extracorporeal membrane oxygenation (ECMO), in which the tubing and oxygenation membrane develops clots.

A “thromboembolic disorder” or similar terms as used herein, can also refer to any number of the following, which the anti-FXI and/or FXIa antibodies or antigen binding fragments thereof of the present disclosure can be used to prevent or treat:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
venous thromboembolism, wherein the subject is a pediatric subject (pediatric VTE);
cerebral and cardiovascular events in secondary prevention after transient ischemic attack (TIA) or non-disabling stroke and prevention of thromboembolic events in heart failure with sinus rhythm;
hemorrhagic stroke;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins or arteries like catheter, pacemaker wires, synthetic arterial grafts; mechanical or biological heart valves or left ventricular assist device;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
non-central nervous systemic embolism (non-CNS systemic embolism);
thrombosis in patients with invasive and non-invasive cancer malignancies (e.g., CAT);
thrombosis over an indwelling catheter;
thrombosis and thromboembolism in severely ill patients;
cardiac thrombosis and thromboembolism, including but not limited to cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
thromboembolism in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease; and
thrombosis and thromboembolism in patients undergoing hemodialysis and in patients undergoing extra-corporal membrane oxygenation.

As used herein, the term “trough” or “trough level” refers to the lowest concentration reached by a drug before the next dose of the drug is administered. In certain embodiments, inhibition of Factor XI/Factor XIa at trough is greater than about 50% (e.g., greater than about 60%, greater than about 70%, greater than about 80%, or greater than about 90%). In certain embodiments, inhibition of Factor XI/Factor XIa at trough is greater than about 80%. In certain embodiments, inhibition of Factor XI/Factor XIa at trough is greater than about 90%.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By “therapeutic benefit,” what is meant is eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

In certain embodiments of the methods described herein, the subject is treatment naïve, i.e., has never received any form of anticoagulant therapy prior to treatment with an anti-Factor XI/XIa antibody described herein, e.g., Antibody 1. In certain embodiments of the methods described herein, the subject has received a stable treatment of a recommended dose of a new oral anticoagulant (NOAC), e.g., prior to treatment with an anti-Factor XI/XIa antibody described herein, e.g., Antibody 1. In certain embodiments, the subject has received a direct oral anticoagulant (DOAC) e.g., prior to treatment with an anti-Factor XI/XIa antibody described herein, e.g., Antibody 1. In certain embodiments, the subject has received a Vitamin K antagonist (VKA) e.g., prior to treatment with an anti-Factor XI/XIa antibody described herein, e.g., Antibody 1.

As used herein, the term “vial” refers to a container that holds the drug product. In some embodiments, the vial may be a vial, a bag, a pen, or a syringe. In some embodiments, the vial may be a vial, e.g., a glass vial.

As used herein, the term “drug product” refers to an anti-Factor XI/XIa antibody described herein, e.g., Antibody 1 as disclosed in Table 1, and excipients, e.g., a histidine buffer, a sugar, and a polysorbate.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Anti-Factor XI and/or Activated Factor XI (Factor XIa) Antibodies

In some embodiments, the present disclosure provides pharmaceutical formulations comprising antibodies that bind FXI and/or FXIa protein (e.g., human, rabbit, cynomolgus monkey, and baboon FXI and/or FXIa), wherein the antibodies comprise a heavy chain variable domain (VH) having an amino acid sequence of SEQ ID NOs: 9 or 29, the formulations comprise a histidine buffer; a sugar or sugar alcohol; and a polysorbate, and the pH of the formulation is between pH 5.0 to 6.0. In certain embodiments, the antibodies comprise a VH having an amino acid sequence of SEQ ID NO:29.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides a pharmaceutical formulations of antibodies that specifically bind to a FXI and/or FXIa protein, wherein the antibodies comprise a VH CDR having an amino acid sequence of any one of the VH CDRs listed in Table 1, infra, the formulations comprise a histidine buffer; a sugar or sugar alcohol; and a polysorbate; and the pH of the formulation is between pH 5.0 to 6.0. In particular, the present disclosure provides pharmaceutical formulations of antibodies that specifically bind to a FXI and/or FXIa protein (e.g., human, rabbit, cynomolgus monkey, and baboon FXI and/or FXIa), wherein the antibodies comprise (or alternatively, consist of) one, two, three, or more VH CDRs having an amino acid sequence of any of the VH CDRs listed in Table 1, infra, the formulations comprise a histidine buffer; a sugar or sugar alcohol; and a polysorbate; and the pH of the formulation is between pH 5.0 to 6.0. (see PCT International Patent Application No. PCT/IB2016/053790 filed on Jun. 24, 2016, and published as WO2016/207858, which is hereby incorporated by reference in its entirety).

In some embodiments, the present disclosure provides pharmaceutical formulations of antibodies that specifically bind to a FXI/FXIa protein, said antibodies comprising a light chain variable domain (VL) having an amino acid sequence of SEQ ID NOs: 19 or 39, for use in the methods described herein (e.g., methods for treating a subject afflicted with or at risk of developing a thromboembolic disorder), the formulations comprise a histidine buffer; a sugar or sugar alcohol; and a polysorbate; and the pH of the formulation is between pH 5.0 to 6.0. In certain embodiments, the antibodies comprise a VL having an amino acid sequence of SEQ ID NO:39.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides pharmaceutical formulations of antibodies that specifically bind to a FXI and/or FXIa protein (e.g., human, rabbit, cynomolgus monkey, and baboon FXI and/or FXIa), for use in the methods described herein (e.g., methods for treating a subject afflicted with or at risk of developing a thromboembolic disorder), the antibodies comprising a VL CDR having an amino acid sequence of any one of the VL CDRs listed in Table 1, infra; the formulations comprise a histidine buffer; a sugar or sugar alcohol; and a polysorbate; and the pH of the formulation is between pH 5.0 to 6.0. The antibodies that specifically bind to an FXIa protein (e.g., human, rabbit, cynomolgus monkey, and baboon FXI and/or FXIa), may comprise (or alternatively, consist of) one, two, three or more VL CDRs having an amino acid sequence of any of the VL CDRs listed in Table 1, infra.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In some embodiments, other antibodies for use in the methods described herein (e.g., methods for treating a subject afflicted with or at risk of developing a thromboembolic disorder) include amino acids that have been mutated, yet have at least 60, 70, 80, 85, 90 or 95 percent identity in the CDR regions with the CDR regions depicted in the sequences described in Table 1. In some embodiments, the antibodies include mutant amino acid sequences wherein no more than 1, 2, 3, 4 or 5 amino acids have been mutated in the CDR regions when compared with the CDR regions depicted in the sequence described in Table 1.

TABLE 1
Examples of FXI/FXIa Antibodies, Fabs and FXI/FXIa Proteins

| | Sequence | |
|---|---|---|
| | Identifier | |
| | (SEQ ID | |
| Sequence Description | NO:) | Amino acid or polynucleotide sequence |
| Human FXIa full- | 1 | MIFLYQVVHFILFTSVSGECVTQLLKDTCFEGGDIT |
| length protein | | TVFTPSAKYCQVVCTYHPRCLLFTFTAESPSEDPTR |
| sequence (NCBI | | WFTCVLKDSVTETLPRVNRTAAISGYSFKQCSHQI |
| Reference Sequence: | | SACNKDIYVDLDMKGINYNSSVAKSAQECQERCT |
| AAA51985) | | DDVHCHFFTYATRQFPSLEHRNICLLKHTQTGTPT |
| | | RITKLDKVVSGFSLKSCALSNLACIRDIFPNTVFAD |
| | | SNIDSVMAPDAFVSGRICTHHPGCLFFTFFSQEWP |
| | | KESQRNLCLLKTSESGLPSTRIKKSKALSGFSLQSC |
| | | RHSIPVFCHSSFYHDTDFLGEELDIVAAKSHEACQ |
| | | KLCTNAVRCQFFTYTPAQASCNEGKGKCYLKLSS |
| | | NGSPTKILHGRGGISGYTLRLCKMDNECTTKIKPRI |
| | | VGGTASVRGEWPWQVTLHTTSPTQRHLCGGSIIG |
| | | NQWILTAAHCFYGVESPKILRVYSGILNQSEIKEDT |
| | | SFFGVQEIIIHDQYKMAESGYDIALLKLETTVNYTD |
| | | SQRPICLPSKGDRNVIYTDCWVTGWGYRKLRDKI |
| | | QNTLQKAKIPLVTNEECQKRYRGHKITHKMICAG |
| | | YREGGKDACKGDSGGPLSCKHNEVWHLVGITSW |
| | | GEGCAQRERPGVYTNVVEYVDWILEKTQAV |
| Human FXIa full- | 2 | AGGCACACAGGCAAAATCAAGTTCTACATCTGT |
| length nucleotide | | CCCTGTGTATGTCACTTGTTTGAATACGAAATAA |
| sequence (NCBI | | AATTAAAAAAATAAATTCAGTGTATTGAGAAAG |
| Reference Sequence: | | CAAGCAATTCTCTCAAGGTATATTTCTGACATAC |
| NM 000128.3) | | TAAGATTTTAACGACTTTCACAAATATGCTGTAC |
| | | TGAGAGAGAATGTTACATAACATTGAGAACTAG |
| | | TACAAGTAAATATTAAAGTGAAGTGACCATTTC |
| | | CTACACAAGCTCATTCAGAGGAGGATGAAGACC |
| | | ATTTTGGAGGAAGAAAAGCACCCTTATTAAGAA |
| | | TTGCAGCAAGTAAGCCAACAAGGTCTTTTCAGG |
| | | ATGATTTTCTTATATCAAGTGGTACATTTCATTT |
| | | TATTTACTTCAGTTTCTGGTGAATGTGTGACTCA |
| | | GTTGTTGAAGGACACCTGCTTTGAAGGAGGGGA |
| | | CATTACTACGGTCTTCACACCAAGCGCCAAGTA |
| | | CTGCCAGGTAGTCTGCACTTACCACCCAAGATG |
| | | TTTACTCTTCACTTTCACGGCGGAATCACCATCT |
| | | GAGGATCCCACCCGATGGTTTACTTGTGTCCTGA |
| | | AAGACAGTGTTACAGAAACACTGCCAAGAGTGA |
| | | ATAGGACAGCAGCGATTTCTGGGTATTCTTTCAA |
| | | GCAATGCTCACACCAAATAAGCGCTTGCAACAA |
| | | AGACATTTATGTGGACCTAGACATGAAGGGCAT |
| | | AAACTATAACAGCTCAGTTGCCAAGAGTGCTCA |
| | | AGAATGCCAAGAAAGATGCACGGATGACGTCCA |
| | | CTGCCACTTTTTCACGTACGCCACAAGGCAGTTT |
| | | CCCAGCCTGGAGCATCGTAACATTTGTCTACTGA |
| | | AGCACACCCAAACAGGGACACCAACCAGAATA |
| | | ACGAAGCTCGATAAAGTGGTGTCTGGATTTTCA |
| | | CTGAAATCCTGTGCACTTTCTAATCTGGCTTGTA |
| | | TTAGGGACATTTTCCCTAATACGGTGTTTGCAGA |
| | | CAGCAACATCGACAGTGTCATGGCTCCCGATGC |
| | | TTTTGTCTGTGGCCGAATCTGCACTCATCATCCC |
| | | GGTTGCTTGTTTTTTACCTTCTTTTCCCAGGAATG |
| | | GCCCAAAGAATCTCAAAGAAATCTTTGTCTCCTT |
| | | AAAACATCTGAGAGTGGATTGCCCAGTACACGC |
| | | ATTAAAAAGAGCAAAGCTCTTTCTGGTTTCAGTC |
| | | TACAAAGCTGCAGGCACAGCATCCCAGTGTTCT |
| | | GCCATTCTTCATTTTACCATGACACTGATTTCTT |
| | | GGGAGAAGAACTGGATATTGTTGCTGCAAAAAG |
| | | TCACGAGGCCTGCCAGAAACTGTGCACCAATGC |
| | | CGTCCGCTGCCAGTTTTTTACCTATACCCCAGCC |
| | | CAAGCATCCTGCAACGAAGGGAAGGGCAAGTGT |
| | | TACTTAAAGCTTTCTTCAAACGGATCTCCAACTA |
| | | AAATACTTCACGGGAGAGGAGGCATCTCTGGAT |
| | | ACACATTAAGGTTGTGTAAAATGGATAATGAGT |
| | | GTACCACCAAAATCAAGCCCAGGATCGTTGGAG |
| | | GAACTGCGTCTGTTCGTGGTGAGTGGCCGTGGC |
| | | AGGTGACCCTGCACACAACCTCACCCACTCAGA |
| | | GACACCTGTGTGGAGGCTCCATCATTGGAAACC |
| | | AGTGGATATTAACAGCCGCTCACTGTTTCTATGG |
| | | GGTAGAGTCACCTAAGATTTTGCGTGTCTACAGT |
| | | GGCATTTTAAATCAATCTGAAATAAAAGAGGAC |
| | | ACATCTTTCTTTGGGGTTCAAGAAATAATAATCC |
| | | ATGATCAGTATAAAATGGCAGAAAGCGGGTATG |
| | | ATATTGCCTTGTTGAAACTGGAAACCACAGTGA |
| | | ATTACACAGATTCTCAACGACCCATATGCCTGCC |
| | | TTCCAAAGGAGATAGAAATGTAATATACACTGA |
| | | TTGCTGGGTGACTGGATGGGGGTACAGAAAACT |
| | | AAGAGACAAAATACAAAATACTCTCCAGAAAGC |
| | | CAAGATACCCTTAGTGACCAACGAAGAGTGCCA |
| | | GAAGAGATACAGAGGACATAAAATAACCCATA |
| | | AGATGATCTGTGCCGGCTACAGGGAAGGAGGGA |
| | | AGGACGCTTGCAAGGGAGATTCGGGAGGCCCTC |
| | | TGTCCTGCAAACACAATGAGGTCTGGCATCTGG |
| | | TAGGCATCACGAGCTGGGGCGAAGGCTGTGCTC |
| | | AAAGGGAGCGGCCAGGTGTTTACACCAACGTGG |
| | | TCGAGTACGTGGACTGGATTCTGGAGAAAACTC |
| | | AAGCAGTGTGAATGGGTTCCCAGGGGCCATTGG |
| | | AGTCCCTGAAGGACCCAGGATTTGCTGGGAGAG |
| | | GGTGTTGAGTTCACTGTGCCAGCATGCTTCCTCC |
| | | ACAGTAACACGCTGAAGGGGCTTGGTGTTTGTA |
| | | AGAAAATGCTAGAAGAAAACAAACTGTCACAA |
| | | GTTGTTATGTCCAAAACTCCCGTTCTATGATCGT |
| | | TGTAGTTTGTTTGAGCATTCAGTCTCTTTGTTTTT |
| | | GATCACGCTTCTATGGAGTCCAAGAATTACCAT |
| | | AAGGCAATATTTCTGAAGATTACTATATAGGCA |
| | | GATATAGCAGAAAATAACCAAGTAGTGGCAGTG |
| | | GGGATCAGGCAGAAGAACTGGTAAAAGAAGCC |
| | | ACCATAAATAGATTTGTTCGATGAAAGATGAAA |
| | | ACTGGAAGAAAGGAGAACAAAGACAGTCTTCA |
| | | CCATTTTGCAGGAATCTACACTCTGCCTATGTGA |
| | | ACACATTTCTTTTGTAAAGAAAGAAATTGATTGC |
| | | ATTTAATGGCAGATTTTCAGAATAGTCAGGAAT |
| | | TCTTGTCATTTCCATTTTAAAATATATATTAAAA |
| | | AAAATCAGTTCGAGTAGACACGAGCTAAGAGTG |
| | | AATGTGAAGATAACAGAATTTCTGTGTGGAAGA |
| | | GGATTACAAGCAGCAATTTACCTGGAAGTGATA |
| | | CCTTAGGGGCAATCTTGAAGATACACTTTCCTGA |
| | | AAAATGATTTGTGATGGATTGTATATTTATTTAA |
| | | AATATCTTGGGAGGGGAGGCTGATGGAGATAGG |
| | | GAGCATGCTCAAACCTCCCTAAGACAAGCTGCT |
| | | GCTGTGACTATGGGCTCCCAAAGAGCTAGATCG |
| | | TATATTTATTTGACAAAAATCACCATAGACTGCA |
| | | TCCATACTACAGAGAAAAAACAATTAGGGCGCA |
| | | AATGGATAGTTACAGTAAAGTCTTCAGCAAGCA |
| | | GCTGCCTGTATTCTAAGCACTGGGATTTTCTGTT |
| | | TCGTGCAAATATTTATCTCATTATTGTTGTGATC |
| | | TAGTTCAATAACCTAGAATTTGAATTGTCACCAC |
| | | ATAGCTTTCAATCTGTGCCAACAACTATACAATT |
| | | CATCAAGTGTG |
| Antibody 2 | | |
| HCDR1 (Kabat) | 3 | TAAMS |
| HCDR2 (Kabat) | 4 | GISGSGSSTYYADSVKG |
| HCDR3 (Kabat) | 5 | ELSYLYSGYYFDY |
| HCDR1 (Chothia) | 6 | GFTFSTA |
| HCDR2 (Chothia) | 7 | SGSGSS |
| HCDR3 (Chothia) | 8 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 9 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 10 | CAGGTGCAATTGCTGGAAAGCGGCGGTGGCCTG |
| | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| Heavy Chain | 11 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPEAAGG |
| | | PSVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCKVSNKALPAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 12 | CAGGTGCAATTGCTGGAAAGCGGCGGTGGCCTG |
| Chain | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| | | GCCTCCACCAAGGGTCCATCGGTCTTCCCCCTGG |
| | | CACCCTCCTCCAAGAGCACCTCTGGGGGCACAG |
| | | CGGCCCTGGGCTGCCTGGTCAAGGACTACTTCC |
| | | CCGAACCGGTGACGGTGTCGTGGAACTCAGGCG |
| | | CCCTGACCAGCGGCGTGCACACCTTCCCGGCTG |
| | | TCCTACAGTCCTCAGGACTCTACTCCCTCAGCAG |
| | | CGTGGTGACCGTGCCCTCCAGCAGCTTGGGCAC |
| | | CCAGACCTACATCTGCAACGTGAATCACAAGCC |
| | | CAGCAACACCAAGGTGGACAAGAGAGTTGAGC |
| | | CCAAATCTTGTGACAAAACTCACACATGCCCAC |
| | | CGTGCCCAGCACCTGAAGCAGCGGGGGGACCGT |
| | | CAGTCTTCCTCTTCCCCCCAAAACCCAAGGACAC |
| | | CCTCATGATCTCCCGGACCCCTGAGGTCACATGC |
| | | GTGGTGGTGGACGTGAGCCACGAAGACCCTGAG |
| | | GTCAAGTTCAACTGGTACGTGGACGGCGTGGAG |
| | | GTGCATAATGCCAAGACAAAGCCGCGGGAGGA |
| | | GCAGTACAACAGCACGTACCGGGTGGTCAGCGT |
| | | CCTCACCGTCCTGCACCAGGACTGGCTGAATGG |
| | | CAAGGAGTACAAGTGCAAGGTCTCCAACAAAGC |
| | | CCTCCCAGCCCCCATCGAGAAAACCATCTCCAA |
| | | AGCCAAAGGGCAGCCCCGAGAACCACAGGTGT |
| | | ACACCCTGCCCCCATCCCGGGAGGAGATGACCA |
| | | AGAACCAGGTCAGCCTGACCTGCCTGGTCAAAG |
| | | GCTTCTATCCCAGCGACATCGCCGTGGAGTGGG |
| | | AGAGCAATGGGCAGCCGGAGAACAACTACAAG |
| | | ACCACGCCTCCCGTGCTGGACTCCGACGGCTCCT |
| | | TCTTCCTCTACAGCAAGCTCACCGTGGACAAGA |
| | | GCAGGTGGCAGCAGGGGAACGTCTTCTCATGCT |
| | | CCGTGATGCATGAGGCTCTGCACAACCACTACA |
| | | CGCAGAAGAGCCTCTCCCTGTCTCCGGGTAAA |
| LCDR1 (Kabat) | 13 | SGSSSNIGSNDVS |
| LCDR2 (Kabat) | 14 | KNYNRPS |
| LCDR3 (Kabat) | 15 | SAWDQRQFDVV |
| LCDR1 (Chothia) | 16 | SSSNIGSND |
| LCDR2 (Chothia) | | KNY |
| LCDR3 (Chothia) | 18 | WDQRQFDV |
| LCDR1 (IMGT) | 47 | SSNIGSND |
| LCDR2 (IMGT) | | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 19 | DIVLTQPPSVSGAPGQRVTISCSGSSSNIGSNDVSW |
| | | YQQLPGTAPKLLIYKNYNRPSGVPDRFSGSKSGTS |
| | | ASLAITGLQAEDEADYYCSAWDQRQFDVVFGGGT |
| | | KLTVL |
| DNA encoding VL | 20 | GATATCGTGCTGACCCAGCCGCCGAGCGTGAGC |
| | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTA |
| Light Chain | 21 | DIVLTQPPSVSGAPGQRVTISCSGSSSNIGSNDVSW |
| | | YQQLPGTAPKLLIYKNYNRPSGVPDRFSGSKSGTS |
| | | ASLAITGLQAEDEADYYCSAWDQRQFDVVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 22 | GATATCGTGCTGACCCAGCCGCCGAGCGTGAGC |
| Chain | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTAG |
| | | GTCAGCCCAAGGCTGCCCCCTCGGTCACTCTGTT |
| | | CCCGCCCTCCTCTGAGGAGCTTCAAGCCAACAA |
| | | GGCCACACTGGTGTGTCTCATAAGTGACTTCTAC |
| | | CCGGGAGCCGTGACAGTGGCCTGGAAGGCAGAT |
| | | AGCAGCCCCGTCAAGGCGGGAGTGGAGACCACC |
| | | ACACCCTCCAAACAAAGCAACAACAAGTACGCG |
| | | GCCAGCAGCTATCTGAGCCTGACGCCTGAGCAG |
| | | TGGAAGTCCCACAGAAGCTACAGCTGCCAGGTC |
| | | ACGCATGAAGGGAGCACCGTGGAGAAGACAGT |
| | | GGCCCCTACAGAATGTTCA |
| Antibody 1 | | |
| HCDR1 (Kabat) | 23 | TAAMS |
| HCDR2 (Kabat) | 24 | GISGSGSSTYYADSVKG |
| HCDR3 (Kabat) | 25 | ELSYLYSGYYFDY |
| HCDR1 (Chothia) | 26 | GFTFSTA |
| HCDR2 (Chothia) | 27 | SGSGSS |
| HCDR3 (Chothia) | 28 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 29 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 30 | CAGGTGCAGCTGCTGGAATCAGGCGGCGGACTG |
| | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | C |
| Heavy Chain | 31 | QVQLLESGGGLVQPGGSLRLSCAASGFTFSTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPELLGGP |
| | | SVFLFPPKPKDTLMISRTPEVTCVVVAVSHEDPEV |
| | | KFNWYVDGVEVHNAKTKPREEQYNSTYRVVSVL |
| | | TVLHQDWLNGKEYKCKVSNKALAAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 32 | CAGGTGCAGCTGCTGGAATCAGGCGGCGGACTG |
| Chain | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | CGCTAGCACTAAGGGCCCCTCCGTGTTCCCTCTG |
| | | GCCCCTTCCAGCAAGTCTACCTCCGGCGGCACA |
| | | GCTGCTCTGGGCTGCCTGGTCAAGGACTACTTCC |
| | | CTGAGCCTGTGACAGTGTCCTGGAACTCTGGCG |
| | | CCCTGACCTCTGGCGTGCACACCTTCCCTGCCGT |
| | | GCTGCAGTCCTCCGGCCTGTACTCCCTGTCCTCC |
| | | GTGGTCACAGTGCCTTCAAGCAGCCTGGGCACC |
| | | CAGACCTATATCTGCAACGTGAACCACAAGCCT |
| | | TCCAACACCAAGGTGGACAAGCGGGTGGAGCCT |
| | | AAGTCCTGCGACAAGACCCACACCTGTCCTCCC |
| | | TGCCCTGCTCCTGAACTGCTGGGCGGCCCTTCTG |
| | | TGTTCCTGTTCCCTCCAAAGCCCAAGGACACCCT |
| | | GATGATCTCCCGGACCCCTGAAGTGACCTGCGT |
| | | GGTGGTGGCCGTGTCCCACGAGGATCCTGAAGT |
| | | GAAGTTCAATTGGTACGTGGACGGCGTGGAGGT |
| | | GCACAACGCCAAGACCAAGCCTCGGGAGGAAC |
| | | AGTACAACTCCACCTACCGGGTGGTGTCCGTGC |
| | | TGACCGTGCTGCACCAGGACTGGCTGAACGGCA |
| | | AAGAGTACAAGTGCAAAGTCTCCAACAAGGCCC |
| | | TGGCCGCCCCTATCGAAAAGACAATCTCCAAGG |
| | | CCAAGGGCCAGCCTAGGGAACCCCAGGTGTACA |
| | | CCCTGCCACCCAGCCGGGAGGAAATGACCAAGA |
| | | ACCAGGTGTCCCTGACCTGTCTGGTCAAGGGCTT |
| | | CTACCCTTCCGATATCGCCGTGGAGTGGGAGTCT |
| | | AACGGCCAGCCTGAGAACAACTACAAGACCACC |
| | | CCTCCTGTGCTGGACTCCGACGGCTCCTTCTTCC |
| | | TGTACTCCAAACTGACCGTGGACAAGTCCCGGT |
| | | GGCAGCAGGGCAACGTGTTCTCCTGCTCCGTGA |
| | | TGCACGAGGCCCTGCACAACCACTACACCCAGA |
| | | AGTCCCTGTCCCTGTCTCCCGGCAAG |
| LCDR1 (Kabat) | 33 | SGSSSNIGSNDVS |
| LCDR2 (Kabat) | 34 | KNYNRPS |
| LCDR3 (Kabat) | 35 | SAWDQRQFDVV |
| LCDR1 (Chothia) | 36 | SSSNIGSND |
| LCDR2 (Chothia) | | KNY |
| LCDR3 (Chothia) | 38 | WDQRQFDV |
| LCDR1 (IMGT) | 47 | SSNIGSND |
| LCDR2 (IMGT) | | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 39 | QSVLTQPPSASGTPGQRVTISCSGSSSNIGSNDVSW |
| | | YQQLPGTAPKLLIYKNYNRPSGVPDRFSGSKSGTS |
| | | ASLAISGLQSEDEADYYCSAWDQRQFDVVFGGGT |
| | | KLTVL |
| DNA encoding VL | 40 | CAGTCAGTCCTGACTCAGCCCCCTAGCGCTAGT |
| | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| Light Chain | 41 | QSVLTQPPSASGTPGQRVTISCSGSSSNIGSNDVSW |
| | | YQQLPGTAPKLLIYKNYNRPSGVPDRFSGSKSGTS |
| | | ASLAISGLQSEDEADYYCSAWDQRQFDVVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 42 | CAGTCAGTCCTGACTCAGCCCCCTAGCGCTAGT |
| Chain | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| | | GGTCAACCTAAGGCTGCCCCCAGCGTGACCCTG |
| | | TTCCCCCCCAGCAGCGAGGAGCTGCAGGCCAAC |
| | | AAGGCCACCCTGGTGTGCCTGATCAGCGACTTC |
| | | TACCCAGGCGCCGTGACCGTGGCCTGGAAGGCC |
| | | GACAGCAGCCCCGTGAAGGCCGGCGTGGAGACC |
| | | ACCACCCCCAGCAAGCAGAGCAACAACAAGTAC |
| | | GCCGCCAGCAGCTACCTGAGCCTGACCCCCGAG |
| | | CAGTGGAAGAGCCACAGGTCCTACAGCTGCCAG |
| | | GTGACCCACGAGGGCAGCACCGTGGAAAAGAC |
| | | CGTGGCCCCAACCGAGTGCAGC |

In some embodiments, other antibodies for use in the methods or formulations described herein (e.g., methods for treating a subject afflicted with or at risk of developing a thromboembolic disorder) include those where the amino acids or nucleic acids encoding the amino acids have been mutated, yet have at least 60, 65, 70, 75, 80, 85, 90, or 95 percent identity to the sequences described in Table 1. Some embodiments include mutant amino acid sequences wherein no more than 1, 2, 3, 4 or 5 amino acids have been mutated in the variable regions when compared with the variable regions depicted in the sequence described in Table 1, while retaining substantially the same antigen binding activity.

Since each of these antibodies can bind to FXI and/or FXIa, the VH, VL, full length light chain, and full length heavy chain sequences (amino acid sequences and the nucleotide sequences encoding the amino acid sequences) can be “mixed and matched” to create other FXI and/or FXIa-binding antibodies of the present disclosure. Such “mixed and matched” FXI and/or FXIa-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

Accordingly, in one aspect, for use in the methods described herein (e.g., methods for treating a subject afflicted with or at risk of developing a thromboembolic disorder), the present disclosure provides an isolated antibody or antigen binding region thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa). In another aspect, for use in the formulations described herein (e.g., the formulation in the vial, the intravenous drug delivery formulation), the present disclosure provides an isolated antibody or antigen binding region thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa).

More specifically, in certain aspects, the present disclosure provides an isolated antibody or antigen binding fragment thereof having a heavy chain variable domain and a light chain variable domain comprising amino acid sequences selected from SEQ ID NOs: 9 and 29; or 19 and 39, respectively.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39.

In another aspect for use in the methods described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding region thereof having a heavy chain and a light chain comprising amino acid sequences selected from SEQ ID NOs: 11 and 31; or 21 and 41, respectively. In another aspect for use in the formulations described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding region thereof having a heavy chain and a light chain comprising amino acid sequences selected from SEQ ID NOs: 11 and 31; or 21 and 41, respectively.

In a specific embodiment for use in the methods described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21. In a specific embodiment for use in the formulations described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21.

In a specific embodiment for use in the methods described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41. In a specific embodiment for use in the formulations described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41.

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme), Lefranc et al., (2003) Dev. Comp. Immunol., 27, 55-77 (“IMGT” numbering scheme), or the “Combined” system.

For example, under Kabat, the CDR amino acid residues of Antibody 2 in the heavy chain variable domain (VH) are numbered 31-35 (HCDR1), 50-66 (HCDR2), and 99-111 (HCDR3); and the CDR amino acid residues in the light chain variable domain (VL) are numbered 22-35 (LCDR1), 51-57 (LCDR2), and 90-100 (LCDR3). Under Chothia the CDR amino acids in the VH are numbered 26-32 (HCDR1), 52-57 (HCDR2), and 99-111 (HCDR3); and the amino acid residues in VL are numbered 25-33 (LCDR1), 51-53 (LCDR2), and 92-99 (LCDR3). By combining the CDR definitions of both Kabat and Chothia, the CDRs consist of amino acid residues 26-35 (HCDR1), 50-66 (HCDR2), and 99-111 (HCDR3) in human VH and amino acid residues 22-35 (LCDR1), 51-57 (LCDR2), and 90-100 (LCDR3) in human VL. By combining the CDR definitions of both Kabat and Chothia, the “Combined” CDRs consist of amino acid residues 26-35 (HCDR1), 50-66 (HCDR2), and 99-108 (HCDR3) in human VH and amino acid residues 24-38 (LCDR1), 54-60 (LCDR2), and 93-101 (LCDR3) in human VL. As another example, under IMGT, the CDR amino acid residues in the heavy chain variable domain (VH) are numbered 26-33 (HCDR1), 51-58 (HCDR2), and 97-108 (HCDR3); and the CDR amino acid residues in the light chain variable domain (VL) are numbered 27-36 (LCDR1), 54-56 (LCDR2), and 93-101 (LCDR3). Table 1 provides exemplary Kabat, Chothia, Combined, and IMGT HCDR1, HCDR2, HCDR3, LCDR1, LCDR2, and LCDR3 for anti-FXI/FXIa antibodies, e.g., Antibody 2 and Antibody 1. In another aspect, the present disclosure provides FXIa binding antibodies that comprise the heavy chain and light chain CDR1s, CDR2s, and CDR3s as described in Table 1, or combinations thereof. The amino acid sequences of the VH CDR Is of the antibodies are shown in SEQ ID NOs: 3 and 23. The amino acid sequences of the VH CDR2s of the antibodies are shown in SEQ ID NOs: 4 and 24. The amino acid sequences of the VH CDR3s of the antibodies are shown in SEQ ID NOs: 5 and 25. The amino acid sequences of the VL CDR1s of the antibodies are shown in SEQ ID NOs: 13 and 33. The amino acid sequences of the VL CDR2s of the antibodies are shown in SEQ ID NOs: 14 and 34. The amino acid sequences of the VL CDR3s of the antibodies are shown in SEQ ID NOs: 15 and 35. These CDR regions are delineated using the Kabat system.

Alternatively, as defined using the Chothia system (Al-Lazikani et al., (1997) JMB 273, 927-948), the amino acid sequences of the VH CDR1s of the antibodies are shown in SEQ ID NOs: 6 and 26. The amino acid sequences of the VH CDR2s of the antibodies and are shown in SEQ ID NOs: 7 and 27. The amino acid sequences of the VH CDR3s of the antibodies are shown in SEQ ID NOs: 8 and 28. The amino acid sequences of the VL CDR1s of the antibodies are shown in SEQ ID NOs: 16 and 36. The amino acid sequences of the VL CDR2s of the antibodies is KNY. The amino acid sequences of the VL CDR3s of the antibodies are shown in SEQ ID NOs: 18 and 38.

Alternatively, as defined using the Combined system, the amino acid sequences of the VH CDR1 of the antibodies are shown in SEQ ID NO: 46. The amino acid sequences of the VH CDR2 of the antibodies and are shown in SEQ ID NO: 4. The amino acid sequences of the VH CDR3 of the antibodies are shown in SEQ ID NO: 5. The amino acid sequences of the VL CDR1 of the antibodies are shown in SEQ ID NO: 33. The amino acid sequences of the VL CDR2 of the antibodies are shown in SEQ ID NO: 14. The amino acid sequences of the VL CDR3 of the antibodies are shown in SEQ ID NO: 15.

Alternatively, as defined using the IMGT numbering scheme, the amino acid sequences of the VH CDR1 of the antibodies are shown in SEQ ID NO: 43. The amino acid sequences of the VH CDR2 of the antibodies and are shown in SEQ ID NO: 44. The amino acid sequences of the VH CDR3 of the antibodies are shown in SEQ ID NO: 45. The amino acid sequences of the VL CDR1 of the antibodies are shown in SEQ ID NO: 47. The amino acid sequences of the VL CDR2 of the antibodies is KNY. The amino acid sequences of the VL CDR3 of the antibodies are shown in SEQ ID NO: 15.

Given that each of these antibodies can bind to FXI and/or FXIa and that antigen-binding specificity is provided primarily by the CDR1, 2 and 3 regions, the VH CDR1, 2 and 3 sequences and VL CDR1, 2 and 3 sequences can be “mixed and matched” (e.g., CDRs from different antibodies can be mixed and matched, although each antibody preferably contains a VH CDR1, 2 and 3 and a VL CDR1, 2 and 3 to create other FXI and/or FXIa binding molecules of the present disclosure). Such “mixed and matched” FXI and/or FXIa binding antibodies can be tested using the binding assays known in the art and those described in the Examples (e.g., ELISAs, SET, BIACORE™ assays). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure. In addition to the foregoing, in one embodiment, the antigen binding fragments of the antibodies described herein can comprise a VH CDR1, 2, and 3, or a VL CDR 1, 2, and 3, wherein the fragment binds to FXI and/or FXIa as a single variable domain. It is noted that the CDR sequences of Antibody 1 and Antibody 2 are identical.

In certain embodiments of the present disclosure, the antibodies or antigen binding fragments thereof may have the heavy and light chain sequences of the Fabs described in Table 1. More specifically, the antibody or antigen binding fragments thereof may have the heavy and light sequence of Antibody 2 and Antibody 1.

In other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Kabat and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Chothia and described in Table 1. In other embodiments, the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by the Combined system and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by IMGT and described in Table 1.

In a specific embodiment for use in the methods described herein, the present disclosure includes an antibody that specifically binds to FXI and/or FXIa comprising a heavy chain variable region CDR1 of SEQ ID NO: 3; a heavy chain variable region CDR2 of SEQ ID NO: 4; a heavy chain variable region CDR3 of SEQ ID NO: 5; a light chain variable region CDR1 of SEQ ID NO: 13; a light chain variable region CDR2 of SEQ ID NO: 14; and a light chain variable region CDR3 of SEQ ID NO: 15.

In a specific embodiment, the present disclosure includes an antibody that specifically binds to FXI and/or FXIa comprising a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35.

In a specific embodiment, the present disclosure includes an antibody that specifically binds to FXI and/or FXIa comprising a heavy chain variable region CDR1 of SEQ ID NO: 6; a heavy chain variable region CDR2 of SEQ ID NO: 7; a heavy chain variable region CDR3 of SEQ ID NO: 8; a light chain variable region CDR1 of SEQ ID NO: 16; a light chain variable region CDR2 of KNY; and a light chain variable region CDR3 of SEQ ID NO: 18.

In a specific embodiment, the present disclosure includes an antibody that specifically binds to FXI and/or FXIa comprising a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY; and a light chain variable region CDR3 of SEQ ID NO: 38.

In a specific embodiment, provided herein is an antibody that specifically binds to FXI and/or FXIa comprising a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY and a light chain variable region CDR3 of SEQ ID NO: 15.

In a specific embodiment, provided herein is an antibody that specifically binds to FXI and/or FXIa comprising a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14 and a light chain variable region CDR3 of SEQ ID NO: 15.

In certain embodiments, the present disclosure includes antibodies or antigen binding fragments that specifically bind to FXI and/or FXIa as described in Table 1. In a specific embodiment for use in the methods described herein, the antibody, or antigen binding fragment, that binds FXI and/or FXIa is Antibody 2 and Antibody 1.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described below, as well as DP47 and DPK9.

### Homologous Antibodies

In yet other embodiments for use in the methods described herein (e.g., methods for treating a subject afflicted with or at risk of developing a thromboembolic disorder), the present disclosure provides an antibody, or an antigen binding fragment thereof, comprising amino acid sequences that are homologous to the sequences described in Table 1 (e.g., SEQ ID NOs: 29, 31, 39, or 41), and the antibody binds to a FXI and/or FXIa protein (e.g., human, rabbit, cynomolgus monkey, and baboon FXIa), and retains the desired functional properties of those antibodies described in Table 1 such as Antibody 2 and Antibody 1. In specific aspects, such homologous antibodies retain the CDR amino acid sequences described in Table 1 (e.g., Kabat CDRs, Chothia CDRs, IMGT CDRs, or Combined CDRs).

For example, in some embodiments the present disclosure provides an isolated antibody, or a functional antigen binding fragment thereof, comprising a heavy chain variable domain and a light chain variable domain, wherein the heavy chain variable domain comprises an amino acid sequence that is at least 80%, at least 90%, or at least 95% identical to an amino acid sequence selected from the group consisting of SEQ ID NOs: 9 and 29; the light chain variable domain comprises an amino acid sequence that is at least 80%, at least 90%, or at least 95% identical to an amino acid sequence selected from the group consisting of SEQ ID NOs: 19 and 39; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 9; the light chain variable domain comprises an amino acid sequence that is at least 80%, at least 90%, or at least 95% identical to the amino acid sequence of SEQ ID NO: 19; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 29; the light chain variable domain comprises an amino acid sequence that is at least 80%, at least 90%, or at least 95% identical to the amino acid sequence of SEQ ID NO: 39; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, KNY, and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, KNY, and 15, respectively.

In other embodiments for use in the methods described herein, the VH and/or VL amino acid sequences may be 50%, 60%, 70%, 80%, 90%, 95%, 96%, 97%, 98% or 99% identical to the sequences set forth in Table 1. In other embodiments for use in the formulations described herein, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except for an amino acid substitution in no more than 1, 2, 3, 4 or 5 amino acid positions. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 10 or 30 and SEQ ID NOs: 20 and 40, respectively, followed by testing of the encoded altered antibody for retained function using the functional assays described herein.

In other embodiments for use in the methods described herein, the full length heavy chain and/or full length light chain amino acid sequences may be 50%60%, 70%, 80%, 90%, 95%, 96%, 97%, 98% or 99% identical to the sequences set forth in Table 1 (e.g., SEQ ID NOs: 11 and/or 21, or 31 and/or 41). In other embodiments for use in the formulations described herein, the full length heavy chain and/or full length light chain amino acid sequences may be 50%60%, 70%, 80%, 90%, 95%, 96%, 97%, 98% or 99% identical to the sequences set forth in Table 1 (e.g., SEQ ID NOs: 11 and/or 21, or 31 and/or 41). An antibody having a full length heavy chain and full length light chain having high (e.g., 80% or greater) identity to the full length heavy chains of any of SEQ ID NOs: 11 or 31, and full length light chains of any of SEQ ID NOs: 21 or 41, can be obtained by mutagenesis (e.g., site-directed or PCR-mediated mutagenesis) of nucleic acid molecules encoding such polypeptides, followed by testing of the encoded altered antibody for retained function using the functional assays described herein.

In one aspect, provided herein is an isolated antibody, or a functional antigen binding fragment thereof, comprising a heavy chain and a light chain, wherein the heavy chain comprises an amino acid sequence that is at least 80%, at least 90%, or at least 95% identical to an amino acid sequence selected from the group consisting of SEQ ID NOs: 11 and 31; the light chain comprises an amino acid sequence that is at least 80%, at least 90%, or at least 95% identical to an amino acid sequence selected from the group consisting of SEQ ID NOs: 21 and 41; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 11; the light chain comprises an amino acid sequence that is at least 80%, at least 90%, or at least 95% identical to the amino acid sequence of SEQ ID NO: 21; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 31; the light chain comprises an amino acid sequence that is at least 80%, at least 90%, or at least 95% identical to the amino acid sequence of SEQ ID NO: 41; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, KNY, and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, KNY, and 15, respectively.

In other embodiments for use in the methods described herein, the full length heavy chain and/or full length light chain nucleotide sequences may be 60%, 70%, 80%, 90%, 95%, 96%, 97%, 98% or 99% identical to the sequences set forth in Table 1 (e.g., SEQ ID NOs: 12 and/or 22, or 32 and/or 42).

In other embodiments for use in the methods described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40). In other embodiments for use in the formulations described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40).

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The isolated anti-FXI and/or FXIa antibodies, or antigen binding fragments thereof, as described herein can be monoclonal antibodies, human or humanized antibodies, chimeric antibodies, single chain antibodies, Fab fragments, Fv fragments, F(ab′)2 fragments, or scFv fragments, and/or IgG isotypes (e.g., IgG1 such as human IgG1). In specific embodiments, anti-FXI and/or anti-FXIa antibodies described herein are recombinant human antibodies. In specific embodiments, anti-FXI and/or anti-FXIa antibodies described herein are human IgG1/lambda (2) antibodies. In specific embodiments, anti-FXI and/or anti-FXIa antibodies described herein are human IgG1/lambda (2) antibodies comprising an Fc domain engineered to reduce the potential for effector function (e.g., ADCC and/or CDC), for example a human Fc domain comprising D265A and/or P329A substitutions.

Additionally or alternatively, the protein sequences of the present disclosure can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain other embodiments, an antibody of the present disclosure for use in the methods described herein (e.g., methods for treating a subject afflicted with or at risk of developing a thromboembolic disorder) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure. In certain other embodiments, an antibody of the present disclosure for use in the formulations described herein (e.g., the formulation in the vial, the intravenous drug delivery formulation) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure.

Accordingly, for use in the methods described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

For use in the formulations described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

In one aspect, provided herein is an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of those described in Table 1, and conservative modifications thereof; the heavy chain variable region CDR2 amino acid sequences are selected from the group consisting of those described in Table 1, and conservative modifications thereof; the heavy chain variable region CDR3 amino acid sequences are selected from the group consisting of those described in Table 1, and conservative modifications thereof; the light chain variable regions CDR1 amino acid sequences are selected from the group consisting of those described in Table 1, and conservative modifications thereof; the light chain variable regions CDR2 amino acid sequences are selected from the group consisting of those described in Table 1, and conservative modifications thereof; the light chain variable regions of CDR3 amino acid sequences are selected from the group consisting of those described in Table 1, and conservative modifications thereof; and the antibody or antigen binding fragments thereof specifically binds to FXIa.

In other embodiments for use in the methods described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. In other embodiments for use in the formulations described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. Accordingly, the present disclosure provides an isolated antibody optimized for expression in a mammalian cell consisting of a full length heavy chain and a full length light chain wherein the full length heavy chain has amino acid sequences selected from the group of SEQ ID NOs: 11 or 31, and conservative modifications thereof; and the full length light chain has amino acid sequences selected from the group of SEQ ID NOs: 21 or 41, and conservative modifications thereof; and the antibody specifically binds to FXI and/or FXIa (e.g., human, rabbit, cynomolgus monkey, and baboon FXIa).

Antibodies that Bind to the Same Epitope

In some embodiments, the present disclosure provides antibodies that compete for the same epitope as the FXI and/or FXIa binding antibodies described in Table 1, for use in the methods described herein (e.g., methods for treating a subject afflicted with or at risk of developing a thromboembolic disorder). In some embodiments, the present disclosure provides antibodies that compete for the same epitope as the FXI and/or FXIa binding antibodies described in Table 1, for use in the formulations described herein (e.g., the formulation in the vial, the intravenous drug delivery formulation). Additional antibodies can therefore be identified based on their ability to compete (e.g., to competitively inhibit the binding of, in a statistically significant manner, by binding to the same or overlapping epitope) with other antibodies of the present disclosure in FXI and/or FXIa binding assays (such as those described in the Examples Section). The ability of a test antibody to inhibit the binding of antibodies of the present disclosure to a FXI and/or FXIa protein demonstrates that the test antibody can compete with that antibody for binding to FXI and/or FXIa; such an antibody may, according to non-limiting theory, bind to the same or a related (e.g., a structurally similar or spatially proximal) epitope on the FXI and/or FXIa protein as the antibody with which it competes. In a certain embodiment, the antibody that binds to the same epitope on FXI and/or FXIa as the antibodies of the present disclosure is a human monoclonal antibody. Such human monoclonal antibodies can be prepared and isolated as described herein.

As used herein, an antibody “competes” for binding when the competing antibody binds to the same FXI and/or FXIa epitope as an antibody or antigen binding fragment of the present disclosure (e.g., Antibody 1 or Antibody 2) and inhibits FXI and/or FXIa binding of an antibody or antigen binding fragment of the present disclosure by more than 50% (for example, 80%, 85%, 90%, 95%, 98% or 99%) in the presence of an equimolar concentration of competing antibody. This may be determined, for instance, in a competitive binding assay, by any of the methods well known to those of skill in the art.

As used herein, an antibody or antigen binding fragment thereof does not “compete” with a FXI and/or FXIa antibody or antigen binding fragment of the present disclosure (e.g., Antibody 1 or Antibody 2) unless said competing antibody or antigen binding fragment thereof binds the same FXI and/or FXIa epitope, or an overlapping FXI and/or FXIa epitope, as an antibody or antigen binding fragment of the present disclosure. As used herein, a competing antibody or antigen binding fragment thereof does not include one which (i) sterically blocks an antibody or antigen binding fragment of the present disclosure from binding its target (e.g., if said competing antibody binds to a nearby, non-overlapping FXI and/or FXIa epitope and physically prevents an antibody or antigen binding fragment of the present disclosure from binding its target); and/or (ii) binds to a different, non-overlapping FXI and/or FXIa epitope and induces a conformational change to the FXI and/or FXIa protein such that said protein can no longer be bound by a FXI and/or FXIa antibody or antigen binding fragment of the present disclosure in a way that would occur absent said conformational change.

### Engineered and Modified Antibodies

In some embodiments, an antibody of the present disclosure, for use in the methods described herein, further can be prepared using an antibody having one or more of the VH and/or VL sequences shown herein as starting material to engineer a modified antibody, which modified antibody may have altered properties from the starting antibody. In some embodiments, an antibody of the present disclosure, for use in the formulations described herein, further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the present disclosure pertains to an isolated antibody, or an antigen binding fragment thereof, comprising a heavy chain variable region comprising CDR1 sequences having an amino acid sequence selected from the group consisting of SEQ ID NOs: 3 and 23; CDR2 sequences having an amino acid sequence selected from the group consisting of SEQ ID NOs: 4 and 24; CDR3 sequences having an amino acid sequence selected from the group consisting of SEQ ID NOs: 5 and 25, respectively; and a light chain variable region having CDR1 sequences having an amino acid sequence selected from the group consisting of SEQ ID NOs: 13 and 33; CDR2 sequences having an amino acid sequence selected from the group consisting of SEQ ID NOs: 14 and 34; and CDR3 sequences consisting of an amino acid sequence selected from the group consisting of SEQ ID NOs: 15 and 35, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in the antibodies of the present disclosure are those that are structurally similar to the framework sequences used by selected antibodies of the present disclosure, e.g., consensus sequences and/or framework sequences used by monoclonal antibodies of the present disclosure. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, Vl2, and Vk2.

Accordingly, for use in the methods described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Accordingly, for use in the formulations described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Another type of variable region modification is mutation of amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples Section. Conservative modifications (as discussed above) can be introduced. The mutations may be amino acid substitutions, additions or deletions. Moreover, typically no more than one, two, three, four or five residues within a CDR region are altered.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence of KNY, or an amino acid sequence having one, two, three, four or five amino acid substitutions, deletions or additions as compared to KNY; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence of KNY, or an amino acid sequence having one, two, three, four or five amino acid substitutions, deletions or additions as compared to KNY; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Antibodies with Extended Half Life

In some embodiments, the present disclosure provides for antibodies that specifically bind to FXIa protein which have an extended half-life in vivo, for use in the methods or formulations described herein.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half-life of the antibodies of the present disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the present disclosure. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a nonnative amino acid any place an amber codon is positioned, converting the amber from a stop codon to one that signals incorporation of the chemically specified amino acid.

Recombinant pegylation technology (rPEG) can also be used for serum half-life extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to FXIa while a second binding domain of the antibody binds to serum albumin, preferably HSA.

The strategies for increasing half-life is especially useful in nanobodies, fibronectin-based binders, and other antibodies or proteins for which increased in vivo half-life is desired.

### Antibody Conjugates

In some embodiments, the present disclosure provides antibodies or fragments thereof, for use in the methods or formulations described herein, that specifically bind to an FXIa protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the present disclosure provides fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the present disclosure or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2): 76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an FXIa protein may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide (SEQ ID NO: 48), such as the tag provided in a pQE vector (QIAGEN, Inc., 9259 Eton Avenue, Chatsworth, CA, 91311), among others, many of which are commercially available. As described in Gentz et al., 1989, Proc. Natl. Acad. Sci. USA 86:821-824, for instance, hexa-histidine (SEQ ID NO: 48) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present disclosure or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin; and positron emitting metals using various positron emission tomographies, and nonradioactive paramagnetic metal ions.

In some embodiments, the present disclosure further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alpha-emiters such as ²¹³Bi or macrocyclic chelators useful for conjugating radiometal ions, including but not limited to, ¹³¹In, ¹³¹Lu, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Formulations

In some embodiments, the present disclosure also provides pharmaceutical formulations that contain a therapeutically effective amount of a Factor XI and/or Factor XIa antibody disclosed herein (e.g., Antibody 1). The pharmaceutical formulation comprises one or more excipients and is maintained at a certain pH. Non-limiting examples of an “excipient,” as used herein, include any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

### Drug Substance

Antibody 1 is a high-affinity, anti-human Factor XI monoclonal antibody. It is expressed in a Chinese hamster ovary cell line (CHO-C8TD). In some embodiments, the Antibody 1 drug substance is fully formulated for subcutaneous administration (i.e., no further excipients are added), and thus is identical in composition to the Antibody 1 drug product. In some embodiments, for intravenous administration, the Antibody 1 drug product is further diluted in an appropriate carrier. In some embodiments, the Antibody 1 drug product is diluted in a solution comprising dextrose, e.g., dextrose 5% in water (D5W).

### Excipients and pH

In some embodiments the excipients contained in the Antibody 1 drug product are pharmacopeial grade excipients. In some embodiments, the excipients in the Antibody 1 drug product comprise a histidine, a histidine salt, a sugar, and a polysorbate. In some embodiments, the excipients in the Antibody 1 drug product include L-histidine and L-histidine hydrochloride monohydrate (histidine buffer), sucrose, and polysorbate 20. Excipients may be selected for their suitability for intravenous and subcutaneous administration, providing the necessary stabilizing, buffering capacity, and tonicity. The formulation maximizes the stability of the monoclonal antibody product, and may provide a sterile solution suitable for subcutaneous or intravenous administration. In some embodiments, a sugar (e.g., sucrose) acts as a stabilizer. In some embodiments, a histidine (e.g., L-histidine, L-Histidine HCl monohydrate) acts as a buffering agent. In some embodiments, a polysorbate (e.g., polysorbate 20), acts as a stabilizer. In some embodiments, the formulation is adjusted to final volume in water for injection (WFI).

The one or more excipients in the pharmaceutical formulation of the present invention comprises a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system comprises at least one buffer that has a buffering range that overlaps fully or in part with the range of pH 5.0-7.4. In certain embodiments, the buffer has a pH of about 5.5±0.5. In certain embodiments, the buffer comprises a histidine buffer. In certain embodiments, the histidine buffer is present at a concentration of 0.05-10 mM, 0.1-10 mM, 0.2-10 mM, 0.5-10 mM, 1-10 mM, 5-10 mM, 5 to 100 mM, 10 to 100 mM, 15 to 100 mM, 20 to 100 mM, 30 to 100 mM, 40 to 100 mM, 50 to 100 mM, 60 to 100 mM, 70 to 100 mM, 80 to 100 mM, 90 to 100 mM, 5 to 90 mM, 5 to 80 mM, 5 to 70 mM, 5 to 60 mM, 5 to 50 mM, 5 to 40 mM, 5 to 30 mM, 5 to 20 mM, 10 to 50 mM, 10 to 40 mM, 10 to 30 mM, 10 to 20 mM, 5 to 25 mM, 10 to 25 mM, 15 to 25 mM, 20 to 25 mM, 5 to 20 mM, 10 to 20 mM, or 15 to 20 mM. In certain embodiments, the histidine is present at a concentration of about 0.1 mM, 0.2 mM, 0.5 mM, 1 mM, 5 mM, about 10 mM, about 15 mM, about 20 mM, about 25 mM, or about 50 mM. In certain embodiments, the histidine buffer is present at a concentration of about 20 mM. In certain embodiments, the histidine buffer is present at a concentration of about 0.20 mM. In certain embodiments, the histidine buffer has a pH of about 5.0, about 5.5, about 6.0, about 6.5, or about 7.0. In a particular embodiment, the histidine buffer has a pH of about 5.5.

The pharmaceutical formulation of the present invention may have a pH of 5.0 to 6.0. For example, in certain embodiments, the pharmaceutical formulation has a pH of 5.0 to 6.0 (i.e., 5.5±0.5), 5.1 to 5.9 (i.e., 5.5±0.4), 5.2 to 5.8 (i.e., 5.5±0.3), 5.3 to 5.7 (i.e., 5.5±0.2), 5.4 to 5.6 (i.e., 5.5±0.1), or 5.45 to 5.55 (i.e., 5.5±0.05). In certain embodiments, the pharmaceutical formulation has a pH of about 5.5, about 5.6, about 5.7, about 5.8, about 5.9, about 6.0, about 6.1, about 6.2, about 6.3, about 6.4, or about 6.5. In certain embodiments, the pharmaceutical formulation has a pH of about 5.5. Under the rules of scientific rounding, a pH greater than or equal to 5.45 and smaller than or equal to 5.55 is rounded as 5.5.

In certain embodiments, the buffer system of the pharmaceutical formulation comprises histidine at 10 to 30 mM, at a pH of 5.5±0.2. In certain embodiments, the buffer system of the pharmaceutical formulation comprises histidine at about 20 mM, at a pH of 5.5±0.2. In certain embodiments, the buffer system of the pharmaceutical formulation comprises histidine at 10 to 30 mM, at a pH of 5.5±0.05. In certain embodiments, the buffer system of the pharmaceutical formulation comprises histidine at about 20 mM, at a pH of 5.5±0.05.

In certain embodiments, the buffer system of the pharmaceutical formulation comprises histidine at 0.10 to 0.30 mM, at a pH of 5.5±0.2. In certain embodiments, the buffer system of the pharmaceutical formulation comprises histidine at about 0.20 mM, at a pH of 5.5±0.2. In certain embodiments, the buffer system of the pharmaceutical formulation comprises histidine at 0.10 to 0.30 mM, at a pH of 5.5±0.05. In certain embodiments, the buffer system of the pharmaceutical formulation comprises histidine at about 0.20 mM, at a pH of 5.5±0.05.

The one or more excipients in the pharmaceutical formulation of the present invention further comprises a sugar or sugar alcohol. Sugars and sugar alcohols are useful in pharmaceutical formulations as a thermal stabilizer. In certain embodiments, the pharmaceutical formulation comprises a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation comprises sucrose. In certain embodiments, the pharmaceutical composition comprises a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In specific embodiments, the pharmaceutical formulation comprises sucrose.

The amount of the sugar or sugar alcohol contained within the formulation can vary depending on the specific circumstances and intended purposes for which the formulation is used. In certain embodiments, the pharmaceutical formulation comprises 50 to 300 mM, 50 to 250 mM, 100 to 300 mM, 100 to 250 mM, 150 to 300 mM, 150 to 250 mM, 200 to 300 mM, 200 to 250 mM, or 250 to 300 mM of the sugar or sugar alcohol. In certain embodiments, the pharmaceutical formulation comprises about 50 mM, about 75 mM, about 100 mM, about 125 mM, about 150 mM, about 200 mM, about 220 mM, about 250 mM, or about 300 mM of the sugar or sugar alcohol. In specific embodiments, the pharmaceutical formulation comprises about 220 mM of the sugar or sugar alcohol (e.g., sucrose).

The amount of the sugar or sugar alcohol contained within the formulation can vary depending on the specific circumstances and intended purposes for which the formulation is used. In certain embodiments, the pharmaceutical formulation comprises 0.50 to 3.00 mM, 0.50 to 2.50 mM, 1.00 to 3.00 mM, 1.00 to 2.50 mM, 1.50 to 3.00 mM, 1.50 to 2.50 mM, 2.00 to 3.00 mM, 2.00 to 2.50 mM, or 2.50 to 3.00 mM of the sugar or sugar alcohol. In certain embodiments, the pharmaceutical formulation comprises about 0.50 mM, about 0.75 mM, about 1.00 mM, about 1.25 mM, about 1.50 mM, about 2.00 mM, about 2.20 mM, about 2.50 mM, or about 3.00 mM of the sugar or sugar alcohol. In specific embodiments, the pharmaceutical formulation comprises about 2.20 mM of the sugar or sugar alcohol (e.g., sucrose).

The one or more excipients in the pharmaceutical formulation disclosed herein further comprises a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 20.

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.02% to 0.06%, 0.03% to 0.05%, or 0.035% to 0.045% of the non-ionic surfactant (e.g., polysorbate 20). In certain embodiments, the pharmaceutical formulation comprises about 0.005%, about 0.01%, about 0.02%, about 0.03%, about 0.04%, about 0.05%, about 0.06%, about 0.07%, about 0.08%, about 0.09%, or about 0.1% of the non-ionic surfactant (e.g., polysorbate 20).

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.0002% to 0.0006%, 0.0003% to 0.0005%, or 0.00035% to 0.00045% of the non-ionic surfactant (e.g., polysorbate 20). In certain embodiments, the pharmaceutical formulation comprises about 0.00005%, about 0.0001%, about 0.0002%, about 0.0003%, about 0.0004%, about 0.0005%, about 0.0006%, about 0.0007%, about 0.0008%, about 0.0009%, or about 0.001% of the non-ionic surfactant (e.g., polysorbate 20).

In certain embodiments, the drug product is diluted in an aqueous carrier suitable for the route of administration, e.g., intravenous administration. Exemplary carriers include sterile water for injection (SWFI), bacteriostatic water for injection (BWFI), a pH buffered solution (e.g., phosphate-buffered saline), sterile saline solution, Ringer's solution, or dextrose solution. In one embodiment, when the pharmaceutical formulation is prepared for intravenous administration, the pharmaceutical formulation can be diluted in a 5% dextrose solution (D5W).

### Exemplary Formulations

In certain embodiments, the pharmaceutical formulation of the present invention comprises an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), histidine buffer, a sugar or sugar alcohol (e.g., sucrose), and a polysorbate (e.g., polysorbate 20), at pH 5.5 to 6.5.

In certain embodiments, the pharmaceutical formulation comprises 100 to 200 mg/mL of an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of a sugar or sugar alcohol (e.g., sucrose), and 0.02% to 0.06% of a polysorbate (e.g., polysorbate 20), at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.0 to 6.0. In certain embodiments, the pharmaceutical formulation comprises 100 to 200 mg/mL of the Factor XI and/or Factor XIa antibody, about 20 mM of histidine buffer, about 220 mM of a sugar or sugar alcohol (e.g., sucrose), and about 0.04% of a polysorbate (e.g., polysorbate 20), at pH 5.2 to 5.8. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of a sugar or sugar alcohol (e.g., sucrose), and 0.0002% to 0.0006% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.2 to 5.8. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.45 to 5.55.

In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of sucrose, and 0.02% to 0.06% of polysorbate 20, at pH 5.0 to 6.0. In certain embodiments, the pharmaceutical formulation comprises 100 to 200 mg/mL of the Factor XI and/or Factor XIa antibody, about 20 mM of histidine buffer, about 220 mM of sucrose, and about 0.04% of polysorbate 20, at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.3 to 5.7. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of sucrose, and 0.0002% to 0.0006% of polysorbate 20, at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.0 to 6.0. In certain embodiments, the pharmaceutical formulation comprises 1.00 to 2.00 mg/ml of the Factor XI and/or Factor XIa antibody, 20 mM of histidine buffer, about 2.20 mM of sucrose, and about 0.0004% of polysorbate 20, at pH 5.3 to 5.7. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.45 to 5.55.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

### Stability of the Factor XI and or Factor XIa Antibody

The pharmaceutical formulations of the present invention exhibit high levels of stability. A pharmaceutical formulation is stable when the Factor XI and/or Factor XIa antibody within the formulation retains an acceptable degree of physical property, chemical structure, and/or biological function after storage under defined conditions.

Exemplary methods to determine stability of the Factor XI and/or Factor XIa antibody in the pharmaceutical formulation are described in Example 1 of the present disclosure. Additionally, stability of the protein can be assessed by measuring the binding affinity of the Factor XI and/or Factor XIa antibody to its targets or the biological activity of the Factor XI and/or Factor XIa antibody in certain in vitro assays, such as the aPTT and FXI activity assays described in WO 2016/207858.

The pharmaceutical formulation can be prepared and stored as a liquid formulation. In certain embodiments, the pharmaceutical formulation is a liquid formulation for storage at 2-8° C. (e.g., 4° C.). In certain embodiments, the pharmaceutical formulation is a liquid formulation for storage at 4° C. and protected from light.

Stability studies have found Antibody 1 150 mg/mL concentrate for solution for injection to be compatible with its excipients and primary packaging materials. Antibody 1 150 mg/mL concentrate for injection is suitable for subcutaneous administration with disposable syringes, without dilution or with dilution in a carrier buffer, e.g., 5% dextrose (D5W). Concentrate for injection with commercially available disposable syringes has been demonstrated for a dose range from 0.5 mg/subject to 600 mg/subject. Materials found to be compatible with Antibody 1 comprise injection syringes composed of polypropylene or polycarbonate, and needles for injection composed of stainless steel. Compatibility of Antibody 1 concentrate for solution for injection has been demonstrated with 1 mL syringes for Antibody 1 concentrations from 0.5 mg/mL to 150 mg/mL. Compatibility of Antibody 1 concentrate for solution for injection has been demonstrated with 3 mL syringes filled up to approximately 2 mL for an Antibody 1 concentration of 150 mg/mL, covering in total a dose range from 0.5 mg up to 150 mg for the 1 mL syringe and a dose of about 300 mg for the 3 mL syringe (filled with approximately 2 mL) per injection.

### Dosage Forms

Prior to pharmaceutical use, the pharmaceutical formulation can be diluted in an aqueous carrier if suitable for the route of administration. For intravenous administration, suitable carriers include sterile water for injection (SWFI), bacteriostatic water for injection (BWFI), a pH buffered solution (e.g., phosphate-buffered saline), sterile saline solution, Ringer's solution, or dextrose solution. For example, when the pharmaceutical formulation is prepared for intravenous administration, the pharmaceutical formulation comprises a 5% dextrose solution (D5W). In certain embodiments, the diluted pharmaceutical formulation is isotonic and suitable for administration by intravenous infusion, e.g., D5W. In certain embodiments, the formulation is diluted in about 50 mL D5W, 100 mL D5W, 150 mL D5W, 200 mL D5W, 250 mL D5W, 300 mL D5W, 350 mL D5W, 400 mL D5W, 450 mL D5W, 500 mL D5W, or 1 L D5W.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 100-200 mg/mL, 100-190 mg/mL, 100-180 mg/mL, 100-170 mg/mL, 100-160 mg/mL, 110-150 mg/mL, 120-150 mg/mL, 130-150 mg/mL, 140-150 mg/mL, 140-160 mg/mL, 140-170 mg/mL, 140-180 mg/mL, 140-190 mg/mL, 150-190 mg/mL, 150-180 mg/mL, 150-170 mg/mL, or 150-160 mg/mL. In certain embodiments, the pharmaceutical formulation comprises the Factor XI and/or Factor XIa antibody at a concentration of about 10 mg/mL, about 15 mg/mL, about 25 mg/mL, about 50 mg/mL, about 75 mg/mL, about 100 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, or about 200 mg/mL.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 1.00-2.00 mg/mL, 1.00-1.90 mg/mL, 1.00-1.80 mg/mL, 1.00-1.70 mg/mL, 1.00-1.60 mg/mL, 1.10-1.50 mg/mL, 1.20-1.50 mg/mL, 1.30-1.50 mg/mL, 1.40-1.50 mg/mL, 1.40-1.60 mg/mL, 1.40-1.70 mg/mL, 1.40-1.80 mg/mL, 1.40-1.90 mg/mL, 1.50-1.90 mg/mL, 1.50-1.80 mg/mL, 1.50-1.70 mg/mL, or 1.50-1.60 mg/mL. In certain embodiments, the pharmaceutical formulation comprises the Factor XI and/or Factor XIa antibody at a concentration of about 0.10 mg/mL, about 0.15 mg/mL, about 0.25 mg/mL, about 0.50 mg/mL, about 0.75 mg/mL, about 1.00 mg/mL, about 1.20 mg/mL, about 1.25 mg/mL, about 1.30 mg/mL, about 1.35 mg/mL, about 1.40 mg/mL, about 1.45 mg/mL, about 1.50 mg/mL, about 1.55 mg/mL, about 1.60 mg/mL, about 1.65 mg/mL, about 1.70 mg/mL, about 1.75 mg/mL, about 1.80 mg/mL, about 1.85 mg/mL, about 1.90 mg/mL, about 1.95 mg/mL, or about 2.00 mg/mL.

In certain embodiments, the pharmaceutical formulation is packaged in a vial (e.g., a vial, bag, pen, or syringe). In certain embodiments, the vial comprises an overfill to allow for complete removal of the intended dose. In certain embodiments, the vial comprises an overfill of 5 to 35%, 10 to 30%, 15 to 25%, or 10 to 20%. In a particular embodiment, the vial comprises an overfill of about 20%.

In certain embodiments, the formulation may be a liquid formulation. In certain embodiments, the amount of Factor XI and/or Factor XIa antibody in the container is suitable for administration as a single dose. In certain embodiments, the amount of Factor XI and/or Factor XIa antibody in the container is suitable for administration in multiple doses. In certain embodiments, the pharmaceutical formulation comprises the Factor XI and/or Factor XIa antibody at an amount of 0.1 to 200 mg. In certain embodiments, the pharmaceutical formulation comprises the Factor XI and/or Factor XIa antibody at an amount of 1 to 200 mg, 10 to 200 mg, 20 to 200 mg, 50 to 200 mg, 100 to 200 mg, 200 to 200 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation comprises the Factor XI and/or Factor XIa antibody at an amount of about 0.1 mg, about 0.5 mg, about 1 mg, about 1.5 mg, about 2 mg, about 2.5 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 450 mg, about 500 mg, about 600 mg, about 700 mg, about 800 mg, about 900 mg, about 1000 mg, about 1500 mg, or about 2000 mg in the therapeutically effective amount.

### Dosage Regimens and Therapeutic Uses

In another aspect, the present disclosure provides a method for treating thromboembolic disease, the method comprising administering to a subject in need thereof a Factor XI and/or Factor XIa antibody disclosed herein (e.g., Antibody 1) once a month.

In certain embodiments, the method further comprises administering to the subject, after the initial treatment cycle, the Factor XI and/or Factor XIa antibody in one or more monthly treatment cycles, e.g., for a period of 3-months, wherein the Factor XI and/or Factor XIa antibody is administered on Day 1, Day 31, and Day 61. The subsequent treatment cycles, in which the subject receives administration of the Factor XI and/or Factor XIa antibody once month, are designed to maintain a certain level of the Factor XI and/or Factor XIa antibody in the subject. In certain embodiments, the subject receives at least 1, 2, 3, 4, 5, 6, 7, 8, 9, 10, 11, 12, 13, 14, or 15 subsequent treatment cycles. In some embodiments, the subject remains on the treatment for life.

In some embodiments, the method comprises treating a disease or disorder in a subject in need thereof, e.g., a thromboembolic disease, comprising administering a first dose of an anti-FXI/FXIa antibody or antigen-binding fragment thereof (e.g., Antibody 1), wherein the first dose is administered intravenously, and administering a second dose of an anti-FXI/FXIa antibody or antigen-binding fragment thereof (e.g., Antibody 1), wherein the second dose is administered subcutaneously. In some embodiments, the method further comprises administering a third dose subcutaneously. In some embodiments, the method further comprises administering a fourth dose subcutaneously. In some embodiments, the method further comprises administering a fifth dose subcutaneously. In some embodiments, the method further comprises administering a sixth dose subcutaneously. In some embodiments, the method further comprises administering a seventh dose subcutaneously. In some embodiments, the method further comprises administering an eighth dose subcutaneously. In some embodiments, the method further comprises administering a ninth dose subcutaneously. In some embodiments, the method further comprises administering a tenth dose subcutaneously. In some embodiments, the method further comprises administering an eleventh dose subcutaneously. In certain embodiments, the method comprises administering a first dose intravenously, and five subsequent doses subcutaneously. In certain embodiments, the treatment duration is about six months. In certain embodiments, the method comprises administering a first dose intravenously, and eleven subsequent doses subcutaneously. In certain embodiments, the treatment duration is about a year. In certain embodiments, the method comprises administering a first dose intravenously, and subsequently administering monthly subcutaneous doses, until resolution of the disease or disorder in the subject, or for the subject's lifetime.

In some embodiments, the subject afflicted with or at risk of developing a thromboembolic disorder and who is undergoing a surgical procedure is administered the intravenous drug delivery formulation on the same day as the surgical procedure. In some embodiments, the intravenous drug delivery formulation is administered between 2 to 10 hours after surgery. In some embodiments, the intravenous drug delivery formulation is administered between 4 to 8 hours after surgery. In some embodiments, the intravenous drug delivery formulation is administered about 1 hour, about 2 hours, about 3 hours, about 4 hours, about 5 hours, about 6 hours, about 7 hours, about 8 hours, about 9 hours, or about 10 hours after surgery.

In certain embodiments, the one or more doses in the initial and subsequent treatment cycles comprise the Factor XI and/or Factor XIa antibody administered subcutaneously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered subcutaneously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, or about 200 mg. In some embodiments, the Factor XI and/or Factor XIa antibody is administered subcutaneously at a dose of about 90 mg. In some embodiments, the Factor XI and/or Factor XIa antibody is administered subcutaneously at a dose of about 120 mg. In some embodiments, the Factor XI and/or Factor XIa antibody is administered subcutaneously at a dose of about 150 mg. In some embodiments, the Factor XI and/or Factor XIa antibody is administered subcutaneously at a dose of about 180 mg. In any of the above embodiments, the Factor XI and/or Factor XIa antibody is administered subcutaneously monthly.

In some embodiments, the therapeutically effective dose range for the Factor XI and/or Factor XIa antibody (e.g., Antibody 1) following subcutaneous administration is about 75 mg to about 165 mg, about 80 mg to about 160 mg, about 85 mg to about 155 mg, or about 90 mg to about 160 mg. In certain embodiments, the therapeutically effective dose range for the Factor XI and/or Factor XIa antibody (e.g., Antibody 1) following subcutaneous administration is about 90 mg to about 160 mg.

In certain embodiments, the one or more doses in the initial and subsequent treatment cycles comprise the Factor XI and/or Factor XIa antibody (e.g., Antibody 1) are administered intravenously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered intravenously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, about 200 mg, about 250 mg, about 500 mg, about 750 mg, about 1000 mg, about 1250 mg, about 1500 mg, or about 2000 mg. In some embodiments, the Factor XI and/or Factor XIa antibody is administered intravenously at a dose of about 30 mg. In some embodiments, the Factor XI and/or Factor XIa antibody is administered intravenously at a dose of about 60 mg. In some embodiments, the Factor XI and/or Factor XIa antibody is administered intravenously at a dose of about 75 mg. In some embodiments, the Factor XI and/or Factor XIa antibody is administered intravenously at a dose of about 150 mg. In some embodiments, the Factor XI and/or Factor XIa antibody is administered intravenously in a single dose. In some embodiments, the FXI/FXIa antibody (e.g., Antibody 1) is administered in a single dose of about 150 mg. In some embodiments, the FXI/FXIa antibody (e.g., Antibody 1) is administered in a single dose of about 1000 mg. In some embodiments, the FXI/FXIa antibody (e.g., Antibody 1) is administered in a single dose of about 1500 mg. In some embodiments, the FXI/FXIa antibody (e.g., Antibody 1) is administered in a single dose of about 2000 mg.

In some embodiments, a first dose of a FXI/FXIa antibody (e.g., Antibody 1) is administered at one dose, and a second dose of the FXI/FXIa antibody (e.g., Antibody 1) is administered at a second dose. In certain embodiments, the first and second dose are the same (e.g., 150 mg). In certain embodiments, the first dose is higher than the second dose (e.g., the first dose is about 1000 mg, and the second dose is 150 mg). In certain embodiments, the method further comprises administration of subsequent doses at the same dosage as the second dose.

In certain embodiments, administering a single dose of a FXI/FXIa antibody (e.g., Antibody 1) prolongs activated partial thromboplastin time (aPTT) by 400 hours or more, e.g., 400 hours or more, 500 hours or more, or 600 hours or more. In certain embodiments, administering a single dose of a FXI/FXIa antibody (e.g., Antibody 1) prolongs activated partial thromboplastin time (aPTT) by about 600 hours.

A physician can start doses of the antibodies of the present disclosure (e.g., Antibody 1) employed in the pharmaceutical composition at levels lower than that required to achieve the desired therapeutic effect and gradually increase the dosage until the desired effect is achieved. In general, effective doses of the compositions of the present disclosure, for the treatment of thromboembolic disorders described herein vary depending upon many different factors, including means of administration, target site, physiological state of the patient, other medications administered, and whether treatment is prophylactic or therapeutic. Treatment dosages may be titrated to optimize safety and efficacy. For systemic administration with an antibody, the dosage ranges from about 0.01 to 15 mg/kg of the host body weight. For administration (e.g., subcutaneous or intravenous administration) with an antibody, the dosage may range from 0.1 mg to 5 mg or from 1 mg to 600 mg. For example, an anti-FXI/FXIa antibody described herein (e.g., Antibody 1) can be administered at a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the Factor XI and/or Factor XIa antibody is administered intravenously. For example, in certain embodiments, the Factor XI and/or Factor XIa antibody is administered by intravenous infusion, e.g., with a prefilled bag, a prefilled pen, or a prefilled syringe. In certain embodiments, the Factor XI and/or Factor XIa antibody, in a pharmaceutical formulation disclosed herein, is diluted prior to administration. For example, in certain embodiments, the pharmaceutical formulation is diluted with dextrose 5% in water (D5W) and is administered intravenously from a bag. The intravenous infusion may be for about one hour (e.g., 50 to 80 minutes). In certain embodiments, the bag is connected to a channel comprising a tube and/or a needle.

In certain embodiments, the Factor XI and/or Factor XIa antibody is administered parenterally. In certain embodiments, the Factor XI and/or Factor XIa antibody is administered parenterally in one or more doses.

The types of thromboembolic disorders that can be treated with the Factor XI and/or Factor XIa antibody or pharmaceutical formulation disclosed herein include but are not limited to a “thromboembolic,” or similar terms as used herein, can also refer to any number of the following, which the anti-FXI and/or FXIa antibodies or antigen binding fragments thereof of the present disclosure can be used to prevent or treat: thromboembolism in subjects with suspected or confirmed cardiac arrhythmia such as paroxysmal, persistent or permanent atrial fibrillation or atrial flutter; stroke prevention in atrial fibrillation (SPAF), a subpopulation of which is AF patients undergoing percutaneous coronary interventions (PCI); acute venous thromboembolic events (VTE) treatment and extended secondary VTE prevention in patients at high risk for bleeding; cerebral and cardiovascular events in secondary prevention after transient ischemic attack (TIA) or non-disabling stroke and prevention of thromboembolic events in heart failure with sinus rhythm; venous thromboembolism in pediatric subjects (pediatric VTE); clot formation in left atrium and thromboembolism in subjects undergoing cardioversion for cardiac arrhythmia; thrombosis before, during and after ablation procedure for cardiac arrhythmia; venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins; thrombosis on any artificial surface in the veins or arteries like catheter, pacemaker wires, synthetic arterial grafts; arterio-venous (AV) shunt; mechanical or biological heart valves or left ventricular assist device; pulmonary embolism in patients with or without venous thrombosis; Chronic Thromboembolic Pulmonary Hypertension (CTEPH); arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension; thrombosis and thromboembolism in patients undergoing percutaneous coronary interventions (PCI); cardioembolic and cryptogenic strokes; non-central nervous systemic embolism (non-CNS systemic embolism); hemorrhagic stroke; thrombosis in patients with invasive and non-invasive cancer malignancies (e.g., gastrointestinal cancer and genitourinary cancer); thrombosis over an indwelling catheter; thrombosis and thromboembolism in severely ill patients; cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart; thromboembolism in patients with valvular heart disease with or without atrial fibrillation; thromboembolism over valvular mechanic or biologic prostheses; thromboembolism in patients who had native or artificial cardiac patches, arterial or venous conduit tubes after heart repair of simple or complex cardiac malformations; venous thrombosis and thromboembolism after knee replacement surgery, hip replacement surgery, and orthopedic surgery, thoracic or abdominal surgery; arterial or venous thrombosis after neurosurgery including intracranial and spinal cord interventions; congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia; thrombosis and thromboembolism in chronic kidney disease; and thrombosis and thromboembolism in patients undergoing hemodialysis and in patients undergoing extra-corporal membrane oxygenation. In certain embodiments, the subject treated with the Factor XI and/or Factor XIa antibody or pharmaceutical formulation disclosed herein is obese (e.g., severely obese, e.g., with body-mass index (BMI)≥35 kg/m²). In certain embodiments, the subject treated with the Factor XI and/or Factor XIa antibody or pharmaceutical formulation disclosed herein is not obese. In certain embodiments, the obese subject is associated with lower exposure following administration of the same dose of the Factor XI and/or Factor XIa antibody (e.g., Antibody 1), as the non-obese subject. In certain embodiments, the exposure is about 10%, about 20%, about 30%, about 40%, about 50%, about 60%, about 70%, about 80%, or about 90% less for the obese subject following administration of the same dose of the Factor XI and/or Factor XIa antibody (e.g., Antibody 1), as the non-obese subject. In certain embodiments, the obese subject is associated with shorter duration of aPTT prolongation following administration of the same dose of the Factor XI and/or Factor XIa antibody (e.g., Antibody 1), as the non-obese subject. In certain embodiments, the aPTT prolongation is about 10%, about 20%, about 30%, about 40%, about 50%, about 60%, about 70%, about 80%, or about 90% shorter for the obese subject following administration of the same dose of the Factor XI and/or Factor XIa antibody (e.g., Antibody 1), as the non-obese subject.

In certain embodiments, the anti-FXI/FXIa antibody or antigen-binding fragment described herein (e.g., Antibody 1) is administered to a patient having thrombocytopenia. “Thrombocytopenia” refers to a condition in which a subject has lower-than-normal blood platelet levels, e.g., lower than physiologically average blood platelet levels in a particular population. In some embodiments, a patient having a thrombocytopenia has a platelet count below 150×10⁹/L, or about the 2.5th percentile of the normal platelet count distribution. Patients having thrombocytopenia have increased bleeding risk but can still be at risk for thromboembolic disorders (e.g., venous thromboembolism (VTE) or stroke). Treating patients having thrombocytopenia with existing anticoagulant therapies can be clinically challenging due to the need to balance increased bleeding risk with the need to treat, prevent, or reduce the risk of thromboembolic disorders. In certain embodiments, administration of the anti-FXI/FXIa antibody or antigen-binding fragment described herein (e.g., Antibody 1) to a patient having thrombocytopenia has a lower risk of bleeding compared to administration of another anticoagulant therapy. In some embodiments, administering of an antibody or antigen-binding fragment described herein (e.g., Antibody 1) does not affect platelet aggregation in the subject as compared to platelet aggregation prior to the administering. Platelet aggregation may be determined, for example and without limitation, by impedance platelet aggregometry, and other suitable methods as known in the art.

In some embodiments, the thrombocytopenia is associated with cirrhosis. Cirrhosis can decrease both procoagulant and anticoagulant factor production, increasing risk of bleeding and/or risk of thrombosis in patients afflicted with cirrhosis. Factor XI inhibition (e.g., by Antibody 1) may prevent development of portal vein thrombosis and reduce hepatic decompensation events, as was previously demonstrated using a low molecular weight heparin (LMWH), enoxaparin (Villa et al. (2012). Gastroenterology. 143(5):1253-1260). Use of LMWH for cirrhosis is, however, limited by user compliance (e.g., daily administration) and risk, as advanced cirrhosis is often complicated by renal failure, where administration of heparin is not recommended. In some embodiments, administration of the anti-FXI/FXIa antibody or antigen-binding fragment described herein (e.g., Antibody 1) to a patient having cirrhosis slows progression of the cirrhosis. In some embodiments, administration of the anti-FXI/FXIa antibody or antigen-binding fragment described herein (e.g., Antibody 1) to a patient having cirrhosis prevents or reduces the incidence of intrahepatic microthrombi. In some embodiments, administration of the anti-FXI/FXIa antibody or antigen-binding fragment described herein (e.g., Antibody 1) to a patient having cirrhosis decreases the incidence of hepatic ischemia and/or fibrosis. In some embodiments, administration of the anti-FXI/FXIa antibody or antigen-binding fragment described herein (e.g., Antibody 1) to a patient having cirrhosis prevents or reduces the risk of development of portal vein thrombosis. In some embodiments, administration of the anti-FXI/FXIa antibody or antigen-binding fragment described herein (e.g., Antibody 1) to a patient having cirrhosis decreases the incidence of cirrhotic sequelae (e.g., esophageal varices and variceal bleeding), e.g., as compared to the incidence of cirrhotic sequelae in the patient prior to administration. In some embodiments, administration of the anti-FXI/FXIa antibody or antigen-binding fragment described herein (e.g., Antibody 1) to a patient having cirrhosis reduces the number of hepatic decompensation events, e.g., as compared to the number of hepatic decompensation events of the patient prior to administration.

In some embodiments, the thrombocytopenia is associated with an infection. In certain embodiments, the thrombocytopenia is associated with a human immunodeficiency virus (HIV) or hepatitis C virus. In some embodiments, the thrombocytopenia is congenital. In certain embodiments, the congenital thrombocytopenia is Wiskott-Aldrich syndrome, thrombocytopenia-absent radii syndrome, Bernard-Soulier, Gray platelet syndrome, familial-thrombocytopenia-leukemia syndrome, MYH9-related disorders, Fanconi Anemia, congenital amegakaryocytic thrombocytopenia, or dyskeratosis congenita. In some embodiments, the thrombocytopenia is idiopathic. In some embodiments, the idiopathic thrombocytopenia is idiopathic thrombocytopenia purpura (ITP).

In certain embodiments, the Factor XI and/or Factor XIa antibody (e.g., Antibody 1), is administered to a subject with a cancer. It is estimated that venous thromboembolism (VTE) affects between 1% and 8% of all cancer patients, and further, is the second leading cause of death in outpatients undergoing chemotherapy. Treatment of such VTE in cancer patients is complicated by thrombocytopenia, which is widely prevalent in cancer patients undergoing chemotherapy (Bannow et al. (2019). Res. Pract. Throm. Haemost., 2:664-669). In some embodiments, the subject, e.g., a cancer subject, has thrombocytopenia. As used herein, “cancer subject” or “subject with a cancer” is a subject, e.g., a human subject, diagnosed as having a cancer. In certain embodiments, the cancer subject is undergoing treatment, e.g., chemotherapy, for said cancer. In some embodiments, the thrombocytopenia is associated with chemotherapy. In some embodiments, the thrombocytopenia is induced by chemotherapy.

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the cancer is selected from the group consisting of gastrointestinal cancer and genitourinary cancer.

In some embodiments, the anti-FXI/FXIa antibody or antigen-binding fragment described herein (e.g., Antibody 1) is administered preventively, e.g., to prevent a clotting in a subject at risk of thrombosis. In some embodiments, the anti-FXI/FXIa antibody or antigen-binding fragment described herein (e.g., Antibody 1) is administered therapeutically, e.g., to treat a clot in a subject at risk of thrombosis.

In some embodiments, the anti-FXI/FXIa antibody or antigen-binding fragment thereof (e.g., Antibody 1) is administered to a subject with a cancer-associated thrombosis (CAT). In certain embodiments, the subject with the cancer-associated thrombosis has an existing clot. In certain embodiments, the subject has a microclot. In certain embodiments, the subject has microthombosis associated with CAT.

The CHA2DS2-VASc risk score is a validated and widely used stratification tool to predict thromboembolic risk in AF patients and to identify patients who should benefit from anticoagulation therapy (LIP 2011; Camm, et al. (2012) Eur Heart J 2012; 33: 2719-2747); the accumulated evidence shows that CHA2DS2-VASc is at least as accurate as or possibly better than, scores such as CHADS2 in identifying patients who develop stroke and thromboembolism and definitively better at identifying ‘truly low-risk’ patients with AF. The CHA2DS2-VASc risk score ranges from 0 to a maximum score of 9. In certain embodiments, the subject treated with the Factor XI and/or Factor XIa antibody or pharmaceutical formulation disclosed herein has a CHA2DS2-VASc risk score of 0-1 for men and 1-2 for women. In certain embodiments, the subject treated with the Factor XI and/or Factor XIa antibody or pharmaceutical formulation disclosed herein has a CHA2DS2-VASc risk score≥2 for men and ≥3 for women. In certain embodiments, the subject treated with the Factor XI and/or Factor XIa antibody or pharmaceutical formulation disclosed herein has a CHA2DS2-VASc risk score≥4 or ≥3 with at least 1 of planned concomitant use of anti-platelet medication (e.g., aspirin and/or P2Y12 inhibitor) or CrCl≤50 ml/min by the Cockcroft-Gault equation.

The Factor XI and/or Factor XIa antibody disclosed herein (e.g., Antibody 1) can be used as a monotherapy or in combination with one or more therapies. Such combination therapies may be useful for treating thromboembolic disorders, such as, ischemic stroke (cardioembolic, thrombotic) or systemic embolism, AF, stroke prevention in AF (SPAF), deep vein thrombosis, venous thromboembolism, pulmonary embolism, acute coronary syndromes (ACS), acute limb ischemia, chronic thromboembolic pulmonary hypertension, or systemic embolism). In certain embodiments, the Factor XI and/or Factor XIa antibody is used as a monotherapy in accordance with the dosage regimen disclosed herein. In other embodiments, the Factor XI and/or Factor XIa antibody is used in combination with one or more therapies, wherein the Factor XI and/or Factor XIa antibody is administered in accordance with the dosage regimen disclosed herein and the one or more therapies are administered in accordance with a dosage regimen known to be suitable for treating the particular subject with the particular disorder.

In some aspects, statin therapies may be used in combination with the FXI/FXIa antibodies and antigen binding fragments, or formulations comprising said FXI/FXIa antibodies and antigen binding fragments (e.g., Antibody 1), described in the present disclosure for the treatment of patients with thrombotic and/or thromboembolic disorders. In particular aspects, non-limiting examples of therapeutic active agents suitable for use in combination with an anti-FXI/FXIa antibody described herein (e.g., Antibody 1) include thromboxane inhibitors (e.g., aspirin), adenosine diphosphate receptor antagonists (or P2Y12 inhibitors) such as thienopyridines (e.g., clopidogrel and prasugrel) and nonthienopyridines (e.g., ticagrelor and cangrelor), protease-activated receptor-1 (PAR1) antagonists (e.g., vorapaxar and atopaxar), and proton pump inhibitors (PPIs) (e.g., omeprazole, diazepam, phenytoin, lansoprazole, dexlansoprazole, rabeprazole, pantoprazole, esomeprazole, and naproxen). The use of PPIs in combination therapy may be suitable in cases where a subject has or has a history of a GI disorder, such as previous GI bleed or antecedent of peptic ulcer. In one aspect, the subject is being treated with non-steroidal anti-inflammatory drugs (NSAIDs), and is administered an anti-FXI/FXIa antibody described herein (e.g., Antibody 1) in combination with a proton pump inhibitor (e.g., omeprazole, diazepam, phenytoin, lansoprazole, dexlansoprazole, rabeprazole, pantoprazole, esomeprazole, and naproxen). In certain embodiments, a subject treated with the FXI/FXIa antibodies and antigen binding fragments, or formulations comprising said FXI/FXIa antibodies and antigen binding fragments (e.g., Antibody 1), are administered a direct oral anticoagulant (DOAC) following the duration of treatment (e.g., on the same day as end of treatment). In certain embodiments, a subject treated with the FXI/FXIa antibodies and antigen binding fragments, or formulations comprising said FXI/FXIa antibodies and antigen binding fragments (e.g., Antibody 1), are administered a Vitamin K Antagonist (VKA) following the duration of treatment (e.g., about 5 days before end of treatment, or about 3 days before end of treatment).

In certain embodiments, the method of treatment disclosed herein results in a disease response or improved survival of the subject or patient. For example, in certain embodiments, the disease response is a complete response, a partial response, or a stable disease. In certain embodiments, the improved survival is improved progression-free survival (PFS) or overall survival. Improvement (e.g., in PFS) can be determined relative to a period prior to initiation of the treatment of the present disclosure. Methods of determining disease response (e.g., complete response, partial response, or stable disease) and patient survival (e.g., PFS, overall survival) for BTC (e.g., advanced BTC, metastatic BTC), or biliary tract tumor therapy, are routine in the art and are contemplated herein. In some embodiments, disease response is evaluated according to RECIST 1.1 after subjecting the treated patient to contrast-enhanced computed tomography (CT) or magnetic resonance imaging (MRI) of the affected area (e.g., chest/abdomen and pelvis covering the area from the superior extent of the thoracic inlet to the symphysis pubis).

### ENUMERATED EMBODIMENTS

1. A method of treating a disease or disorder in a subject in need thereof, the method comprising intravenously administering to the subject a first dose of about 150 mg of an isolated anti-Factor XI (FXI) and/or anti-activated Factor XI (FXIa) antibody, or an antigen-binding fragment thereof, and subcutaneously administering to the subject a second dose of the isolated anti-FXI and/or anti-FXIa antibody, or the antigen-binding fragment thereof.

2. The method of embodiment 1, wherein the second dose comprises about 150 mg of the isolated anti-FXI and/or anti-FXIa antibody, or the antigen-binding fragment thereof.

3. The method of embodiment 1 or 2, wherein the first dose of the isolated anti-FXI and/or anti-FXIa antibody, or the antigen-binding fragment thereof, is formulated as an intravenous drug delivery formulation comprising about 150 mg of the antibody, or the antigen-binding fragment thereof.

4. The method of any one of embodiments 1-3, wherein the second dose of the isolated anti-FXI and/or anti-FXIa antibody, or the antigen-binding fragment thereof, is formulated as a subcutaneous drug delivery formulation comprising about 150 mg of the antibody or the antigen-binding fragment thereof.

5. The method of any one of embodiments 1-4, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

6. The method of any one of embodiments 1-5, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

7 The method of any one of embodiments 1-6, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

8 The method of any one of embodiments 1-7, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

9. The method of any one of embodiments 1-8, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

10. The method of any one of embodiments 1-9, wherein the antibody comprises a heavy chain comprising an amino acid sequence of SEQ ID NO: 31 and a light chain comprising an amino acid sequence of SEQ ID NO: 41.

11. The method of any one of embodiments 1-10, wherein the antibody is a human monoclonal antibody.

12. The method of embodiment 11, wherein the antibody is a human IgG1 isotype.

13. The method of embodiment 11 or 12, wherein the antibody comprises D265A and P329A substitutions in the Fc domain.

14. The method of any one of embodiments 1-13, wherein the antibody or antigen-binding fragment thereof is administered in a drug delivery formulation comprising a histidine buffer at a concentration of about 20 mM.

15. The method of any one of embodiments 1-14, wherein the antibody or antigen-binding fragment thereof is administered in a drug delivery formulation comprising sucrose at a concentration of about 220 mM.

16. The method of any one of embodiments 1-15, wherein the antibody or antigen-binding fragment thereof is administered in a drug delivery formulation comprising polysorbate 20 at a concentration of about 0.04%.

17. The method of any one of embodiments 1-16, wherein the antibody or antigen-binding fragment thereof is administered in a drug delivery formulation at pH 5.5.

18. The method of any one of embodiments 1-17, wherein, when the antibody or antigen-binding fragment thereof is administered in an intravenous drug delivery formulation, the intravenous drug delivery formulation further comprises about 5% glucose.

19. The method of any one of embodiments 1-18, wherein the subject has a cancer.

20. The method of any one of embodiments 1-19, wherein the subject has a cancer selected from the group consisting of gastrointestinal cancer and genitourinary cancer.

21. The method of any one of embodiments 1-20, wherein the subject is at high risk of venous thromboembolism.

22. The method of any one of embodiments 1-21, wherein the subject has had one or more previous venous thromboembolisms.

23. The method of any one embodiments 1-22, wherein the method further comprises one or more additional subcutaneous doses of the antibody or antigen-binding fragment thereof.

24. The method of any one of embodiments 1-23, wherein the method comprises administering five subcutaneous doses of the antibody or antigen-binding fragment thereof.

25. The method of any one of embodiments 1-24, wherein the antibody or antigen-binding fragment thereof is administered subcutaneously about once a month.

26. The method of any one of embodiments 1-25, wherein the antibody or antigen-binding fragment thereof is administered intravenously on day 1 and is administered subcutaneously on days 31, 61, 91, 121, and 151.

27. The method of any one of embodiments 1-26, wherein the subject is treated for about six months.

28. A method of treating a subject with a cancer, wherein the method comprises administering a drug delivery formulation comprising about 150 mg of an isolated anti-Factor XI (FXI) and/or anti-activated Factor XI (FXIa) antibody or antigen-binding fragment thereof to the subject in need thereof, wherein the drug delivery formulation is administered once intravenously and subsequently is administered subcutaneously about once a month, and wherein the subject is treated for about six months.

29. The method of embodiment 28, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

30. The method of any one of embodiments 28-29, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

31. The method of any one of embodiments 28-30, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

32. The method of any one of embodiments 28-31, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

33. The method of any one of embodiments 28-32, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

34. The method of any one of embodiments 28-33, wherein the antibody comprises a heavy chain comprising an amino acid sequence of SEQ ID NO: 31 and a light chain comprising an amino acid sequence of SEQ ID NO: 41.

35. The method of any one of embodiments 28-34, wherein the antibody is a human monoclonal antibody.

36. The method of embodiment 35, wherein the antibody is a human IgG1 isotype.

37. The method of embodiment 35 or 36, wherein the antibody comprises D265A and P329A substitutions in the Fc domain.

38. The method of any one of embodiments 28-37, wherein the cancer is selected from the group consisting of gastrointestinal cancer and genitourinary cancer.

39. A method of treating a primate subject at risk of thrombosis, wherein the method comprises administering to the primate subject a single dose of a drug delivery formulation comprising:
(a) a therapeutically effective amount of an isolated anti-Factor XI (FXI) and/or anti-activated Factor XI (FXIa) antibody, or antigen-binding fragment thereof at a concentration of about 150 mg;
(b) a histidine buffer at a concentration of about 20 mM;
(c) sucrose at a concentration of about 220 mM; and
(d) polysorbate-20 at a concentration of about 0.04% (v/v),
(e) a diluent comprising glucose,
at pH 5.5,
and wherein the administering is before or during formation of a blood clot.

40. The method of embodiment 39, wherein the primate subject is a baboon.

41. The method of embodiment 39, wherein the primate subject is a human.

42. The method of embodiment 39 or 40, wherein the thrombosis is an experimentally-induced thrombosis.

43. The method of any one of embodiments 39-42, wherein the primate subject is at risk of vascular graft thrombosis.

44. The method of any one of embodiments 39-43, wherein the single dose is administered to prevent thrombosis.

45. The method of any one of embodiments 39-43, wherein the single dose is administered to treat thrombosis.

46. The method of any one of embodiments 39-45, wherein the single dose is parenteral or intravenous.

47. The method of any one of embodiments 39-46, wherein the single dose is parenteral.

48. The method of any one of embodiments 39-47, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

49. The method of any one of embodiments 39-48, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

50. The method of any one of embodiments 39-49, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

51. The method of any one of embodiments 39-50, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

52. The method of any one of embodiments 39-51, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

53. The method of any one of embodiments 39-52, wherein the antibody comprises a heavy chain comprising an amino acid sequence of SEQ ID NO: 31 and a light chain comprising an amino acid sequence of SEQ ID NO: 41.

54. The method of any one of embodiments 39-53, wherein the antibody is a human monoclonal antibody.

55. The method of embodiment 54, wherein the antibody is a human IgG1 isotype.

56. The method of embodiment 54 or 55, wherein the antibody comprises D265A and P329A substitutions in the Fc domain.

57. The method of any one of embodiments 39-40 or 42-56, wherein about 1 mg/kg is the therapeutically effective amount of the anti-Factor XI (FXI) and/or anti-activated Factor XI (FXIa) antibody or antigen-binding fragment thereof, for administration to the primate subject.

58. The method of any one of embodiments 39, 41, or 43-56, wherein about 150 mg is the therapeutically effective amount of the anti-Factor XI (FXI) and/or anti-activated Factor XI (FXIa) antibody or antigen-binding fragment thereof, for administration to the primate subject.

59. A method of treating a subject having a thrombocytopenia, wherein the thrombocytopenia is selected from the group consisting of: chemotherapy-induced thrombocytopenia, congenital thrombocytopenia, thrombocytopenia associated with infection, and idiopathic thrombocytopenia, the method comprising administering a therapeutically effective amount of a Factor XI and/or Factor XIa antibody, or an antigen-binding fragment thereof, to the subject in need thereof.

60. The method of embodiment 59, wherein the subject having a thrombocytopenia has a cancer.

61. The method of embodiment 59 or 60, wherein the subject having a thrombocytopenia has cirrhosis.

62. The method of embodiment 59 or 60, wherein the subject having a thrombocytopenia has idiopathic thrombocytopenia purpura (ITP).

63. A method of treating a cancer subject having a chemotherapy-induced thrombocytopenia, wherein the method comprises administering a therapeutically effective amount of a Factor XI and/or Factor XIa antibody, or an antigen-binding fragment thereof, to the cancer subject in need thereof.

64. The method of any one of embodiments 59-63, wherein the subject or the cancer subject is afflicted with or at risk of developing a thromboembolic disorder.

65. The method of any one of embodiments 59-64, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

66. The method of any one of embodiments 59-65, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

67. The method of any one of embodiments 59-66, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

68. The method of any one of embodiments 59-67, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

69. The method of any one of embodiments 59-68, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

70. The method of any one of embodiments 59-69, wherein the antibody comprises a heavy chain comprising an amino acid sequence of SEQ ID NO: 31 and a light chain comprising an amino acid sequence of SEQ ID NO: 41.

71. The method of any one of embodiments 59-70, wherein the antibody is a human monoclonal antibody.

72. The method of embodiment 71, wherein the antibody is a human IgG1 isotype.

73. The method of embodiment 71 or 72, wherein the antibody comprises D265A and P329A substitutions in the Fc domain.

74. The method of any one of embodiments 59-73, wherein the administering of the antibody or antigen-binding fragment thereof does not affect platelet aggregation in the subject as compared to platelet aggregation prior to the administering.

75. The method of embodiment 74, wherein the platelet aggregation is measured by impedance platelet aggregometry.

76. The method of embodiment 75, wherein the platelet aggregation is induced by collagen, adenosine 5′-diphosphate (ADP), or thrombin receptor activating peptide-6 (TRAP-6).

77. The method of any one of embodiments 74-76, wherein the platelet aggregation is determined ex vivo or in vitro.

78. The method of any one of embodiments 59-77, wherein the antibody or antigen-binding fragment thereof is administered intravenously.

79. The method of any one of embodiments 59-78, wherein the antibody or antigen-binding fragment thereof is administered subcutaneously.

80. The method of any one of embodiments 59-78, wherein a first dose of the antibody or antigen-binding fragment thereof is administered intravenously and a second dose of the antibody or antigen-binding fragment is administered subcutaneously.

81. The method of embodiment 80, further comprising one or more additional doses of the antibody or antigen-binding fragment thereof administered subcutaneously following the administering of the second dose.

82. The method of any one of embodiments 59-81, wherein the antibody or antigen-binding fragment thereof is administered once a month.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1—Treatment of Patients with Cancer-Associated Thromboembolism Antibody 1 Compared to Apixaban

### Purpose and Rationale

The purpose of this study is to assess whether monthly treatment with Antibody 1 is non-inferior to twice daily (bid) oral administration of apixaban in preventing venous thromboembolism (VTE) recurrence but is superior in the rate of bleeding in patients with cancer and recently diagnosed VTE. This study will support worldwide registration of Antibody 1 for the treatment of cancer-associated VTE. (Cancer-associated thromboembolism). CAT occurs in an estimated 20% of cancer patients and is the second leading cause of death in patients with malignancies. Current treatments inhibit one or more factors in the coagulation cascade and while they effectively prevent or treat thrombosis, they also interfere with hemostasis resulting in a high risk of bleeding. The fear of bleeding and the lack of tolerability of available therapies leads to significant undertreatment and poor outcomes.

The 2 most common treatments today are low-molecular weight heparin (LMWH) and direct oral anticoagulants (DOACs), and each has limitations. LMWH is the SoC in most parts of the world, but it requires daily injections for 6 months. Unfortunately, patients treated with LMWH are less likely to persist on therapy compared to other orally administered treatment. DOACs are administered orally and are seen as a more tolerable alternative, but some cancer patients have difficulty swallowing or develop vomiting which leads to poor adherence.

The goal of developing a new treatment for CAT, and especially patients with CAT who are at high risk for bleeding, is to maintain the same level of efficacy as current agents but with less bleeding and greater tolerability.

### Objectives and Endpoints

The primary objective of this study is to assess whether Antibody 1 is non-inferior to apixaban for preventing VTE recurrence through 6 months post randomization in patients with cancer and recently diagnosed VTE, with the endpoint being time to first event of centrally adjudicated VTE recurrence through 6 months.

The secondary objectives of this study are:
To assess whether Antibody 1 is superior to apixaban for preventing occurrence of the composite of major or clinically relevant non-major (CRNM) bleeding through 6 months post randomization, with the endpoint being time to first event of International Society on Thrombosis and Haemostasis (ISTH)-adjudicated major or CRNM bleeding events through 6 months.
To assess whether Antibody 1 is superior to apixaban on net clinical benefit defined as survival without VTE recurrence, or major or CRNM bleeding events through 6 months post randomization, with the endpoint being time to first event of VTE recurrence, ISTH-adjudicated major or ISTH-adjudicated CRNM bleeding events through 6 months.
To assess whether Antibody 1 is superior to apixaban for preventing VTE recurrence at 6 months post randomization, with the endpoint being time to first event of centrally adjudicated VTE recurrence through 6 months.
To assess whether Antibody 1 is superior to apixaban on the rate of permanent treatment discontinuation not due to death through 6 months post randomization, with the endpoint being time to event of permanent treatment discontinuation not due to death.
To assess whether Antibody 1 is superior to apixaban for preventing occurrence of CRNM bleeding events through 6 months post randomization, with the endpoint being time to first event of ISTH-adjudicated CRNM bleeding events through 6 months.
To assess whether Antibody 1 is superior to apixaban for preventing occurrence of major bleeding events through 6 months post randomization, with the endpoint being time to first event of ISTH-adjudicated major bleeding events through 6 months.
To assess whether Antibody 1 is superior to apixaban for preventing occurrence of the composite of GI major and GI CRNM bleeding through 6 months post randomization, with the endpoint being time to first event of GI ISTH-adjudicated major and CRNM bleeding events through 6 months.
To evaluate safety and tolerability of Antibody 1 relative to dalteparin through 6 months post randomization and to assess incidence rate of injection site reactions, hypersensitivity reactions and immunogenicity in patients treated with Antibody 1, with the endpoints being all-cause death, vascular death, serious adverse events, adverse events leading to drug discontinuation, other adverse events, abnormal lab tests, etc. presented as rate per 100 patient-years. Additional endpoints for patients treated with Antibody 1 are:
   Percentage of patients with injection site reactions
   Percentage of patients with injection site reactions by severity status
   Percentage of patients with hypersensitivity reactions
   Percentage of patients with hypersensitivity reactions by severity status
   Percentage of patients with ADA formation
   Percentage of patients with persistent ADA formation
   Percentage of patients with neutralizing antibody (NAb) formation.

Exploratory objectives for this study are:
To evaluate the effect of Antibody 1 relative to apixaban on the composite of VTE recurrence, major bleeding events or all-cause death, with the endpoint being time to first event of the composite endpoint of VTE recurrence, major bleeding, or all-cause death.
To assess the effect of Antibody 1 relative to apixaban on arterial thromboembolic events (e.g., stroke, myocardial infarction, arterial embolic events), with the endpoint being time to first event of the composite endpoint of ischemic stroke, or myocardial infarction or arterial embolic events.
To assess the effect of Antibody 1 relative to apixaban on venous thromboembolic events other than events qualifying for VTE recurrence (e.g., leg distal DVT, upper extremity DVT, thoracic veins, intracranial or extracranial cerebral veins, splanchnic DVT and central venous line associated thrombosis), with the endpoints being time to first event of the composite endpoint of DVT recurrence or other venous thromboembolic events and time to the first event of the following venous thromboembolic events: leg distal DVT, upper extremity DVT, thoracic veins, intracranial or extracranial cerebral veins, splanchnic DVT or central venous line associated thrombosis.
To assess the effect of Antibody 1 relative to apixaban on the number and duration of temporary treatment interruptions that are not due to procedures or bleeding events, with the endpoints being number of temporary treatment interruptions that are not due to procedures or bleeding and total duration of temporary treatment interruptions that are not due to procedures or bleeding.
To assess efficacy and safety of Antibody 1 relative to apixaban in predefined subgroups of patients (e.g., gender, age, BMI, ethnicity, cancer location, incidental vs. symptomatic VTE, presence or absence of PE, days on SoC prior to randomization, ECOG), with the endpoints being time to first event of centrally adjudicated VTE recurrence through 6 months in predefined subgroups of patients and time to first event of ISTH-defined major and CRNM bleeding events through 6 months in the predefined subgroups of patients.
To assess the effect of Antibody 1 relative to apixaban on health-related quality of life (HRQOL) at 3 and 6 months and to assess changes over time in EQ-5D-5L in patients who present suspected VTE recurrence and suspected bleeding events, with the endpoints being change from baseline in the overall score and score by domain of
   EQ-5D-5L questionnaire
   EORTC QLQ C30 questionnaire
   TSQM II questionnaire
and serial changes over time in EQ-5D-5L questionnaire following suspected VTE recurrence and bleeding events.
To assess the PK of Antibody 1 in a subset of patients, with the endpoint being random, peak and trough Antibody 1 plasma concentrations.
To assess the PD (free and total FXI, FXI:C, aPTT) of Antibody 1 in a subset of patients, with the endpoint being free and total FXI, FXI:C and aPTT, at indicated time points.
To assess the effect on Antibody 1 relative to apixaban on exploratory biomarkers of hypercoagulable state (potentially including but not limited to d-dimer, F1.2, TAT) in subset of patients, with the endpoint being changes from baselines in biomarkers of hypercoagulable state that may include d-dimer, F1.2 and TAT between treatment arms.
VTE cost effectiveness analysis (e.g., hospital stay, transfusion, procedures to stop bleeding or to manage VTE, physicians' office visits), with the endpoint being healthcare resource utilization
DNA assessments to explore whether individual genetic variation in genes relating to the drug target pathway or other relevant genetic pathways confer differential responses to Antibody 1, with the endpoint being exploratory evaluation of the association between gene polymorphisms and safety, efficacy, PK, and PD response

### Study Design

This is a randomized, open-label, blinded endpoint evaluation (PROBE), active controlled study comparing the effect of Antibody 1 relative to apixaban on VTE recurrence in patients with cancer.

Patients with cancer confirmed by histology and/or adequate imaging modality and a newly diagnosed, objectively confirmed symptomatic or incidental proximal lower-limb acute DVT or symptomatic pulmonary embolism (PE), or incidentally detected PE in a segmental or more proximal pulmonary artery are eligible for study participation within 72 hours of diagnosis of VTE.

After presentation, patients who consent to study participation should undergo a screening/run-in period of up to 72 hours to confirm eligibility criteria. A SOC treatment for VTE (e.g., recommended dose of a direct oral anticoagulant [DOAC], unfractionated heparin [UFH], LMWH or fondaparinux) should be administered during the screening/run-in period. Patients who continue to meet all inclusion and exclusion criteria will be randomized to Antibody 1 or apixaban in a 1:1 ratio. Patients will be stratified by study region, cancer location (GI/GU vs. other), and symptomatic vs incidental VTE. Patients assigned to Antibody 1 will receive Antibody 1 150 mg intravenous (iv) on Day 1, followed by once monthly 150 mg subcutaneous (sc) dosing approximately 30 days (±5 days) after the iv dose for 5 additional months (6 total treatments). Patients assigned to apixaban will directly start 10 mg of oral (po) apixaban twice-daily (bid) for 7 days following randomization, then 5 mg po bid for a total treatment duration of 6 months. If a patient receives apixaban at a dose of 10 mg bid without interruption during screening and is randomized to the apixaban treatment arm, the on-study treatment duration with the 10 mg bid dose will be shortened so no patient receives apixaban 10 mg bid dose for more than 7 days in total.

The index VTE events, suspected recurrent VTE events, death, bleeding events, arterial thromboembolic events, and other venous thromboembolic events will be reviewed by a Clinical Event Committee (CEC) whose members will be blinded to treatment assignment. The above-mentioned events occurring up to the end of study visit will be adjudicated in all randomized patients.

Patients who complete 6 months of treatment or who discontinue the study treatment prematurely will enter a follow-up period of up to 70 days.

### Population

Adult male or female subjects are eligible for study participation if they have confirmed (histology, adequate imaging modality) unresectable GI or GU cancer prior to randomization and presentation of acute VTE (within 72 hours of diagnosis of the qualifying VTE), for which long-term treatment with LMWH is indicated; approximately 1020 patients will be randomized in this study.

### Inclusion Criteria

Inclusion and exclusion criteria must be checked at screening and baseline; patients eligible for inclusion in this study must fulfill all the following criteria at both visits: The key eligibility criteria include (all the following must be met at the screening and randomization visits):
Male or female subjects≥18 years old or another legal maturity age according to the country of residence
Confirmed diagnosis of cancer (by histology or adequate imaging modality), other than basal-cell or squamous-cell carcinoma of the skin alone with one of the following:
   Active cancer, defined as either locally active, regionally invasive, or metastatic cancer at the time of randomization, and/or
   Currently receiving or having received anticancer therapy (radiotherapy, chemotherapy, hormonal therapy, any kind of targeted therapy or any other anticancer therapy) in the last 6 months.
Confirmed symptomatic or incidental proximal lower limb acute DVT (i.e., popliteal, femoral, iliac, and/or inferior vena cava vein thrombosis) and/or a confirmed symptomatic PE, or an incidental PE in a segmental, or larger pulmonary artery. Patients are eligible within 72 hours from diagnosis of the qualifying VTE.
Anticoagulation therapy with a therapeutic dose of DOAC for at least 6 months is indicated.
Able to provide written informed consent.

### Exclusion Criteria

Patients fulfilling any of the following criteria are not eligible for inclusion in this study:
Thrombectomy, insertion of a caval filter or use of a fibrinolytic agent to treat the current (index) occurrence of DVT and/or PE
More than 72 hours of pre-treatment with therapeutic doses of UFH, LMWH, fondaparinux, DOAC, or other anticoagulants
An indication to continue treatment with therapeutic doses of an anticoagulant other than that used for VTE treatment prior to randomization (e.g., atrial fibrillation, mechanical heart valve, prior VTE)
Platelet count<50,000/mm³
PE leading to hemodynamic instability (systolic blood pressure [BP]<90 mmHg or shock)
Acute ischemic or hemorrhagic stroke or intracranial hemorrhage within 4 weeks preceding screening
Brain trauma, or a cerebral or a spinal cord surgery in the 4 weeks preceding screening
Need for aspirin in a dosage of more than 100 mg/per day or any other antiplatelet agent alone or in combination with aspirin
Primary brain cancer or untreated intracranial metastases
Acute myeloid or lymphoid leukemia
Bleeding requiring medical attention at the time of randomization or in the preceding 4 weeks
Planned major surgery at baseline
Eastern Cooperative Oncology Group (ECOG) performance status of 3 or 4 at screening
Life expectancy<3 months at randomization
Calculated creatinine clearance (CrCl)<30 mL/min
Hemoglobin less than 8 g/dL
Acute hepatitis, chronic active hepatitis, liver cirrhosis; or an alanine aminotransferase level 3 times or more and/or bilirubin level 2 times or more higher the upper limit of the normal range in absence of clinical explanation
Uncontrolled hypertension (systolic BP>180 mm Hg or diastolic BP>100 mm Hg despite antihypertensive treatment)
Women of child-bearing potential (WOCBP) who are unwilling or unable to use highly effective contraceptive measures during the study from screening up to 3 days after last treatment of apixaban or 100 days after administration of Antibody 1
Sexually active males with sexual partners of childbearing potential must agree to use a condom or other reliable contraceptive measure up to 3 days after last treatment of apixaban or 100 days after administration of Antibody 1
Pregnant or breast-feeding women
Patients known to be receiving strong dual inducers or inhibitors of both CYP3A4 and P-gp
History of hypersensitivity to any of the study drugs (including apixaban) or its excipients, to drugs of similar chemical classes, or any contraindication listed in the label for apixaban
Subjects with any condition that as judged by the Investigator would place the subject at increased risk of harm if he/she participated in the study
Use of other investigational (not-registered) drugs within 5 half-lives prior to enrollment or until the expected pharmacodynamic effect has returned to baseline, whichever is longer. Participation in academic non-interventional or interventional studies, comprising testing different strategies or different combinations of registered drugs is permitted.

### Dosage and Administration

Patients will be randomized on Day 1 to one of the following treatment arms in a 1:1 ratio:
Antibody 1 150 mg iv followed by monthly 150 mg sc injection for 5 months (total duration of treatment is 6 months)
Apixaban 10 mg po bid for 7 days followed by 5 mg po bid for 5.75 months (total duration of treatment is 6 months)

Randomization into treatment groups will be stratified by region, cancer location (GI/GU vs other locations), and presentation of VTE (symptomatic vs incidental).

Once a subject is stratified and randomized, the subject will stay in that initial stratum, and randomized treatment group for analysis purposes even if their treatment or administered dose is subsequently adjusted.

### Dispensing the Study Medication

### Antibody 1

Each study site will be supplied with Antibody 1 as single-use vials. A unique kit number is printed on the vial label that corresponds to the Antibody 1 treatment arm. Investigator staff will identify the study drug vial to be used for the patient by contacting the IxRS and obtaining the kit number containing that vial.

At the Day 1 visit, Antibody 1 will be administered to the patient by qualified medical personnel at the study center via iv infusion over 60 minutes. For this first dose, patients must remain at the study center for at least 1 hour after administration of study drug to monitor for any infusion reactions, hypersensitivity reactions, or other AEs. Starting with the second dose (first sc dose), patients will return to the study center every month to receive sc administration of study drug and to complete visit procedures.

### Apixaban

Each study site will be supplied with cartons of apixaban 5 mg tablets. On Day 1, patients assigned to apixaban will be given a supply of apixaban and will be instructed to take two 10 mg doses that day approximately 12 hours apart. Patients will take 10 mg bid for 7 days followed by 5 mg bid for the remainder of the treatment period. If a patient was taking apixaban during the screening period, the total number of days taking 10 mg bid should not exceed 7 days. Throughout the study, apixaban will be dispensed at appropriate intervals to ensure patients have adequate quantities of study drug between study visits. Treatment compliance will be monitored during the study.

### Safety

### Physical Examination

A physical examination will include general appearance, skin, neck (including thyroid), eyes, ears, nose, throat, lungs, heart, abdomen, back, lymph nodes, extremities, musculoskeletal, vascular, and neurological assessments.

### Vital Signs

Vital signs will include the collection of oral body temperature (in ° C.), BP, and pulse.

### Laboratory Evaluations

Hemoglobin, hematocrit, red blood cell (RBC) count, mean corpuscular volume (MCV), mean corpuscular hemoglobin (MCH), mean corpuscular hemoglobin concentration (MCHC), red cell distribution width (RDW), white blood cell (WBC) count with differentials, and platelet count will be measured.

Sodium, potassium, creatinine, BUN/urea, uric acid, chloride, albumin, calcium, alkaline phosphatase, total bilirubin, bicarbonate/HCO3, AST, ALT, glucose, total cholesterol, and triglycerides will be measured. If total bilirubin is >2×ULN, direct and indirect reacting bilirubin should be differentiated.

Samples for standard aPTT as well as prothrombin time/international normalized ratio (PT/INR) will be collected at the visits as noted in the Assessment Schedule, as shown in Table 2.

Urine dipstick measurements for specific gravity, protein, glucose, and blood will be performed. Microscopy, WBC, RBC, and sediment will also be assessed in case of an abnormal dipstick test.

The primary safety variables are related to bleeding, are defined as secondary endpoints in the trial, and will be adjudicated by the CEC. Bleeding events will be classified as major bleeding, CRNM bleeding, and other bleeding according to the ISTH guidelines. These adjudicated bleeding events will be summarized as part of the evaluation of the secondary objective.

These bleeding events, regardless of adjudication, will also be included in the listings and analysis of all adverse effects. All information obtained on adverse events (AEs) will be displayed by treatment group and patient. AEs may be further displayed for the On-Treatment and Post-Treatment periods. AEs will be presented as a rate per 100 patient-years.

### Electrocardiogram (ECG)

ECGs will be performed at screening, baseline, EoT, and EoS visits. ECGs must be collected, interpreted locally by a qualified physician, appropriately signed, and archived at the study site.

### Clinical Events

### Efficacy

The independent blinded CEC will adjudicate and classify the following events:
All-cause death
All episodes of suspected DVT and PE recurrence
Other suspected arterial thromboembolic events (e.g., ischemic stroke, TIA, arterial embolic events, myocardial infarction [MI]), and other VTE such as leg distal DVT, upper extremity DVT, thoracic veins, cerebral intracranial or extracranial veins, splanchnic DVT and central venous line associated thrombosis

The primary outcome of the study (VTE recurrence) consists of the composite of confirmed (CEC adjudicated) VTE:
Confirmed, new symptomatic or incidental proximal DVT of the legs, iliac veins and/or IVC
Confirmed new symptomatic PE
Confirmed new incidental PE located in segmental or more proximal pulmonary arteries
Fatal PE (including unexplained death for which PE cannot be ruled out)

Confirmed arterial thromboembolic events (e.g., stroke, TIA, acute MI, and arterial embolic events) and confirmed other VTE (e.g., new leg distal DVT, upper extremity DVT, thoracic veins, cerebral intracranial or extracranial veins, splanchnic DVT, and central venous line associated thrombosis) are analyzed as study exploratory endpoints.

Incidental DVT or PE are thrombi that are detected during imaging testing performed for other reasons (e.g., CT for cancer staging) and not for suspicion of DVT or PE. A new thrombus incidentally detected in the IVC or iliac veins on an abdominal or pelvic CT is considered adequate to establish diagnosis of DVT recurrence. However, a new thrombus incidentally detected in the common femoral vein or more distal veins can only qualify for DVT recurrence if confirmed by CUS or venography.

### Bleeding

All suspected bleeding events either reported by the subject or observed by the Investigator should be recorded.

The details of all reported bleeding events will be submitted to the CEC as described in the endpoint reporting guidelines. These details may include, but are not limited to:
Location of the bleeding
Duration of the bleeding
Treatment of the bleeding event including notes or summaries of recommendations from a healthcare professional from whom medical treatment was obtained such as otolaryngology consults for ear, nose, or throat bleeds; urology consults for hematuria or urogenital tract bleeds; surgical consults for skin, soft tissue, or internal bleeds; gynecology consults for uterine or vaginal bleeds; neurology or neurosurgical consults for intracranial bleeds; or ophthalmology consults for ocular bleeds
Number of blood product transfusions
Magnitude of the bleeding (e.g., size if skin or subcutaneous hematoma)
Hemoglobin levels at the time of the bleeding event, lowest value, pre- and post-transfusion values, and after resolution of the bleeding event
Any diagnostic tests done to evaluate the bleeding such as endoscopy for GI bleeds
Any diagnostic imaging, e.g., x-ray, CT, MRI, or ultrasound, performed to evaluate the bleeding

### Major Bleeding Event

A major bleeding event will be confirmed when it meets at least 1 of the following:
a) Fatal bleeding
b) Symptomatic bleeding in a critical area or organ such as:
   Intracranial (epidural, subdural, subarachnoid, intra-cerebral, undetermined).
   Intraocular
   Intraspinal
   Intra-articular
   Intramuscular with compartment syndrome
   Pericardial
   Retroperitoneal
c) A clinically overt bleeding event
   associated with a fall in hemoglobin of 2.0 g/dL (>1.24 mMol/L) or more, or
   leading to a transfusion of ≥2 units of packed RBCs or whole blood

### Clinically Relevant Non-Major Bleeding Event

A bleeding event will be classified as a CRNM bleeding event if there is any sign or symptom of hemorrhage (e.g., more bleeding than would be expected for a clinical circumstance, including bleeding found by imaging alone) that does not fit the criteria for the definition of major bleeding but does meet at least one of the following criteria:
requiring medical intervention by a healthcare professional
leading to hospitalization or increased level of care
prompting a face-to-face (i.e., not just a telephone or electronic communication) evaluation.

### Minor (not Clinically Relevant) Bleeding Events

Other overt bleeding events that do not fulfill the criteria of a major bleeding event or a CRNM bleeding event will be classified as a minor bleeding event.

### No Bleeding

All other suspected bleeding events (e.g., decline in hemoglobin with no overt bleeding or incidental finding on imaging without further action as mentioned above will be classified as ‘no bleeding’.

### Other Assessments

### Pharmacokinetics

Blood samples for PK will be collected from a subset of patients assigned to Antibody 1 before administration of study drug and during some study visits as defined in the Assessment Schedule. Additional PK samples may be taken during unscheduled visits or during hospital admission for outcome events (VTE recurrence and bleeding events). Plasma concentrations of total Antibody 1 (i.e., bound or unbound to FXI) will be determined by a validated (LCMS/MS) method.

### Pharmacodynamic Assessments

Blood samples for PD assessments will be collected in a subset of patients at the timepoints defined in the Assessment Schedule. Additional PD samples may be taken during unscheduled visits or during hospital admission for outcome events (VTE recurrence and bleeding events).

PD biomarkers including but not limited to the following will be studied:
Free FXI—FXI that is not bound to Antibody 1 will be measured in plasma
Total FXI—FXI that is either bound to Antibody 1 or free will be measured in plasma
aPTT

Additionally, FXI:C may be measured in a subset of patients at selected sites, based on the site's access to appropriate −70° C./−80° C. freezers. FXI:C will be measured in plasma.

### Immunogenicity

An immunoassay-based method will be used to detect Antibody 1 ADA. Samples for ADA assessment will be collected before administration of study drug and on study visits as defined in the Assessment Schedule.

TABLE 2
Assessment Schedule

| | Screening | | | Treatment | | | | | | | Follow-up | |
|---|---|---|---|---|---|---|---|---|---|---|---|---|
| | Visit | | | | | | | | | | | |
| | | | | | Day | | | | | | Day 211 ¹³ | |
| | | | Day 8 | | 38 | | | | | | (EoT for | |
| | | Day | (phone | Day | (phone | Day | Day | Day | Day | Day 181 | Antibody 1) | Day 250 |
| | −3 to −1 | 1 | call) | 31 | call) | 61 | 91 | 121 | 151 | (EoT) ¹² | (phone call) | (EoS) |
| | Visit Window | | | | | | | | | | | |
| | | | ±3 | ±5 | ±5 | ±5 | ±5 | ±5 | ±5 | ±5 | ±10 | ±10 |
| Informed Consent | X | | | | | | | | X | | | |
| Informed consent for | | X | | | | | | | | | | |
| DNA sample collection | | | | | | | | | | | | |
| Demographic data | X | | | | | | | | | | | |
| Cancer status and | X | | | | | | | | | | | |
| location ¹ | | | | | | | | | | | | |
| Cancer treatment at | X | | | | | | | | | | | |
| presentation | | | | | | | | | | | | |
| Medical History | X | | | | | | | | | | | |
| Complete physical | | X | | | | | | | | | | |
| examination | | | | | | | | | | | | |
| Vital Signs | X | X | | X | | X | X | X | X | X | | X |
| Objectively confirmed | X | | | | | | | | | | | |
| VTE using imaging | | | | | | | | | | | | |
| modalities ² | | | | | | | | | | | | |
| ECG | X | | | | | | | | | X | | X |
| ECOG | X | | | | | | X | | | X | | X |
| Local aPTT, PT, INR | X | | | | | | | | | | | |
| Treatment with SoC | X | | | | | | | | | | | |
| Local safety lab | X | | | | | | | | | | | |
| assessments ³ | | | | | | | | | | | | |
| FSH to confirm post- | | X | | | | | | | | | | |
| menopausal status or | | | | | | | | | | | | |
| oophorectomy alone ³ | | | | | | | | | | | | |
| Central lab safety | | X | | X | | | X | | | X | | X |
| assessments ⁴ | | | | | | | | | | | | |
| Local lab urinary | | X | | | | | X | | | X | | X |
| dipstick (microscopy & | | | | | | | | | | | | |
| sediment if abnormal | | | | | | | | | | | | |
| dipstick test) | | | | | | | | | | | | |
| Pharmacogenomic | | X | | | | | | | | | | |
| Serum pregnancy test | | X ⁵ | | | | | | | | X ⁵ | | |
| (central lab) | | | | | | | | | | | | |
| Local lab serum or | X | | | X ⁵ | | | X ⁵ | | | | | X ⁵ |
| highly sensitive urine | | | | | | | | | | | | |
| pregnancy test ⁵ | | | | | | | | | | | | |
| Checking | X | X | | | | | | | | | | |
| Inclusion/exclusion | | | | | | | | | | | | |
| criteria | | | | | | | | | | | | |
| Randomization | | X | | | | | | | | | | |
| Antibody 1 | | X | | X | | X | X | X | X | | | |
| administration | | (iv) | | (sc) | | (sc) | (sc) | (sc) | (sc) | | | |
| Comparator | | X | | | | | | | | | | |
| administration | | | | | | | | | | | | |
| PK ^{6, 7} | | X ⁸ | | x | | | x | | | x | | x |
| Free XI, total FXI, | | X | | x | | | x | | | x | | x |
| FXI:C, aPTT ^{6, 7} | | | | | | | | | | | | |
| Additional BM of | | x | | x | | | x | | | | | |
| hypercoagulable state | | | | | | | | | | | | |
| (e.g., D-dimer, F1.2, | | | | | | | | | | | | |
| TAT, etc.) ⁶ | | | | | | | | | | | | |
| ADA ⁸ | | X | | | | | x | | | x | | x |
| HRQoL (EORTC QLQ | | X | | | | | x | | | x | | |
| C30, TSQM II) | | | | | | | | | | | | |
| HRQoL (EQ-5D-5L) ⁹ | | X | X ⁹ | X ⁹ | X ⁹ | X ⁹ | X | X ⁹ | X ⁹ | X | X ⁹ | X ⁹ |
| Health economic | | X | | X | | X | X | X | X | X | X | |
| outcome variables (VTE | | | | | | | | | | | | |
| and bleeding related | | | | | | | | | | | | |
| resource utilization) ¹⁰ | | | | | | | | | | | | |
| AE/SAE | X ¹¹ | X | | | | | | | | | | |
| Recording of | X | X | | X | | X | X | X | X | X | | X |
| concomitant therapy | | | | | | | | | | | | |
| (prescription drugs, | | | | | | | | | | | | |
| OTC, herbal, and non- | | | | | | | | | | | | |
| drug therapy) | | | | | | | | | | | | |
| VTE recurrence | | X | | X | | X | X | X | X | X | | X |
| Bleeding events | | X | | X | | X | X | X | X | X | | X |
| Transfusion assessment | | X | | X | | X | X | X | X | X | | X |

¹ to determine if patients are eligible for study participation;
² adjudication of index VTE is not required for randomization;
³ Local lab assessments include chemistry, hematology, LFT, creatinine clearance (Cockcroft-Gault equation), urinalysis, FSH to confirm post-menopausal status and, if WOCBP serum or highly sensitive urinary pregnancy test to determine eligibility;
⁴ Central lab safety assessments include a full panel of hematology, biochemistry, and urinalysis that will performed at baseline and selected subsequent visits to assess baseline and changes from baseline in laboratory parameters (not required to be eligible for study
participation);
⁵ to be performed in women of child bearing potential;
⁶ to be performed in a subset of patients randomized to Antibody 1; also, additional samples may be collected at unscheduled visits;
⁷ In patients receiving Antibody 1 only
⁸ Day 1 may include assessments before and 1 hour after Antibody 1 administration;
⁹ EQ-5D-5L will be recorded, whenever possible, at the subsequent visits following suspected VTE recurrence and suspected bleeding events;
¹⁰ this includes hospital stays, office visits and procedures and treatments to diagnose or to manage bleeding (e.g. endoscopy, arterial embolization) or VTE recurrence (e.g. intracaval filter)
¹¹ only SAEs will be reported, AEs are considered medical history,
¹² EoT visit procedures are also performed in case of PSDD;
¹³ The EoT visit for Antibody 1 may be conducted as actual visit in case of any suspicion of outcome events.
AE = adverse event,
aPTT = activated partial thromboplastin time;
F1.2 = prothrombin fragment 1.2;
ECG = electrocardiogram;
ECOG = Eastern Cooperative Oncology Group;
EORTC QLQ C30 = European Organization for the Research and Treatment of Cancer core Quality of Life Questionnaire;
EoS = end of study;
EoT = end of treatment;
FSH = follicle stimulating hormone;
FXI = factor XI;
GI = gastrointestinal;
FXI:C = FXI coagulation activity,
INR = international normalized ratio;
PD = pharmacodynamic;
PK = pharmacokinetic;
PSDD = permanent study drug discontinuation;
PT = prothrombin time;
SAE = serious adverse event;
TAT = thrombin-antithrombin;
TSQM II = Treatment Satisfaction Questionnaire for Medication

### Additional Biomarkers (Blood)

Additional blood samples will be collected at timepoints defined in the Assessment Schedule and stored for potential future exploratory analysis.

Additional biomarkers may include, but are not necessarily limited to:
D-dimer, F1.2, TAT

The list may be changed or expanded further, as it is recognized that more relevant or novel biomarkers may be discovered during the conduct of the study. This may be conducted in a subset of patients.

### Health-Related Quality of Life (HRQOL)

Three patient-reported outcomes (PRO) instruments will be used to measure HRQOL in countries and sites that are able to participate:
The EQ-5D-5L questionnaire will be used to collect information on the impact of the disease and its treatment on the patient's physical, emotional, and social well-being. The EQ-5D-5L is a self-administered, validated instrument that measures generic HRQOL across 5 dimensions: mobility, self-care, usual activities, pain/discomfort and anxiety/depression. For each dimension, there are 5 levels of response. In addition, the measure contains a visual analogue scale (VAS) scale that measures the respondent's overall health on a 0-100-point scale. This instrument has been widely used in clinical trials across a range of clinical conditions and among the general population (Berg et al. 2010).
The EORTC QLQ-C30 is a questionnaire developed to assess the quality of life of cancer patients. It is a 30-item questionnaire with a 4-point Likert scale for 28 items and a 7-point Likert scale for 2 items. It is a validated instrument that measures HRQOL. This instrument is available in over 100 languages (Farge et al. 2018).
The TSQM II measures patients' satisfaction with a provided treatment. It consists of 11 items, each on a 5-point Likert scale, measuring across the dimensions of treatment effectiveness, treatment-related side effects, treatment convenience, and satisfaction with treatment. This instrument is available in over 100 languages (Atkinson et al. 2004).

All PRO instruments will be administered electronically at the timepoints noted in the Assessment Schedule. PROs will be conducted in a subset of patients. Further, whenever possible, EQ-D-5L will be recorded at the subsequent visits following suspected VTE recurrence and suspected bleeding events.

### Exploratory DNA (Genetic) Samples

For patients who provided informed consent for this optional research, a blood sample for exploratory DNA will be taken on Day 1 or any time after informed consent is obtained. The collection of genetic samples is only applicable for those countries where the health authorities have approved of this testing.

Potential Risk Associated with SARS-COV2 (COVID-19)

The study treatments are not expected to alter the natural defense mechanisms or developing immunity to COVID-19. Anticoagulation therapy may have beneficial effects in preventing thromboembolic complications of COVID-19. Patients included in the study must apply social distancing and all other protective measures during the study; if COVID-19 is contracted, the patients should be managed according to usual medical practice. COVID-19 should be recorded in the AE eCRF.

If the COVID-19 pandemic prevents the patient from coming to planned or unscheduled visits, the patient should contact the Investigator. Every effort should be made to prevent treatment interruption and reporting of the study outcomes. Virtual and home visits may be considered as an option if necessary.

Vaccination for COVID-19 is permitted during the study.

### Study Completion and Discontinuation

Guidelines recommend anticoagulation therapy for 6 months in CAT (Streiff et al. 2018, Key et al. 2019, Khorana et al. 2018, Carrier et al 0.2018, NCCN Guidelines 2020). Intended treatment duration is 6 months in this study. Investigators should perform the EoT visit assessment at 6 months after randomization or earlier in case of PSDD. All patients will transition to a follow-up period of approximately 70 days after the EoT visit, where outcome events, other AEs and SAEs will continue to be reported and blood samples to assess ADA and PD tests will continue to be collected.

Some patients may require pursuing or resuming anticoagulation therapy in the follow-up period, Table 3 provides guidance for the initiation of anticoagulants other than the study treatments in the follow-up period.

TABLE 3
End of study transition guidelines

| Study | |
|---|---|
| treatment | Instructions |
| Apixaban | Patients could initiate DOACs, LMWH or fondaparinux 12 |
| | hours after the last dose of apixaban |
| Antibody 1 | Patients could initiate DOACs, LMWH treatment after |
| | 2 half-lives (~40 days) after the last dose of Antibody 1 |

The EoS visit will take place approximately 70 days after the EoT visit and will include collection of blood samples. This visit will assess patients for a general health and safety check prior to concluding their participation in the study.

### Discontinuation of Study Treatment

Discontinuation of study treatment for a patient occurs when study drug is permanently stopped earlier than the protocol planned duration. Study drug discontinuation can be initiated by either the patient or the Investigator.

Study treatment should be discontinued for any of the following:
Patient request
The Investigator judges that continued study drug administration is not in the best interest of the patient
Pregnancy
Received and/or having indication to continue prohibited medication

If a patient has what is expected to be PSDD, the patient should complete the EoT Visit procedures.

After study treatment discontinuation, at a minimum, in abbreviated visits, the following data should be collected at clinic visits or via telephone/video-call visits:
signs and symptoms suggestive of DVT, PE, bleeding, other thromboembolic events
new/concomitant treatments
AEs/SAEs

### Example 2—Treatment of Patients with Gastrointestinal or Genitourinary Cancer-Associated Thromboembolism with Antibody 1 Compared to Dalteparin

### Purpose and Rationale

The purpose of this study is to assess whether monthly treatment with Antibody 1 is non-inferior to daily administration of dalteparin in preventing VTE recurrence but is superior in the rate of bleeding in patients with gastrointestinal (GI)/genitourinary (GU) cancer and recently diagnosed VTE. This study will support worldwide registration of Antibody 1 for the treatment of CA VTE.

CAT occurs in an estimated 20% of cancer patients and is the second leading cause of death in patients with malignancies. Current treatments inhibit one or more factors in the coagulation cascade and while they effectively prevent or treat thrombosis, they also interfere with hemostasis resulting in a high risk of bleeding. The fear of bleeding and the lack of tolerability of available therapies leads to significant undertreatment and poor outcomes.

These issues are particularly challenging in patients with GI and GU cancers who are at an increased risk of bleeding. In fact, current guidelines recommend caution in using DOACs in patients with GI cancers because risk of bleeding is greater with DOACs compared to LMWH. While LMWH is the standard of care for treating CAT in patients with GI and GU tumors, this type of treatment requires daily injections for 6 months and patients treated with LMWH are less likely to continue therapy.

The goal of developing a new treatment for CAT, and especially patients with CAT who are at high risk for bleeding, is to maintain the same level of efficacy as current agents but with less bleeding and greater tolerability.

### Objectives and Endpoints

The primary objective of this study is to assess whether Antibody 1 is non-inferior to dalteparin for preventing VTE recurrence through 6 months post randomization in patients with GI or GU cancer and recently diagnosed VTE, with the endpoint being time to first event of centrally adjudicated VTE recurrence through 6 months.

The secondary objectives of this study are:
To assess whether Antibody 1 is superior to dalteparin for preventing the composite of major or CRNM bleeding through 6 months post randomization, with the endpoint being time to first event of centrally adjudicated VTE recurrence through 6 months
To assess whether Antibody 1 is superior to dalteparin on net clinical benefit defined as survival without VTE recurrence, or major or CRNM bleeding events through 6 months post randomization, with the endpoint being time to first event of VTE recurrence, ISTH-adjudicated major or ISTH-adjudicated CRNM bleeding events through 6 months
To assess whether Antibody 1 is superior to dalteparin for preventing VTE recurrence through 6 months post randomization, with the endpoint being time to first event of centrally adjudicated VTE recurrence through 6 months
To assess whether Antibody 1 is superior to dalteparin on the rate of permanent treatment discontinuation not due to death through 6 months post-randomization, with the endpoint being to assess whether Antibody 1 is superior to dalteparin on the rate of permanent treatment discontinuation not due to death through 6 months post-randomization
To assess whether Antibody 1 is superior to dalteparin for preventing occurrence of CRNM bleeding events through 6 months post randomization, with the endpoint being time to first event of ISTH-adjudicated CRNM bleeding events through 6 months
To assess whether Antibody 1 is superior to dalteparin for preventing occurrence of major bleeding events through 6 months post randomization, with the endpoint being Time to first event of ISTH-adjudicated major bleeding events through 6 months
To assess whether Antibody 1 is superior to dalteparin for preventing occurrence of the composite of GI major and GI CRNM bleeding through 6 months post randomization, with the endpoint being time to first event of GI ISTH-adjudicated major and GI CRNM bleeding events through 6 months
To evaluate safety and tolerability of Antibody 1 relative to dalteparin through 6 months post randomization and to assess incidence rate of injection site reactions, hypersensitivity reactions, and immunogenicity in patients treated with Antibody 1, with the endpoints being all-cause death, vascular death, serious adverse events, adverse events leading to drug discontinuation, other adverse events, abnormal lab tests, etc. presented as rate per 100 patient-years; and, for patients treated with Antibody 1:
   Percentage of patients with injection site reactions
   Percentage of patients with injection site reactions by severity status
   Percentage of patients with hypersensitivity reactions
   Percentage of patients with hypersensitivity reactions by severity status
   Percentage of patients with ADA formation
   Percentage of patients with persistent ADA formation
   Percentage of patients with neutralizing antibody (NAb) formation.

The exploratory objectives of this study are:
To evaluate the effect of Antibody 1 relative to dalteparin on the composite of VTE recurrence, major bleeding events or all-cause death, with the endpoint being time to first event of the composite endpoint of VTE recurrence, major bleeding, or all-cause death
To assess the effect of Antibody 1 relative to dalteparin on arterial thromboembolic events (e.g., stroke, myocardial infarction, arterial embolic events), with the endpoint being time to first event of the composite endpoint of ischemic stroke, or myocardial infarction or arterial embolic events
To assess the effect of Antibody 1 relative to dalteparin on venous thromboembolic events other than events qualifying for VTE recurrence (e.g., leg distal DVT, upper extremity DVT, thoracic veins, intracranial or extracranial cerebral veins, splanchnic DVT and central venous line associated thrombosis), with the endpoints being time to first event of the composite endpoint of DVT recurrence or other venous thromboembolic events and time to the first event of the following venous thromboembolic events: leg distal DVT, upper extremity DVT, thoracic veins, intracranial or extracranial cerebral veins, splanchnic DVT or central venous line associated thrombosis
To assess the effect of Antibody 1 relative to dalteparin on the number and duration of temporary treatment interruptions which are not due to procedures or bleeding events, with the endpoints being number of temporary treatment interruptions which are not due to procedures or bleeding and total duration of temporary treatment interruptions which are not due to procedures or bleeding
To assess efficacy and safety of Antibody 1 relative to dalteparin in predefined subgroups of patients (e.g., gender, age, BMI, ethnicity, cancer location, incidental vs. symptomatic VTE, presence or absence of PE, days on SoC prior to randomization, ECOG performance status), with the endpoints being time to first event of centrally adjudicated VTE recurrence through 6 months in predefined subgroups of patients and time to first event of ISTH-defined major and CRNM bleeding events through 6 months in the predefined subgroups of patients
To assess the effect of Antibody 1 relative to dalteparin on HRQOL at 3 and 6 months and to assess changes over time in EQ-5D-5L in patients who present suspected VTE recurrence and suspected bleeding events, with the endpoints being change from baseline in the overall score and score by domain of EQ-5D-5L questionnaire, EORTC QLQ C30 questionnaire, and TSQM II questionnaire; and serial changes over time in EQ-5D-5L questionnaire following suspected VTE recurrence and bleeding events
To assess the PK of Antibody 1 in a subset of patients with the endpoint being random, peak and trough Antibody 1 plasma concentrations
To assess the pharmacodynamics (free and total FXI, FXI:C aPTT) of Antibody 1 in a subset of patients, with the endpoint being free and total FXI, FXI:C and aPTT at indicated time points
To assess the effect on Antibody 1 relative to dalteparin on exploratory biomarkers of hypercoagulable state (potentially including but not limited to d-dimer, F1.2, TAT) in subset of patients, with the endpoint being changes from baselines in biomarkers of hypercoagulable state which may include d-dimer, F1.2 and TAT between treatment arms
VTE cost effectiveness analysis (e.g., hospital stay, transfusion, procedures to stop bleeding or to manage VTE, physicians' office visits), with the endpoint being healthcare resource utilization
DNA assessments to explore whether individual genetic variation in genes relating to the drug target pathway or other relevant genetic pathways confer differential responses to Antibody 1, with the endpoint being exploratory evaluation of the association between gene polymorphisms and safety, efficacy, PK, and PD response

### Study Design

This is a randomized, open-label, blinded endpoint evaluation, phase 3 study comparing the effect of Antibody 1 relative to dalteparin on VTE recurrence in patients with GI or GU cancer.

Randomization into treatment groups will be stratified by region, cancer location (GI or GU cancers), and symptomatic vs incidental thromboembolic events.

The study is comprised of 3 periods: 1) screening (up to 3 days [72 hours]), 2) study drug treatment period through the End of Treatment (EoT) visit, and 3) follow up period through the End of Study (EoS) visit.

### Population

Adult male or female subjects are eligible for study participation if they have confirmed (histology, adequate imaging modality) unresectable GI or GU cancer prior to randomization and presentation of acute VTE (within 72 hours of diagnosis of the qualifying VTE), for which long-term treatment with LMWH is indicated; approximately 1020 patients will be randomized in this study.

### Inclusion Criteria

Inclusion and exclusion criteria must be checked at screening and baseline; patients eligible for inclusion in this study must fulfill all the following criteria at both visits:
Male or female subjects≥18 years old or other legal maturity age according to the country of residence
Confirmed GI (colorectal, pancreatic, gastric, esophageal, gastro-esophageal junction or hepatobiliary) or confirmed GU (renal, ureteral, bladder, prostate, or urethra) cancer prior to randomization if:
   Unresectable, locally advanced, metastatic, or non-metastatic GI/GU cancer and
   No intended curative surgery during the study
Confirmed symptomatic or incidental proximal lower limb acute DVT (i.e., popliteal, femoral, iliac, and/or inferior vena cava [IVC] thrombosis) and/or a confirmed symptomatic or incidental PE of a segmental, or larger pulmonary artery, and/or a confirmed symptomatic PE, or an incidental PE in a segmental, or larger pulmonary artery. Patients are eligible within 72 hours from diagnosis of the qualifying VTE
Anticoagulation therapy with LMWH for at least 6 months is indicated
Able to provide written informed consent

### Exclusion Criteria

Patients fulfilling any of the following criteria are not eligible for inclusion in this study:
Thrombectomy, insertion of a caval filter or use of a fibrinolytic agent to treat the current (index) DVT and/or PE
More than 72 hours of pre-treatment with therapeutic doses of UFH, LMWH, or other anticoagulants
An indication to continue treatment with therapeutic doses of an anticoagulant other than that for VTE treatment prior to randomization (e.g., AF, mechanical heart valve, prior VTE)
PE leading to hemodynamic instability (blood pressure [BP]<90 mmHg or shock)
Acute ischemic or hemorrhagic stroke or intracranial hemorrhage within 4 weeks of screening
Brain trauma or a cerebral or spinal cord surgery within 4 weeks of screening
Need for aspirin in a dosage of >100 mg/day or any other antiplatelet agent alone or in combination with aspirin
Bleeding requiring medical attention at the time of randomization or within the preceding 4 weeks
Planned major surgery at baseline
History of heparin-induced thrombocytopenia
Primary brain cancer or untreated intracranial metastasis
Eastern Cooperative Oncology Group (ECOG) performance status of 3 or 4 at screening
Life expectancy<3 months at randomization
Calculated creatinine clearance (CrCl)<30 mL/min (Cockcroft-Gault equation)
Platelet count<50,000/mm³
Hemoglobin<8 g/dL
Acute hepatitis, chronic active hepatitis, liver cirrhosis; or an alanine aminotransferase (ALT)≥3× and/or bilirubin≥2× upper limit of normal (ULN) in absence of clinical explanation
Uncontrolled hypertension (systolic BP>180 mm Hg or diastolic BP>100 mm Hg) despite antihypertensive treatment
Women of child-bearing potential (WOCBP) who are unwilling or unable to use highly effective contraceptive measures during the study from screening up to 3 days after last treatment of dalteparin or 100 days after administration of Antibody 1
Sexually active males with sexual partners of childbearing potential must agree to use a condom or other reliable contraceptive measure up to 3 days after last treatment of dalteparin or 100 days after administration of Antibody 1.
Pregnant or breast-feeding women
History of hypersensitivity to any of the study drugs (including dalteparin) or its excipients, to drugs of similar chemical classes, or any contraindication listed in the label for dalteparin
Subjects with any condition that in the Investigator's judgement would place the subject at increased risk of harm if he/she participated in the study
Use of other investigational (not-registered) drugs within 5 half-lives prior to enrollment or until the expected PD effect has returned to baseline, whichever is longer. Participation in academic non-interventional studies or interventional studies testing different strategies or different combinations of registered drugs is permitted.

### Dosage and Administration

Patients will be randomized on Day 1 to one of the following treatment arms in a 1:1 ratio:
Antibody 1 150 mg iv followed by monthly 150 mg sc injection for 5 months (total duration of treatment is 6 months)
Dalteparin 200 IU/kg/day sc for 1 month followed by 150 IU/kg/day sc for 5 months. The maximum daily dose allowed for dalteparin is 18,000 IU (total duration of treatment is 6 months).

Randomization into treatment groups will be stratified by region, cancer location (GI vs GU) and presentation of VTE (symptomatic vs incidental).

Once a subject is stratified and randomized, the subject will stay in that initial stratum, and randomized treatment group for analysis purposes even if their treatment or administered dose is subsequently adjusted.

### Safety

### Physical Examination

A physical examination will include general appearance, skin, neck (including thyroid), eyes, ears, nose, throat, lungs, heart, abdomen, back, lymph nodes, extremities, musculoskeletal, vascular, and neurological assessments.

### Vital Signs

Vital signs will include the collection of oral body temperature (in ° C.), BP, and pulse.

### Laboratory Evaluations

Hemoglobin, hematocrit, red blood cell (RBC) count, mean corpuscular volume (MCV), mean corpuscular hemoglobin (MCH), mean corpuscular hemoglobin concentration (MCHC), red cell distribution width (RDW), white blood cell (WBC) count with differentials, and platelet count will be measured.

Sodium, potassium, creatinine, BUN/urea, uric acid, chloride, albumin, calcium, alkaline phosphatase, total bilirubin, bicarbonate/HCO3, AST, ALT, glucose, total cholesterol, and triglycerides will be measured. If total bilirubin is ≥2×ULN, direct and indirect reacting bilirubin should be differentiated.

Samples for standard aPTT as well as prothrombin time/international normalized ratio (PT/INR) will be collected at the visits noted in the Assessment Schedule.

Urine dipstick measurements for specific gravity, protein, glucose, and blood will be performed. Microscopy, WBC, RBC, and sediment will also be assessed in case of an abnormal dipstick test.

### Electrocardiogram (ECG)

ECGs will be performed at screening, baseline, EoT, and EoS visits. ECGs must be collected, interpreted locally by a qualified physician, appropriately signed, and archived at the study site.

### Clinical Endpoints

### Efficacy

The independent blinded CEC will adjudicate and classify the following events:
All-cause death
All episodes of suspected DVT and PE recurrence
Other suspected arterial thromboembolic events (e.g., ischemic stroke, TIA, arterial embolic events, myocardial infarction [MI]), and other VTEs such as leg distal DVT, upper extremity DVT, thoracic veins, cerebral intracranial or extracranial veins, splanchnic DVT, and central venous line associated thrombosis

The primary outcome of the study (VTE recurrence) consists of the composite of confirmed (CEC adjudicated) VTE events:
Confirmed new symptomatic or incidental proximal DVT of the legs, iliac veins and/or IVC
Confirmed new symptomatic PE
Confirmed new incidental PE located in segmental or more proximal pulmonary arteries
Fatal PE (including unexplained death for which PE cannot be ruled out)

Confirmed arterial thromboembolic events (e.g., stroke, TIA, acute MI, and arterial embolic events) and confirmed other VTE (e.g., new leg distal DVT, upper extremity DVT, thoracic veins, cerebral intracranial or extracranial veins, splanchnic DVT, and central venous line associated thrombosis) are analyzed as study exploratory endpoints.

Incidental DVT or PE are thrombi that are detected during imaging testing performed for other reasons (e.g., CT for cancer staging) and not for suspicion of DVT or PE. A new thrombus incidentally detected in the IVC or iliac veins on an abdominal or pelvic CT is considered adequate to establish diagnosis of DVT recurrence. However, a new thrombus incidentally detected in the common femoral vein or more distal veins can only qualify for DVT recurrence if confirmed by CUS or venography.

Index VTE and outcome events occurring up to the EoS visit will be adjudicated in all randomized patients.

### Bleeding

All suspected bleeding events either reported by the subject or observed by the Investigator should be recorded.

The details of all reported bleeding events will be submitted to the CEC as described in the endpoint reporting guidelines. These details may include, but are not limited to,
Location of the bleeding
Duration of the bleeding
Treatment of the bleeding event including notes or summaries of recommendations from a healthcare professional from whom medical treatment was obtained such as otolaryngology consults for ear, nose, or throat bleeds; urology consults for hematuria or urogenital tract bleeds; surgical consults for skin, soft tissue, or internal bleeds; gynecology consults for uterine or vaginal bleeds; neurology or neurosurgical consults for intracranial bleeds; or ophthalmology consults for ocular bleeds
Number of blood product transfusions
Magnitude of the bleeding (e.g., size if skin or subcutaneous hematoma)
Hemoglobin levels at the time of the bleeding event, lowest value, pre- and post-transfusion values, and after resolution of the bleeding event
Any diagnostic tests done to evaluate the bleeding such as endoscopy for GI bleeds
Any diagnostic imaging, e.g., x-ray, CT, MRI, or ultrasound, performed to evaluate the bleeding
Any other information that could be useful to the CEC in adjudicating the bleeding event

Overt bleeding events will be centrally adjudicated by the CEC. The CEC will classify bleeding events in accordance with the International Society on Thrombosis and Haemostasis (ISTH) definitions and guidance (Kaatz et al. 2015):

### Major Bleeding Event

A major bleeding event will be confirmed when it meets at least 1 of the following:
a) Fatal bleeding
b) Symptomatic bleeding in a critical area or organ such as:
   Intracranial (epidural, subdural, subarachnoid, intra-cerebral, undetermined)
   Intraocular
   Intraspinal
   Intra-articular
   Intramuscular with compartment syndrome
   Pericardial
   Retroperitoneal
c) A clinically overt bleeding event
   associated with a fall in hemoglobin of ≥2.0 g/dL (≥1.24 mMol/L), or
   leading to a transfusion of ≥2 units of packed RBCs or whole blood

### Clinically Relevant Non-Major Bleeding Event

A bleeding event will be classified as a CRNM bleeding event if there is any sign or symptom of hemorrhage (e.g., more bleeding than would be expected for a clinical circumstance, including bleeding found by imaging alone) that does not fit the criteria for the definition of major bleeding but does meet at least 1 of the following criteria:
requiring medical intervention by a healthcare professional
leading to hospitalization or increased level of care
prompting a face to face (i.e., not just a telephone or electronic communication) evaluation

### Minor (not Clinically Relevant) Bleeding Events

Other overt bleeding events that do not fulfill the criteria of a major bleeding event or a CRNM bleeding event will be classified as a minor bleeding event.

### No Bleeding

All other suspected bleeding events (e.g., decline in hemoglobin with no overt bleeding or incidental finding on imaging without further action as mentioned above) will be classified as ‘no bleeding’.

### Other Assessments

### Pharmacokinetics

Blood samples for PK will be collected from a subset of patients assigned to Antibody 1 before administration of study drug and during some study visits as defined in the Assessment Schedule. Additional PK samples may be taken during unscheduled visits or during hospital admission for outcome events (VTE recurrence and bleeding events). Plasma concentrations of total Antibody 1 (i.e., bound or unbound to FXI) will be determined by a validated LCMS/MS method.

### Pharmacodynamic Assessments

Blood samples for PD assessments will be collected in a subset of patients at the timepoints defined in the Assessment Schedule. Additional PD samples may be taken during unscheduled visits or during hospital admission for outcome events (VTE recurrence and bleeding events).

PD biomarkers including, but not limited to the following, will be studied:
Free FXI—FXI that is not bound to Antibody 1 will be measured in plasma
Total FXI—FXI that is either bound to Antibody 1 or free will be measured in plasma
aPTT

Additionally, FXI:C may be measured in a subset of patients at selected sites, based on the site's access to appropriate −70° C./−80° C. freezers. FXI:C will be measured in plasma.

### Immunogenicity

An immunoassay-based method will be used to detect Antibody 1 ADA. Blood samples for IG will be collected before administration of study drug and on study visits as defined in the Assessment Schedule shown in Table 4.

TABLE 4
Assessment Schedule

| | Screening | | | Treatment | | | | | | | Follow-up | |
|---|---|---|---|---|---|---|---|---|---|---|---|---|
| | Visit | | | | | | | | | | | |
| | | | | | Day | | | | | | Day 211 ¹³ | |
| | | | Day 8 | | 38 | | | | | | (EoT for | |
| | | Day | (phone | Day | (phone | Day | Day | Day | Day | Day 181 | Antibody 1) | Day 250 |
| | −3 to −1 | 1 | call) | 31 | call) | 61 | 91 | 121 | 151 | (EoT) ¹² | (phone call) | (EoS) |
| | Visit Window | | | | | | | | | | | |
| | | | ±3 | ±5 | ±5 | ±5 | ±5 | ±5 | ±5 | ±5 | ±10 | ±10 |
| Informed Consent | X | | | | | | | | | | | |
| Informed consent for | | X | | | | | | | | | | |
| DNA sample collection | | | | | | | | | | | | |
| Demographic data | X | | | | | | | | | | | |
| Cancer status and | X | | | | | | | | | | | |
| location ¹ | | | | | | | | | | | | |
| Cancer treatment at | X | | | | | | | | | | | |
| presentation | | | | | | | | | | | | |
| Medical History | X | | | | | | | | | | | |
| Complete physical | | X | | | | | | | | | | |
| examination | | | | | | | | | | | | |
| Vital Signs | X | X | | X | | X | X | X | X | X | | X |
| Objectively confirmed | X | | | | | | | | | | | |
| VTE using imaging | | | | | | | | | | | | |
| modalities ² | | | | | | | | | | | | |
| ECG | X | | | | | | | | | X | | X |
| ECOG | X | | | | | | X | | | X | | X |
| Local aPTT, PT, INR | X | | | | | | | | | | | |
| Treatment with SoC | X | | | | | | | | | | | |
| Local safety lab | X | | | | | | | | | | | |
| assessments ³ | | | | | | | | | | | | |
| FSH to confirm post- | | X | | | | | | | | | | |
| menopausal status or | | | | | | | | | | | | |
| oophorectomy alone ³ | | | | | | | | | | | | |
| Central lab safety | | X | | X | | | X | | | X | | X |
| assessments ⁴ | | | | | | | | | | | | |
| Local lab urinary | | X | | | | | X | | | X | | X |
| dipstick (microscopy & | | | | | | | | | | | | |
| sediment if abnormal | | | | | | | | | | | | |
| dipstick test) | | | | | | | | | | | | |
| Pharmacogenomic | | X | | | | | | | | | | |
| Serum pregnancy test | | X ⁵ | | | | | | | | X ⁵ | | |
| (central lab) | | | | | | | | | | | | |
| Local lab serum or | X | | | X ⁵ | | | X ⁵ | | | | | X ⁵ |
| highly sensitive urine | | | | | | | | | | | | |
| pregnancy test ⁵ | | | | | | | | | | | | |
| Checking | X | X | | | | | | | | | | |
| Inclusion/exclusion | | | | | | | | | | | | |
| criteria | | | | | | | | | | | | |
| Randomization | | X | | | | | | | | | | |
| Antibody 1 | | X | | X | | X | X | X | X | | | |
| administration | | (iv) | | (sc) | | (sc) | (sc) | (sc) | (sc) | | | |
| Comparator | | X | | | | | | | | | | |
| administration | | | | | | | | | | | | |
| PK ^{6, 7} | | X ⁸ | | X | | | X | | | X | | X |
| Free XI, total FXI, | | X | | X | | | X | | | X | | X |
| FXI:C, aPTT ^{6, 7} | | | | | | | | | | | | |
| Additional BM of | | X | | X | | | X | | | | | |
| hypercoagulable state | | | | | | | | | | | | |
| (e.g., D-dimer, F1.2, | | | | | | | | | | | | |
| TAT, etc.) ⁶ | | | | | | | | | | | | |
| ADA ⁸ | | X | | | | | X | | | X | | X |
| HRQoL (EORTC QLQ | | X | | | | | X | | | X | | |
| C30, TSQM II) | | | | | | | | | | | | |
| HRQoL (EQ-5D-5L) ⁹ | | X | X ⁹ | X ⁹ | X ⁹ | X ⁹ | X | X ⁹ | X ⁹ | X | X ⁹ | X ⁹ |
| Health economic | | X | | X | | X | X | X | X | X | X | |
| outcome variables (VTE | | | | | | | | | | | | |
| and bleeding related | | | | | | | | | | | | |
| resource utilization) ¹⁰ | | | | | | | | | | | | |
| AE/SAE | X ¹¹ | X | | | | | | | | | | |
| Recording of | X | X | | X | | X | X | X | X | X | | X |
| concomitant therapy | | | | | | | | | | | | |
| (prescription drugs, | | | | | | | | | | | | |
| OTC, herbal, and non- | | | | | | | | | | | | |
| drug therapy) | | | | | | | | | | | | |
| VTE recurrence | | X | | X | | X | X | X | X | X | | X |
| Bleeding events | | X | | X | | X | X | X | X | X | | X |
| Transfusion assessment | | X | | X | | X | X | X | X | X | | X |

¹ to determine if patients are eligible for study participation;
² adjudication of index VTE is not required for randomization;
³ Local lab assessments include chemistry, hematology, LFT, creatinine clearance (Cockcroft-Gault equation), urinalysis, FSH to confirm post-menopausal status and, if WOCBP serum or highly sensitive urinary pregnancy test to determine eligibility;
⁴ Central lab safety assessments include a full panel of hematology, biochemistry, and urinalysis that will performed at baseline and selected subsequent visits to assess baseline and changes from baseline in laboratory parameters (not required to be eligible for study participation);
⁵ to be performed in women of child bearing potential;
⁶ to be performed in a subset of patients randomized to Antibody 1; also, additional samples may be collected at unscheduled visits;
⁷ In patients receiving Antibody 1 only;
⁸ Day 1 may include assessments before and 1 hour after Antibody 1 administration;
⁹ EQ-5D-5L will be recorded, whenever possible, at the subsequent visits following suspected VTE recurrence and suspected bleeding events;
¹⁰ this includes hospital stays, office visits and procedures and treatments to diagnose or to manage bleeding (e.g. endoscopy, arterial embolization) or VTE recurrence (e.g. intracaval filter);
¹¹ only SAEs will be reported, AEs are considered medical history;
¹² EoT visit procedures are also performed in case of PSDD;
¹³ The EoT visit for Antibody 1 may be conducted as actual visit in case of any suspicion of outcome events.
AE = adverse evet,
aPTT = activated partial thromboplastin time;
F1.2 = prothrombin fragment 1.2;
ECG = electrocardiogram;
ECOG = Eastern Cooperative Oncology Group;
EORTC QLQ C30 = European Organization for the Research and Treatment of Cancer core Quality of Life Questionnaire;
EoS = end of study;
EoT = end of treatment;
FSH = follicle stimulating hormone;
FXI = factor XI;
GI = gastrointestinal;
FXI:C = FXI coagulation activity,
INR = international normalized ratio;
PD = pharmacodynamic;
PK = pharmacokinetic;
PSDD = permanent study drug discontinuation;
PT = prothrombin time;
SAE = serious adverse event;
TAT = thrombin-antithrombin;
TSQM II = Treatment Satisfaction Questionnaire for Medication

### Additional Biomarkers (Blood)

Additional blood samples will be collected at timepoints defined in the Assessment Schedule and stored for potential future exploratory analysis.

Additional biomarkers may include, but are not necessarily limited to:
D-dimer, F1.2, TAT

The list may be changed or expanded further, as it is recognized that more relevant or novel biomarkers may be discovered during the conduct of the study. This may be conducted in a subset of patients.

### Health-Related Quality of Life (HRQOL)

Three patient-reported outcomes (PRO) instruments will be used to measure HRQOL in countries and sites that are able to participate:
The EQ-5D-5L questionnaire will be used to collect information on the impact of the disease and its treatment on the patient's physical, emotional, and social well-being. The EQ-5D-5L is a self-administered, validated instrument that measures generic HRQOL across 5 dimensions: mobility, self-care, usual activities, pain/discomfort, and anxiety/depression. For each dimension, there are 5 levels of response. In addition, the measure contains a visual analogue scale (VAS) scale that measures the respondent's overall health on a 0-100-point scale. This instrument has been widely used in clinical trials across a range of clinical conditions and among the general population (Berg et al. 2010).
The EORTC QLQ-C30 is a questionnaire developed to assess the quality of life of cancer patients. It is a 30-item questionnaire with a 4-point Likert scale for 28 items and a 7-point Likert scale for 2 items. It is a validated instrument that measures HRQOL. This instrument is available in over 100 languages (Farge et al. 2018).
The TSQM II measures patients' satisfaction with a provided treatment. It consists of 11 items, with a 5-point Likert scale, measuring across the dimensions of treatment effectiveness, treatment-related side effects, treatment convenience, and satisfaction with treatment. This instrument is available in over 100 languages (Atkinson et al. 2004).

All PROs will be administered electronically at the timepoints noted in the Assessment Schedule. PROs will be conducted in a subset of patients. Further, whenever possible, EQ-D-5L will be recorded at the subsequent visits following suspected VTE recurrence and suspected bleeding events.

### Exploratory DNA (Genetic) Samples

For patients who provided consent for this optional research, a blood sample for exploratory DNA will be taken on Day 1 or any time after informed consent is obtained. The collection of genetic samples is only applicable in those countries where the health authorities have approved of this testing.

Potential Risk Associated with SARS-COV2 (COVID-19)

The study treatments are not expected to alter the natural defense mechanisms or developing immunity to COVID-19. Anticoagulation therapy may have beneficial effects in preventing thromboembolic complications of COVID-19. Patients included in the study must apply social distancing and all other protective measures during the study; if COVID-19 is contracted, the patient should be managed according to usual medical practice. COVID-19 should be recorded in the AE eCRF.

If the COVID-19 pandemic prevents the patient from coming to planned or unscheduled visits, the patient should contact the Investigator. Every effort should be made to prevent treatment interruption and ensure reporting of the study outcomes. Virtual and home visits may be considered an option if necessary.

Vaccination for COVID-19 is permitted during the study.

### Study Completion and Discontinuation

### Study Completion and Post-Study Treatment

Guidelines recommend anticoagulation therapy for 6 months in CAT (Streiff et al. 2018, Key et al. 2019, Khorana et al. 2018, Carrier et al. 2018, NCCN Guidelines 2020). Intended treatment duration is 6 months in this study. The Investigator should perform the EoT visit assessment at 6 months after randomization or earlier in case of PSDD. All patients will transition to a follow up period of approximately 70 days after the EoT visit, where outcome events, other AEs, and SAEs will continue to be reported and blood samples to assess ADA and PD tests will continue to be collected.

Some patients may require initiating or resuming anticoagulation therapy in the follow up period. Table 5 provides guidance for the initiation of anticoagulants other than the study treatments in the follow up period.

TABLE 5
End of study transition guidelines

| Study | |
|---|---|
| treatment | Instructions |
| Dalteparin | Patients could initiate LMWH, fondaparinux or other |
| | therapy 24 hours after the last dose of dalteparin |
| Antibody 1 | Patients could initiate DOACs, LMWH treatment after |
| | 2 half-lives (~40 days) after the last dose of Antibody 1 |

The EoS visit will take place approximately 70 days after the EoT visit and will include collection of blood samples. This visit will assess patients for a general health and safety check prior to concluding their participation in the study.

### Discontinuation of Study Treatment

Discontinuation of study treatment for a patient occurs when study drug is permanently stopped earlier than the protocol planned duration. Study drug discontinuation can be initiated by either the patient or the Investigator.

Study treatment should be discontinued for any of the following:
Patient request
The investigator judges that continued study drug administration is not in the best interest of the patient
Pregnancy
Received and/or having indication to continue forbidden therapy

If a patient has what is expected to be PSDD, the patient should complete the EoT.

After study treatment discontinuation, at a minimum, in abbreviated visits, the following data should be collected at clinic visits or via telephone/video-call visits:
signs and symptoms suggestive of DVT, PE, bleeding, other thromboembolic events
new/concomitant treatments
AEs/SAEs

If the patient cannot or is unwilling to attend any visit(s), the site staff should maintain regular telephone contact with the patient, or with a person pre-designated by the patient.

### Example 3—Antibody 1 in a Baboon Model of Vascular Graft Thrombosis

This Example aims to test whether Antibody 1 is effective in halting clot formation and downstream growth when administered before or during active clot formation in an established baboon femoral arterio-venous (AV) shunt model. The baboon model is described, for example, in Gruber et al. Blood, 1989 Feb. 15; 73(3):639-42 and Crosby et al. Arterioscler Thromb Vasc Biol, 2013 July; 33(7):1670-8.

Juvenile male baboons (8-10 kg in weight) had a chronic femoral AV shunt put in place. Three animals were tested for pharmacodynamics and in AV-shunt experiments; one animal was tested for pharmacodynamics only. Seven experiments: two untreated controls, one treatment of Antibody 1 at 1 mg/kg intravenously administered 30 minutes after clot initiation, one prevention 24 hours after Antibody 1, one prevention 48 hours after Antibody 1, and two prevention 144 to 216 hours after Antibody 1.

Platelet and fibrin deposition inside and distal to collagen- or collagen+tissue factor (TF)-coated vascular grafts were measured. ¹¹¹Indium-labeled platelets and ¹²⁵Iodine-labeled fibrinogen were administered to the baboons prior to experiments. The perfused collagen-coated graft produced a progressively propagating distal thrombus in the shunt (the tail segment) which is the region of interest for measuring Antibody 1 effect on thrombus growth The collagen+TF-coated graft is the region of interest for measuring the Antibody 1 effect on hemostasis. Additionally, real-time gamma camera acquisition of radioactivity within the thrombogenic device was done to determine the amount and the rate of platelets deposition. After 120 minutes perfusion, grafts were removed and stored for subsequent measurement of ¹²⁵iodine radioactivity after the decay of ¹¹¹indium to determine fibrin deposition.

Additionally, activated partial thromboplastin time (aPTT) and prothrombin time (PT) were measured prior to and during the thrombosis experiments and followed until return to baseline levels. Bleeding time (BT) and bleeding volume (BV) were assessed using FDA approved method for use in patients (Surgicutt® device, International Technidyne, Piscataway, NJ). Two BT/BV measurements were taken prior to and during each thrombosis experiment.

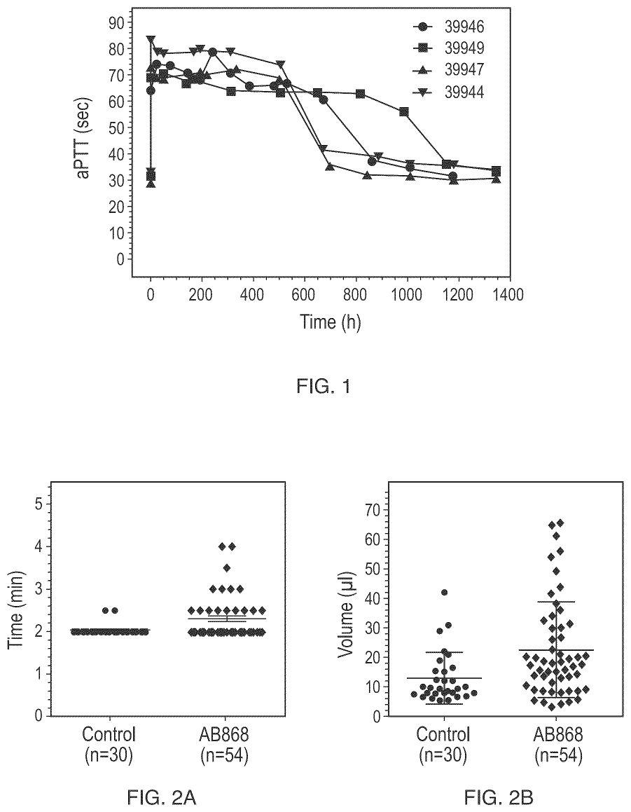

A 1 mg/kg iv dose of Antibody 1 resulted in immediate aPTT prolongation of approximately 2 to 2.5-fold from baseline levels. Prolongation of aPTT persisted up to 600 hours after drug administration, then slowly returned to baseline (FIG. 1). No change in PT was observed following Antibody 1 administration.

A small increase in mean bleeding time (FIG. 2A) and bleeding volume (FIG. 2B) of about 10% to 15% increase was observed after administration of Antibody 1. This increase is not believed to be clinically relevant due to existing clinical trial safety data for Antibody 1.

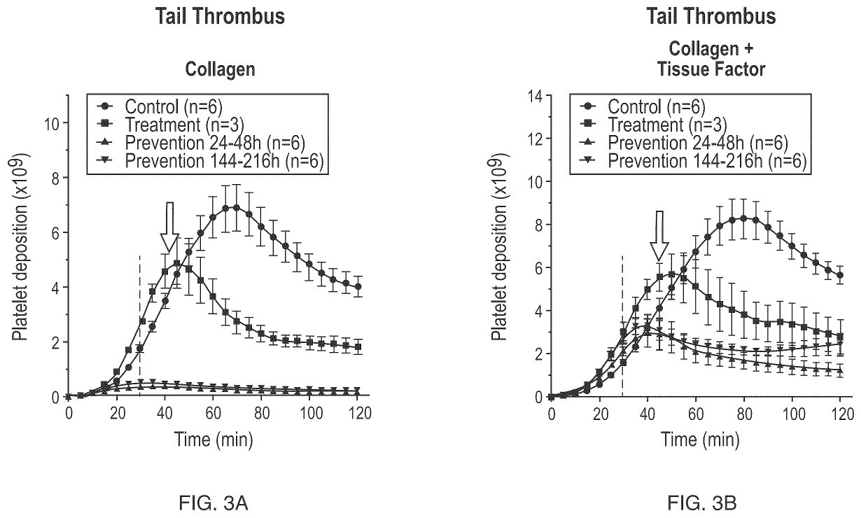

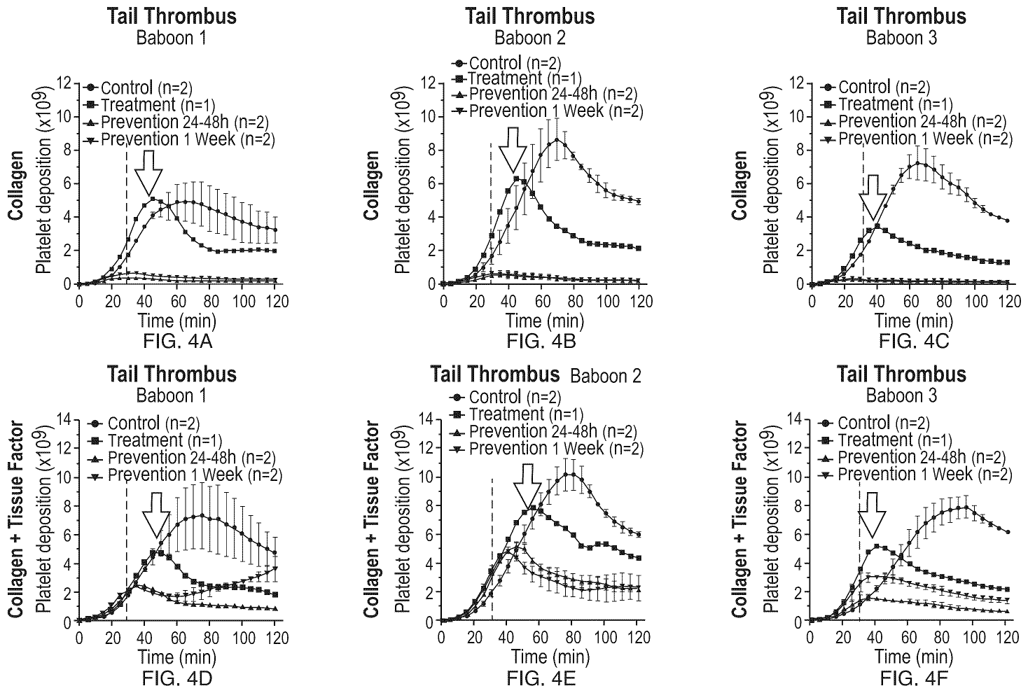

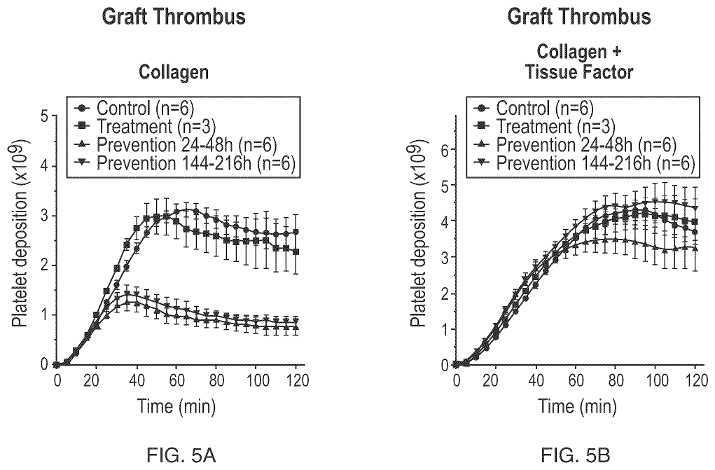

Administration of Antibody 1 stopped platelet deposition in the tail segment when administered 30 minutes after initiation of thrombosis. The Antibody 1 antithrombotic effect appeared to start within 20 minutes after administration, as shown in FIG. 3A for collagen coated and FIG. 3B for collagen+TF coated segments. Antibody 1 abolished platelet deposition when thrombosis was triggered after the start of Antibody 1 treatment. Platelet deposition for each of the three baboons for collagen-coated segments is shown in FIG. 4A, FIG. 4B, and FIG. 4C; platelet deposition for each of the three baboons for collagen+TF coated segments is shown in FIG. 4D, FIG. 4E, and FIG. 4F. The effect of Antibody 1 in stopping downstream propagation of platelet deposition was consistently observed in the three baboons. The effect of Antibody 1 on platelet deposition was small in the coated graft segment, especially after triggering thrombosis with collagen+TF (FIG. 5B) to mimic the trigger of a wound (collagen only in FIG. 5A). The lack of effect of Antibody 1 on platelet deposition in the collagen+TF coated segment is consistent with hemostasis sparing potential of Antibody 1.

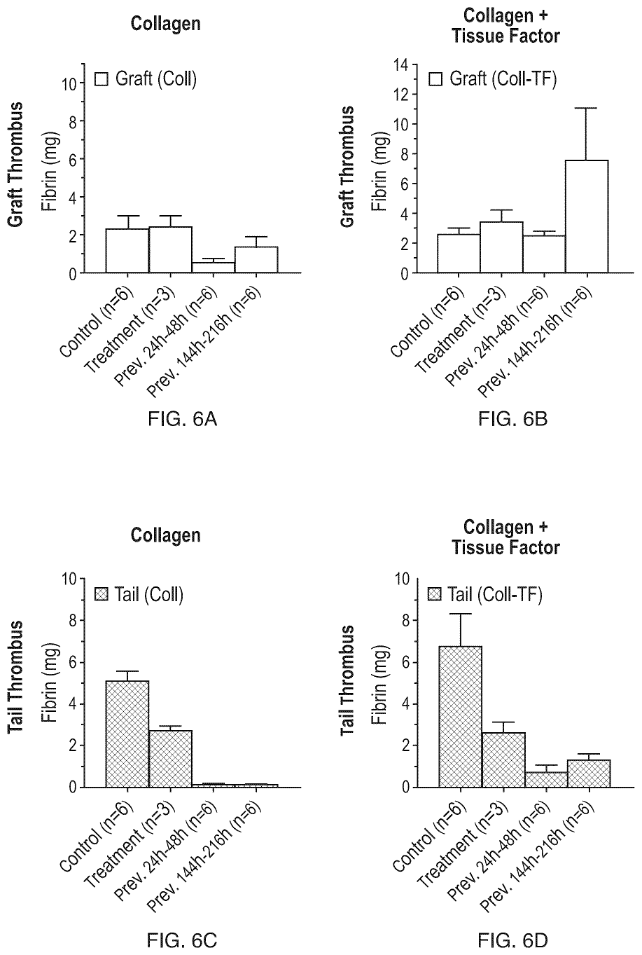

Antibody 1 also reduced fibrin deposition in the tail segment, indicating an antithrombotic efficacy when administered either before thrombosis or 30 minutes after thrombosis initiation (FIG. 6C, FIG. 6D). Fibrin deposition was not altered in the graft segment, again, indicating hemostasis sparing potential of Antibody 1 (FIG. 6A, FIG. 6B).

Intravenous Antibody 1 administration 30 minutes after thrombus induction stopped downstream thrombus growth and propagation of platelet deposition within 20 minutes after drug administration. Intravenous Antibody 1 was very effective in preventing downstream thrombus propagation when administered prior to initiation of thrombosis, consistent with clinical data. Data in the collagen+TF-coated grafts supported the concept of a hemostasis sparing effect of targeting FXI, thus, making Antibody 1 suitable for safe treatment and/or prevention of acute thrombosis.

In conclusion, the above studies demonstrate a potential for therapeutic benefit of targeting FXI/FXIa by Intravenous Antibody 1, both in therapeutic and in preventive settings.

### Example 4—Treatment of Patients with Vascular Graft Thrombosis

### Purpose and Rationale

This study aims to test whether Antibody 1 is effective in halting clot formation and downstream growth when administered before or during active clot formation in human subjects treated with an AV shut.

### Methods

Patients undergoing treatment with an AV shunt are treated with a single dose Antibody 1 at 150 mg intravenous (iv) or a control with an appropriate dosing regimen. Pharmacodynamic effect is measured by activated partial thromboplastin time (aPTT).

### Results

Consistent with its half-life of 20 to 30 days, single IV administration of Antibody 1 at a dose of 150 mg can result in long-lasting aPTT prolongation.

These data suggest that Antibody 1 has the potential to slow down the growth and reduce the size of thrombi when administered before clot induction. Such data also indicate a therapeutic benefit of targeting FXI both in therapeutic and preventative settings.

### Example 5—Treatment of Patients with Vascular Graft Thrombosis

### Purpose and Rationale

This study was designed to test the effects of increasing concentrations of Antibody 1 on platelet aggregation following stimulation with collagen or thrombin receptor activating peptide-6 (TRAP-6) compared to vehicle and active control (abciximab, anti-GP2b3a) and to determine the effects of both agents on thrombin generation.

### Methods

Whole blood was obtained in EDTA and citrate tubes (6 healthy donors). Specimens were spiked with vehicle, Antibody 1 (250, 500, or 1000 nM), or abciximab (50 nM). Platelet aggregation was recorded following induction using collagen (1 μg/mL) or TRAP-6 (8 μM) using a multiplate impedance aggregometer. The area under the curve (AUC) for platelet aggregation was determined in arbitrary aggregation*time (AU*min). Thrombin generation was also measured at the above Antibody 1 and abciximab concentrations in platelet-rich plasma using Thrombinoscope CAT (Calibrated Automated Thrombogram; Stago CH S.A. and tissue factor reagent (1 pM); fluorescence was measured by an automated plate reader fluorometer.

### Results

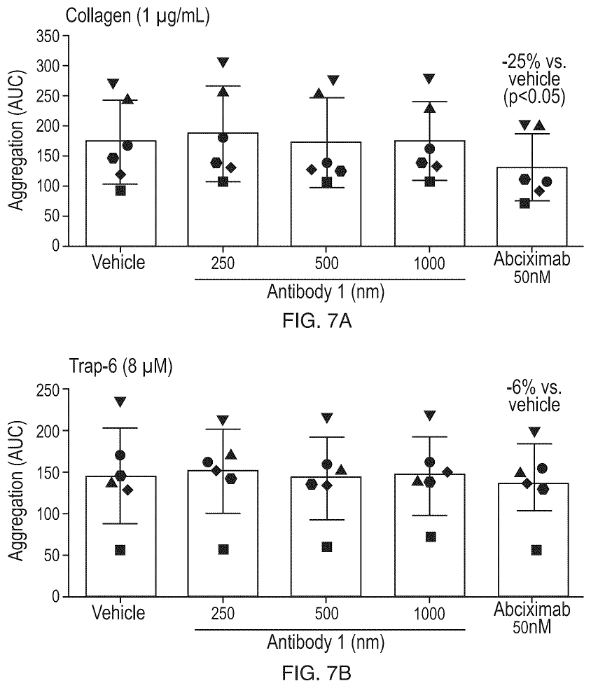

Antibody 1 showed no inhibitory or stimulatory effects on platelet aggregation induced by collagen (FIG. 7A) or TRAP-6 (FIG. 7B). In contrast, significant reductions in collagen-induced platelet aggregation were observed with abciximab. Antibody 1 resulted in significant delays in lag time and the time to peak concentration of thrombin generation; abciximab had no effect on thrombin generation.

Importantly, these results indicate that targeting FXI/FXIa by Antibody 1 does not affect platelet aggregation while inhibiting thrombin generation.

What is claimed is:

1. A method of treating a disease or disorder in a subject in need thereof, the method comprising intravenously administering to the subject a first dose of about 150 mg of an isolated anti-Factor XI (FXI) and/or anti-activated Factor XI (FXIa) antibody, or an antigen-binding fragment thereof, and subcutaneously administering to the subject a second dose of the isolated anti-FXI and/or anti-FXIa antibody, or the antigen-binding fragment thereof.

2. The method of claim 1, wherein the second dose comprises about 150 mg of the isolated anti-FXI and/or anti-FXIa antibody, or the antigen-binding fragment thereof.

3. The method of claim 1 or 2, wherein the first dose of the isolated anti-FXI and/or anti-FXIa antibody, or the antigen-binding fragment thereof, is formulated as an intravenous drug delivery formulation comprising about 150 mg of the antibody, or the antigen-binding fragment thereof.

4. The method of any one of claims 1-3, wherein the second dose of the isolated anti-FXI and/or anti-FXIa antibody, or the antigen-binding fragment thereof, is formulated as a subcutaneous drug delivery formulation comprising about 150 mg of the antibody or the antigen-binding fragment thereof.

5. The method of any one of claims 1-4, wherein the antibody is a human monoclonal antibody.

6. The method of claim 5, wherein the antibody is a human IgG1 isotype.

7. The method of claim 5 or 6, wherein the antibody comprises D265A and P329A substitutions in the Fc domain.

8. The method of any one of claims 1-7, wherein the antibody or antigen-binding fragment thereof is administered in a drug delivery formulation comprising a histidine buffer at a concentration of about 20 mM.

9. The method of any one of claims 1-8, wherein the antibody or antigen-binding fragment thereof is administered in a drug delivery formulation comprising sucrose at a concentration of about 220 mM.

10. The method of any one of claims 1-9, wherein the antibody or antigen-binding fragment thereof is administered in a drug delivery formulation comprising polysorbate 20 at a concentration of about 0.04%.

11. The method of any one of claims 1-10, wherein the antibody or antigen-binding fragment thereof is administered in a drug delivery formulation at pH 5.5.

12. The method of any one of claims 1-11, wherein, when the antibody or antigen-binding fragment thereof is administered in an intravenous drug delivery formulation, the intravenous drug delivery formulation further comprises about 5% glucose.

13. The method of any one of claims 1-12, wherein the subject has a cancer.

14. The method of any one of claims 1-13, wherein the subject has a cancer selected from the group consisting of gastrointestinal cancer and genitourinary cancer.

15. The method of any one of claims 1-14, wherein the subject is at high risk of venous thromboembolism.

16. The method of any one of claims 1-15, wherein the subject has had one or more previous venous thromboembolisms.

17. The method of any one claims 1-16, wherein the method further comprises one or more additional subcutaneous doses of the antibody or antigen-binding fragment thereof.

18. The method of any one of claims 1-17, wherein the method comprises administering five subcutaneous doses of the antibody or antigen-binding fragment thereof.

19. The method of any one of claims 1-18, wherein the antibody or antigen-binding fragment thereof is administered subcutaneously about once a month.

20. The method of any one of claims 1-19, wherein the antibody or antigen-binding fragment thereof is administered intravenously on day 1 and is administered subcutaneously on days 31, 61, 91, 121, and 151.

21. The method of any one of claims 1-20, wherein the subject is treated for about six months.

22. A method of treating a subject with a cancer, wherein the method comprises administering a drug delivery formulation comprising about 150 mg of an isolated anti-Factor XI (FXI) and/or anti-activated Factor XI (FXIa) antibody or antigen-binding fragment thereof to the subject in need thereof, wherein the drug delivery formulation is administered once intravenously and subsequently is administered subcutaneously about once a month, and wherein the subject is treated for about six months.

23. The method of claim 22, wherein the cancer is selected from the group consisting of gastrointestinal cancer and genitourinary cancer.

24. A method of treating a primate subject at risk of thrombosis, wherein the method comprises administering to the primate subject a single dose of a drug delivery formulation comprising:
(a) a therapeutically effective amount of an isolated anti-Factor XI (FXI) and/or anti-activated Factor XI (FXIa) antibody, or antigen-binding fragment thereof at a concentration of about 150 mg;
(b) a histidine buffer at a concentration of about 20 mM;
(c) sucrose at a concentration of about 220 mM; and
(d) polysorbate-20 at a concentration of about 0.04% (v/v),
(e) a diluent comprising glucose,
at pH 5.5,
and wherein the administering is before or during formation of a blood clot.

25. The method of claim 24, wherein the primate subject is a baboon.

26. The method of claim 24, wherein the primate subject is a human.

27. The method of claim 24 or 25, wherein the thrombosis is an experimentally-induced thrombosis.

28. The method of any one of claims 24-27, wherein the primate subject is at risk of vascular graft thrombosis.

29. The method of any one of claims 24-28, wherein the single dose is administered to prevent thrombosis.

30. The method of any one of claims 24-28, wherein the single dose is administered to treat thrombosis.

31. The method of any one of claims 24-30, wherein the single dose is parenteral or intravenous.

32. The method of any one of claims 24-31, wherein the single dose is parenteral.

33. The method of any one of claim 24-25 or 27-32, wherein about 1 mg/kg is the therapeutically effective amount of the anti-Factor XI (FXI) and/or anti-activated Factor XI (FXIa) antibody or antigen-binding fragment thereof, for administration to the primate subject.

34. The method of any one of claim 24, 26, or 28-32, wherein about 150 mg is the therapeutically effective amount of the anti-Factor XI (FXI) and/or anti-activated Factor XI (FXIa) antibody or antigen-binding fragment thereof, for administration to the primate subject.

35. A method of treating a subject having a thrombocytopenia, wherein the thrombocytopenia is selected from the group consisting of: chemotherapy-induced thrombocytopenia, congenital thrombocytopenia, thrombocytopenia associated with infection, and idiopathic thrombocytopenia, the method comprising administering a therapeutically effective amount of a Factor XI and/or Factor XIa antibody, or an antigen-binding fragment thereof, to the subject in need thereof.

36. The method of claim 35, wherein the subject having a thrombocytopenia has a cancer.

37. The method of claim 35 or 36, wherein the subject having a thrombocytopenia has cirrhosis.

38. The method of claim 35 or 36, wherein the subject having a thrombocytopenia has idiopathic thrombocytopenia purpura (ITP).

39. A method of treating a cancer subject having a chemotherapy-induced thrombocytopenia, wherein the method comprises administering a therapeutically effective amount of a Factor XI and/or Factor XIa antibody, or an antigen-binding fragment thereof, to the cancer subject in need thereof.

40. The method of any one of claims 35-39, wherein the subject or the cancer subject is afflicted with or at risk of developing a thromboembolic disorder.

41. The method of any one of claims 1-40, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

42. The method of any one of claims 1-41, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of KNY; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of KNY; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

43. The method of any one of claims 1-42, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

44. The method of any one of claims 1-43, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

45. The method of any one of claims 1-44, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

46. The method of any one of claims 1-45, wherein the antibody comprises a heavy chain comprising an amino acid sequence of SEQ ID NO: 31 and a light chain comprising an amino acid sequence of SEQ ID NO: 41.

47. The method of any one of claims 1-46, wherein the antibody is a human monoclonal antibody.

48. The method of claim 47, wherein the antibody is a human IgG1 isotype.

49. The method of claim 47 or 48, wherein the antibody comprises D265A and P329A substitutions in the Fc domain.

50. The method of any one of claims 1-49, wherein the administering of the antibody or antigen-binding fragment thereof does not affect platelet aggregation in the subject as compared to platelet aggregation prior to the administering.

51. The method of claim 50, wherein the platelet aggregation is measured by impedance platelet aggregometry.

52. The method of claim 51, wherein the platelet aggregation is induced by collagen, adenosine 5′-diphosphate (ADP), or thrombin receptor activating peptide-6 (TRAP-6).

53. The method of any one of claims 50-52, wherein the platelet aggregation is determined ex vivo or in vitro.

54. The method of any one of claims 35-53, wherein the antibody or antigen-binding fragment thereof is administered intravenously.

55. The method of any one of claims 35-54, wherein the antibody or antigen-binding fragment thereof is administered subcutaneously.

56. The method of any one of claims 35-55, wherein a first dose of the antibody or antigen-binding fragment thereof is administered intravenously and a second dose of the antibody or antigen-binding fragment is administered subcutaneously.

57. The method of claim 56, further comprising one or more additional doses of the antibody or antigen-binding fragment thereof administered subcutaneously following the administering of the second dose.

58. The method of any one of claims 35-57, wherein the antibody or antigen-binding fragment thereof is administered once a month.
